(19) United States
(12) Patent Application Publication
(10) Pub. No.: US 2023/0310623 A1
(43) Pub. Date: Oct. 5, 2023

(54) COMPOSITIONS AND METHODS FOR TARGETING TUMOR ASSOCIATED TRANSCRIPTION FACTORS

(71) Applicant: Beth Israel Deaconess Medical Center, Inc., Boston, MA (US)

(72) Inventors: Bon Q. TRINH, Chestnut Hill, MA (US); Daniel Geoffrey TENEN, Boston, MA (US)

(21) Appl. No.: 18/014,561

(22) PCT Filed: Jul. 16, 2021
(86) PCT No.: PCT/US2021/041934

§ 371 Date: Jan. 5, 2023

Related U.S. Application Data
(60) Provisional application No. 63/054,531, filed on Jul. 21, 2020.

(51) Int. Cl. A61K 47/64; C07K 19/00; C07K 14/575; A61K 47/54; A61K 38/46; A61K 31/7088; A61K 48/00; C12N 15/86; C12N 15/11; C12N 9/22; C12N 15/90; A61P 35/02; A61P 35/00

(52) U.S. Cl. CPC A61K 47/64; C07K 19/00; C07K 14/575; A61K 47/549; A61K 38/465; A61K 31/7088; A61K 48/0041; C12N 15/86; C12N 15/11; C12N 9/22; C12N 15/907; A61P 35/02; A61P 35/00; C12N 2310/20; C12N 2800/80; C12N 2740/15043; C12N 2740/15052; C12N 2740/15071

(57) ABSTRACT

Described are compositions and methods for targeting tumor associated transcription factors (e.g., PU.1) using IncRNA, constructs comprising IncRNA, and CRISPR/Cas systems, and polynucleotides encoding IncRNA, constructs comprising IncRNA, and CRISPR/Cas systems, vectors containing the polynucleotides, viral or non-viral delivery vehicles containing the vectors, and compositions (e.g., pharmaceutical compositions) containing the same for use in methods treatment.

### STATEMENT REGARDING FEDERALLY SPONSORED RESEARCH

This invention was made with government support under grant CA222707 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on 12 July, 2021, is named 01948-279WO2_Sequence_Listing_7_12_21_ST25 and is 39,024 bytes in size.

### BACKGROUND

Long-range enhancer-promoter interactions result in dynamic expression patterns of lineage genes. How these communications occur in specific cell types and at specific gene loci remain elusive. Here we investigate whether RNAs coordinate with transcription factors to drive lineage gene transcription. In an integrated genome-wide approach surveying for gene loci exhibiting concurrent RNA- and DNA-interactions with RUNX1 protein, we identified a long noncoding RNA (lncRNA) arising from the upstream region of the myeloid master regulator PU.1. This myeloid-specific and polyadenylated lncRNA acts as a transcriptional inducer of PU.1 by modulating the formation of an active chromatin loop at the PU.1 locus. The lncRNA utilizes embedded transposable element variants to bind and recruit RUNX1 to both the enhancer and the promoter, resulting in the formation of the enhancer-promoter complex. These findings provide mechanistic insight, highlighting the important role of the interplay between cell type-specific RNAs and transcription factors in lineage-gene activation.

Lineage-control genes that dictate cellular identities are often expressed in dynamic and hierarchical patterns. Disturbance of these established normal patterns associates with anomalies (Iwasaki et al., Genes Dev. 20: 3010-3021, 2006; Novershtern et al., Cell 144: 296-309, 2011; Shivdasani and Orkin, Blood 87: 4025-4039, 1996; Tenen et al., Blood 90: 489-519, 1997). Understanding cell type-specific gene regulation, therefore, will provide important mechanistic insights into development and disease. Multiple key players including transcription factors and growth factor signaling pathways are implicated to act in concert in driving gene expression (Palani and Sarkar, PLoS Comput. Biol. 5: el 000518, 2009; Sarrazin and Sieweke, Semin. Immunol. 23: 326-334, 2011). In the blood system, the ETS-family transcription factor PU.1 (also known as Spi-1) induces expression of receptors for important growth factors such as M-CSF, GM-CSF and G-CSF which instruct myeloid differentiation (Hohaus et al., 1995; Iwasaki et al., Blood 106: 1590-1600, 2005; Smith et al., Blood 88: 1234-1247, 1996; Zhang et al., Mol. Cell Biol. 14: 373-381, 1994). PU.1 is silent in most tissues and cell types but elevated in the myeloid cells including granulocytes and monocytes. Downregulation of PU.1 impairs myeloid cell differentiation leading to acute myeloid leukemia (AML) (Cook et al., Blood 104:3437-3444, 2004; Rosenbauer et al., Nat. Genet. 36: 624-630, 2004; Tenen, Nat. Rev. Cancer 3: 89-101, 2003; Walter et al., PNAS 102: 12513-12518, 2005). Runt-related transcription factor 1 (RUNX1) is known as a critical upstream regulator of PU.1 in myeloid development (Huang et al., Nat. Genet. 40: 51-60, 2008; Okada et al., Oncogene 17: 2287-2293, 1998). Yet, RUNX1 is expressed in many different cell types and plays diverse biological roles not only in hematopoiesis but also in development of neurons, hair follicles, and skin (Chen et al., Neuron 49: 365-377, 2006; Hoi et al., Mol. Cell Biol. 30: 2518-2536, 2010; North et al., Immunity 16: 661-672 2002; Osorio et al., J. Cell Biol. 193: 235-250, 2011). In general, transcription factors that regulate cell type-specific genes are also ubiquitously expressed and exert their regulatory roles in diverse cell types (O'Connor et al., Yale J. Biol. Med. 89: 513-525, 2016). Thus, how cell type- and gene-specific induction takes place still remains a paradox. This leads us to postulate that unknown ad hoc regulators act in orchestration with transcription factors to drive cell type-specific gene transcription.

Transcription of many cell type-specific genes are induced by enhancer elements, which are located at variable distances from gene targets (Bulger and Groudine, Cell 144: 327-339, 2011; Levine, Curr. Biol. 20: R754-R763, 2010). For instance, PU.1 transcription is induced by the formation of a specific chromatin loop resulting from the interaction between the upstream regulatory element (URE) (−17 kb in human and −14 kb in mouse) and the proximal promoter region (PrPr) (Ebralidze et al., Genes Dev. 22: 3096-2092, 2008; Li et al., Blood 98: 2958-2965, 2001; Staber et al., Mol. Cell 49: 934-946, 2013). Interestingly, abrogation of RUNX1-binding motifs at the URE reduces URE-PrPr interaction causing decreased PU.1 expression in myeloid cells (Huang et al., 2008, supra; Staber et al., Blood 124: 2391-2399, 2014). Because RUNX1 is ubiquitously expressed, it remains unclear how this transcription factor modulates chromatin structure in such gene- and cell type-specific manners. Notably, several lines of evidence also suggest that transcription factors, such as Tumor protein p53 (p53), Signal Transducer and Activator of Transcription 1 (STAT1), and CCCTC-binding factor (CTCF) are capable of binding to RNAs (Cassiday and Maher, Nucleic Acids Res. 30:4118-4126, 2002; Kung et al., Mol. Cell 57: 361-375, 2015; Miller et al., Mol. Cell Biol. 20: 8420-8431, 2000; Mosner et al., EMBO J 14: 4442-4449, 1995; Peyman, Biol. Reprod. 60: 23-31, 1999; Saldana-Meyer et al., Genes Dev. 28: 723-734, 2014). Thus, it is tempting to hypothesize that RUNX1 coordinates with RNAs, which exist specifically in myeloid cells, to drive long-range transcription of PU.1.

With advances in whole transcriptome sequencing in the last decade, thousands of noncoding RNAs (ncRNA) has been unveiled (Djebali et al., Nature 489: 101-108, 2012). Arbitrarily defined as ncRNAs having at least 200 nucleotides in length, long noncoding RNAs (lncRNA) are implicated to display tissue-specific expression patterns (Ponting et al., Cell 136: 629-641, 2009; Uszczynska-Ratajczak et al., Nat. Rev. Genet. 19: 535-548, 2018) and might undergo post-transcriptional processing such as splicing and polyadenylation (Mercer et al., Nat. Rev. Genet. 10: 155-159, 2009). Through interactions with DNAs, proteins, and other RNAs, lncRNAs regulate fundamental cellular processes such as transcription, RNA stability, and DNA methylation (Di Ruscio et al., Nature 503: 371-376, 2013; Mercer et al., 2009, supra; Rinn and Chang, Annu. Rev. Biochem 81: 145-166, 2012). Of note, transcription also occurs at active enhancers, giving rise to enhancer RNAs (eRNA) which include 1d-eRNAs (long, polyadenylated and unidirectional transcription) and 2d-eRNAs (short, non-polyadenylated and bidirectional transcription) (Li et al., Nat. Rev. Genet. 17: 207-223, 2016; Natoli and Andrau, Annu. Rev. Genet. 46: 1-19, 2012). Mounting evidence suggests that 2d-eRNAs are involved in transcriptional enhancement by strengthening enhancer-promoter loop (Lam et al., Nature 498: 511-515, 2013; Li et al., Nature 498: 516-520, 2013; Melo et al., Mol Cell 49: 524-535, 2013). However, it is not clear whether and how these eRNAs control enhancer-promoter interaction in a gene-specific manner. To date, only a few lncRNAs have been precisely mapped and functionally defined (Uszczynska-Ratajczak et al., 2018, supra), leaving most lncRNAs poorly annotated and largely unexplored.

Acute myloid leukemia (AML) is characterized by impaired differentiation and uncontrolled proliferation with subsequent accumulation of immature cells (blasts). Although treatment results in AML have improved over the past 30 years, more than 50% of young adults and 90% of older patients succumb to their disease. Differentiation therapy with all-trans retinoic acid (ATRA) can have markedly improved outcome in certain types of acute myeloid leukemia (AML) (e.g., acute promyelocytic leukemia (APL)) while having little clinical impact on other AML sub-types. Advances in diagnosing a subject as having a cancer that would be sensitive or resistant to ATRA treatment are needed.

### SUMMARY OF THE DISCLOSURE

One aspect of the disclosure features a polynucleotide including a sequence with at least 20 nucleotides (e.g., at least about 25, at least about 40, at least about 60, at least about 80, at least about 100, at least about 150, at least about 300, at least about 500, at least about 900, at least about 1300, at least about 1700, at least about 2000, at least about 2300, at least about 2350, or at least about 2375) of SEQ ID NO: 1, and variants thereof with at least 85% (e.g., 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto, wherein the polynucleotide has fewer than 2,381 (e.g., 2380, 2000, 1900, 1600, 1500, 1400, 1300, 1200, 1100, 1000, 900, 800, 700, 600, 500, 400, 300, 250, 225, 200, 175, 150, 125, 100, 75, 50, 40, 30, or 20) nucleotides of SEQ ID NO: 1. In some embodiments, the polynucleotide may include a nucleic acid sequence with between about 20 nucleotides and about 2380 nucleotides (e.g., between about 20 and about 100, between about 70 and about 300, between about 200 and about 500, between about 400 and about 800, between about 700 and about 1200, between about 1100 and about 1600, between about 1500 and about 2000, or between about 1900 and about 2380) or SEQ ID NO: 1, or variants thereof with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto.

In some embodiments, the polynucleotide includes a binding region for a Runt-related transcription factor 1 (RUNX1) protein or fragment thereof. In some embodiments, the binding region includes all or at least 20 nucleotides (e.g., at least 25, at least 40, at least 60, at least 80, at least 100, at least 150, at least 300, at least 500, at least 900, at least 1300, at least 1700, at least 2000, at least 2300, at least 2350, or at least 2375 nucleotides) of one or more transposable elements (TEs). In some embodiments, the one or more TEs includes a nucleotide sequence with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to at least 20 or more nucleotides (e.g., e.g., at least 25, at least 40, at least 60, at least 80, at least 100, at least 150, at least 300, at least 500, at least 900, at least 1300, at least 1700, at least 2000, at least 2300, at least 2350, or at least 2375 nucleotides or more nucleotides) of any one of SEQ ID NOs: 2-4. In some embodiments, the polynucleotide includes two said TEs or three said TEs. In some embodiments, the polynucleotide includes three said TEs, and wherein a first said TE includes at least 20 nucleotides (e.g., at least 25, at least 40, at least 60, at least 80, at least 100, at least 150, at least 300, at least 500, at least 900, at least 1300, at least 1700, at least 2000, at least 2300, at least 2350, or at least 2375 nucleotides) of SEQ ID NO: 2, a second said TE includes at least 20 nucleotides (e.g., at least 25, at least 40, at least 60, at least 80, at least 100, at least 150, at least 300, at least 500, at least 900, at least 1300, at least 1700, at least 2000, at least 2300, at least 2350, or at least 2375 nucleotides) of SEQ ID NO: 3, and a third said TE includes at least 20 nucleotides (e.g., at least 25, at least 40, at least 60, at least 80, at least 100, at least 150, at least 300, at least 500, at least 900, at least 1300, at least 1700, at least 2000, at least 2300, at least 2350, or at least 2375 nucleotides) of SEQ ID NO: 4.

In some embodiments, the three said TEs include SEQ ID NOs: 2-4. In some embodiments, the first, second, and third TEs are present in the polynucleotide in order, 5′ to 3′, and where the TEs are linked directly or through a linker.

In some embodiments, the polynucleotide includes at least 30 nucleotides (e.g., at least 40, at least 100, at least 500, at least 1700, at least 2000, at least 2300, or at least 2375 nucleotides) of SEQ ID NO: 1.

In another aspect, the disclosure features a construct including a RUNX1 protein, or fragment thereof, conjugated to at least one polynucleotide of any one of claims 1-18. In some embodiments, the construct includes at least one said RUNX1 protein, or fragment thereof, bound to at least one said polynucleotide. In some embodiments, the RUNX1 protein, or fragment thereof, and the polynucleotide are bound through a covalent bond.

In some embodiments, the construct includes the structure:

R-L-P (I) or P-L-R (II),

wherein R is the RUNX1 protein or fragment thereof;

P is the polynucleotide; and

L is a linker.

In some embodiments, the construct includes the structure of R-L-P (I). In certain embodiments, the construct includes the structure of P-L-R (II). In other embodiments, R includes at least 100 amino acids (e.g., at least 150, at least 175, at least 200, at least 225, at least 250, at least 275, at least 300, at least 325, at least 350, at least 375, at least 400, at least 425, at least 450, or at least 475 amino acids) of SEQ ID NO: 5, and variants thereof with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. In some embodiments, R polypeptide has the sequence of SEQ ID NO: 5.

In some embodiments, the R component of the construct is a RUNX polypeptide that includes at least one binding site for at least one polynucleotide regulatory element of PU.1. In certain embodiments, the at least one PU.1 regulatory element has at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to the sequence of SEQ ID NO: 6.

In some embodiments, the at least one PU.1 regulatory element has the sequence of SEQ ID NO: 6. In some embodiments, the at least one PU.1 regulatory element is an upstream regulatory element (URE) and/or a proximal promoter region (PrPr). In certain embodiments, the PrPr has at least 85% sequence identity (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) to the sequence of SEQ ID NO: 7. In some embodiments, the PrPr has the sequence of SEQ ID NO: 7.

In another aspect, the disclosure features a polynucleotide encoding the construct of any one of above embodiments described herein.

In another aspect, the disclosure features a vector including the polynucleotide of any of the above embodiments described herein. In some embodiments, the vector is an expression vector or a viral vector (e.g., a lentiviral vector).

In another aspect, the disclosure features a cell (e.g., a mammalian cell, such as a human cell) containing the polynucleotide or the vector of any of the above embodiments described herein.

In another aspect, the disclosure features a composition including the polynucleotide of any one of the above embodiments, the construct of any one of the above embodiments, the vector of the above embodiments, or the cell of the above embodiments. In some embodiments, the composition further includes a pharmaceutically acceptable carrier, excipient, or diluent.

In another aspect, the disclosure features a method of treating a medical condition in a subject in need thereof by administering polynucleotide, construct, vector, and/or cell of any one of the above embodiments.

In some embodiments, the medical condition is a cancer (e.g., a blood cancer (e.g., acute myeloid leukemia (AML) or myeloma), or a liver cancer (e.g., metastatic hepatocellular carcinoma (HCC))).

In another aspect, the disclosure features a method of treating a medical condition in a subject in need thereof including administering the construct of any one of the embodiments described herein. In several embodiments, the medical condition is a cancer (e.g., a blood cancer (e.g., acute myeloid leukemia (AML) or myeloma), or a liver cancer (e.g., metastatic hepatocellular carcinoma (HCC))).

In another aspect, the disclosure features the use of the construct of any one of the embodiments described herein in the preparation of a medicament for the treatment of a medical condition in a subject in need thereof.

In another aspect, the disclosure features a method of treating a medical condition in a subject, in which the method includes:
a) delivering to a target cell a dCas activator system including:
   i) a plurality of first guide ribonucleic acids (gRNAs) directed to a first genomic site of an endogenous DNA molecule of the cell; and
   ii) a plurality of dCas fusion proteins;
in which the first gRNA forms a first complex with a first said dCas fusion protein at the first genomic site, and in which the first complex promotes the expression of LOUP. In some embodiments, the first guide gRNA specifically hybridizes to the first genomic site. In some embodiments, the first genomic site and the target gene of interest are between 10-100,000 nucleotide base pairs apart (e.g., between 50-150, between 100-800 (e.g., between 125-200, between 175-300, between 275-400, between 375-500, between 475-600, between 575-700, and between 675-800), between 700-2000, between 1000-5000, between 4000-10000, between 9000-20000, between 19000-30000, between 25000-50000, between 45000-75000, or between 70000-100000). In some embodiments, the first genomic site includes a protospacer adjacent motif (PAM) recognition sequence positioned upstream from the first genomic site. In some embodiments, the first guide RNA is a single guide RNA (sgRNA). In some embodiments, the dCas fusion protein is selected from a group including dCas9-VP64, dCas9-VPR, dCas9-SAM, dCas9-Scaffold, dCas9-Suntag, dCas9-P300, dCas9-VP160, and VP64-dCas9-BFP-VP64. In some embodiments, the dCas fusion protein is dCas9-VP64. In certain embodiments, the first target genomic site is associated with the medical condition. In some embodiments, the medical condition is a cancer. In another embodiment, the cancer is a cancer associated with tumor suppressor gene PU.1. In some embodiments, the cancer associated with tumor suppressor gene PU.1 is acute myeloid leukemia (AML), liver cancer, or myeloma. In certain embodiments, the target gene of interest is tumor suppressor gene PU.1.

In another aspect, the disclosure features a nucleic acid including a polynucleotide including a nucleic acid sequence encoding a dCas activator system. In certain embodiments, the dCas activator system includes a dCas fusion protein. In some embodiments, the nucleic acid further includes a nucleic acid sequence encoding a first gRNA. In some embodiments, the first gRNA is directed to a first genomic site of an endogenous DNA molecule of a cell. In certain embodiments, the nucleic acid molecule further includes a promoter. In certain embodiments, the dCas fusion protein is selected from a group including dCas9-VP64, dCas9-VPR, dCas9-SAM, dCas9-Scaffold, dCas9-Suntag, dCas9-P300, dCas9-VP160, and VP64-dCas9-BFP-VP64.

In another aspect, the disclosure features a vector including the nucleic acid of the previous aspect and embodiments thereof. In some embodiments, the vector is an expression vector or a viral vector (e.g., a lentiviral vector).

In another aspect, the disclosure features a composition including:
a) a plurality of first guide ribonucleic acids (gRNAs) directed to a first genomic site of an endogenous DNA molecule of the cell; and
b) a plurality of dCas fusion proteins. In some embodiments, the first gRNA is in a first complex with a first said dCas fusion protein,
in which the first complex is configured to promote the expression of a target gene of interest. In some embodiments, the dCas fusion protein is selected from the group including dCas9-VP64, dCas9-VPR, dCas9-SAM, dCas9-Scaffold, dCas9-Suntag, dCas9-P300, dCas9-VP160, and VP64-dCas9-BFP-VP64. In a particular embodiment, the dCas fusion protein is dCas9-VP64.

In another aspect, the disclosure features a pharmaceutical composition including the nucleic acid of any one of the above aspects and/or embodiments, or the composition of any one of the above aspects and embodiments, and a pharmaceutically acceptable carrier, excipient, or diluent.

In another aspect, the disclosure features a kit including the nucleic acid of any one of the above referenced aspects and/or embodiments, the composition of any one of the above referenced aspects and/or embodiments, or the pharmaceutical composition of the above aspect, and a package insert including instructions for using the nucleic acid, composition, or pharmaceutical composition for treating a medical condition in a subject.

In another aspect, the disclosure features a method of treating a medical condition in a subject, wherein the method includes:
a) delivering to a target cell a gene editing system including:
   i) a plurality of first guide ribonucleic acids (gRNAs) directed to a first genomic site of an endogenous DNA molecule of the cell; and
   ii) a plurality of RNA programmable nucleases;
wherein the first guide RNA forms a first complex with a first said RNA programmable nuclease at the first genomic site, and wherein the first complex promotes the inhibition of expression of LOUP. In some embodiments, the first guide gRNA specifically hybridizes to the first genomic site. In some embodiments, the first genomic site and the target gene of interest are between 10-100,000 nucleotide base pairs apart (e.g., between 50-150, between 100-800 (e.g., between 125-200, between 175-300, between 275-400, between 375-500, between 475-600, between 575-700, and between 675-800), between 700-2000, between 1000-5000, between 4000-10000, between 9000-20000, between 19000-30000, between 25000-50000, between 45000-75000, or between 70000-100000). In some embodiments, the first genomic site includes a protospacer adjacent motif (PAM) recognition sequence positioned upstream from said first genomic site. In certain embodiments, the first guide RNA is a single guide RNA (sgRNA). In another embodiment, the inhibition of expression of the target gene of interest is caused by non-homologous end-joining (NHEJ). In other embodiments, the first target genomic site is associated with the medical condition. In another embodiment, the medical condition is associated with tumor suppressor gene PU.1. In certain embodiments, the medical condition associated with PU.1 is Alzheimer's Disease or asthma. In another embodiment, the target gene of interest is tumor suppressor gene PU.1. In certain embodiments, the RNA program nuclease is a Cas RNA programmable nuclease. In some embodiments, the Cas RNA programmable nuclease is a Cas9 RNA programmable nuclease.

In another aspect, the disclosure features a nucleic acid including a polynucleotide including a nucleic acid sequence encoding:
a) a first gRNA directed to a first genomic site of an endogenous DNA molecule of a target cell; and
b) an RNA-programmable nuclease;
in which the first genomic site is between 10-100,000 nucleotide base pairs (e.g., between 50-150, between 100-800 (e.g., between 125-200, between 175-300, between 275-400, between 375-500, between 475-600, between 575-700, and between 675-800), between 700-2000, between 1000-5000, between 4000-10000, between 9000-20000, between 19000-30000, between 25000-50000, between 45000-75000, or between 70000-100000) from a target gene of interest including tumor suppressor gene PU.1. In some embodiments, the nucleic acid further includes a promoter. In another embodiment, the RNA programmable nuclease is a Cas RNA programmable nuclease. In some embodiments, the Cas RNA programmable nuclease is a Cas9 RNA programmable nuclease.

In another aspect, the disclosure features a vector including a nucleic acid of the previous aspect or any embodiments thereof. In some embodiments, the vector is an expression vector or a viral vector. In another embodiment, the viral vector is a lentiviral vector.

Another aspect of the disclosure features a cell (e.g., a mammalian cells, such as a human cell) containing a polynucleotide or a vector as described above.

In another aspect, the disclosure features the use of RNAs (e.g., lncRNA (e.g., LOUP lncRNA)) to link transcription factors to genes. In some embodiments, linking transcription factors to genes modulates expression of the gene.

### Definitions

The term “about” means ±10% of the stated amount.

As used herein, the term “binds to” or “specifically binds to” refers to measurable and reproducible interactions such as binding between a guide polynucleotide and an RNA programmable nuclease, which is determinative of the presence of the target in the presence of a heterogeneous population of molecules including biological molecules. For example, an RNA programmable nuclease that binds to or specifically binds to a guide polynucleotide (which can be an engineered guide polynucleotide) is an RNA programmable nuclease that binds this guide polynucleotide with greater affinity, avidity, more readily, and/or with greater duration than it binds to other guide polynucleotides. In certain examples, an RNA programmable nuclease that specifically binds to a guide polynucleotide has a dissociation constant (Kd) of ≤1 μM, ≤100 nM, ≤10 nM, ≤1 nM, or ≤0.1 nM. In certain examples, an RNA programmable nuclease binds to a guide polynucleotide (e.g., guide RNA), wherein the RNA programmable nuclease and the guide polynucleotide form a complex at a target site (e.g., a target genomic site) on a target nucleic acid (e.g., a target genome). In another aspect, specific binding can include, but does not require exclusive binding.

The term “Cas” or “Cas nuclease” refers to an RNA-guided nuclease comprising a Cas protein (e.g., a Cas9 protein), or a fragment thereof (e.g., a protein comprising an active cleavage domain of Cas). A Cas nuclease is also referred to alternatively as an RNA-programmable nuclease, and a CRISPR/Cas system. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids.

CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas protein (e.g., a Cas9 protein). The tracrRNA serves as a guide for ribonuclease 3-aided processing of pre-crRNA. Subsequently, Cas/crRNA/tracrRNA cleaves linear or circular dsDNA target complementary to the spacer. The target strand not complementary to crRNA is first cut by endonuclease activity, then trimmed 3′-5′ by exonuclease activity. In nature, DNA-binding and cleavage typically requires Cas protein, crRNA, and tracrRNA. However, single guide RNAs (“sgRNA”, or simply “gRNA”) can be engineered so as to incorporate aspects of both the crRNA and tracrRNA into a single RNA species. See, e.g., Jinek et al. (Science 337:816-821, 2012), the entire contents of which is hereby incorporated by reference. RNA programmable nucleases (e.g., Cas9) recognize a short motif in the CRISPR repeat sequences (the protospacer adjacent motif (PAM)) to help distinguish self versus non-self. Cas9 nuclease sequences and structures are well known to those of skill in the art (see, e.g., Ferretti et al. (Proc. Natl. Acad. Sci. U.S.A. 98:4658-4663, 2001); Deltcheva et al. (Nature 471:602-607, 2011); and Jinek et al. (2012, supra), the entire contents of each of which are incorporated herein by reference). Cas9 orthologs have been described in various species, including, but not limited to, S. pyogenes and S. thermophilus. In some instances, it is desirable to use an inactive Cas or “dCas” RNA programmable nuclease. dCas nucleases are mutant forms of Cas nucleases whose endonuclease activity has been removed through point mutations in the endonuclease domains. Mutations in at least one of the two endonuclease domains, RuvC and HNH domains, in particular D10A and H840A change two important residues for endonuclease activity resulting in Cas9 deactivation. Additional suitable RNA programmable nucleases and sequences will be apparent to those of skill in the art based on this disclosure, and such RNA programmable nucleases and sequences include Cas9 sequences from the organisms and loci disclosed in, e.g., Chylinski et al. (RNA Biology 10:5, 726-737, 2013); the entire contents of which are incorporated herein by reference.

As used herein, a “coding region” is a portion of a nucleic acid that contains codons that can be translated into amino acids. Although a “stop codon” (TAG, TGA, TAA) is not translated into an amino acid, it may be considered to be part of a coding region, if present, but any flanking sequences, for example, promoters, ribosome binding sites, transcriptional terminators, introns, 5′ and 3′ untranslated regions, and the like, are not part of the coding region.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, the entire contents of each of which is incorporated herein by reference. The sequence surrounding the translational start site can be converted to a consensus Kozak sequence according to known methods. See, e.g., Kozak et al. (Nucleic Acids Res. 15 (20): 8125-8148, 1987), the entire contents of which is hereby incorporated by reference. Multiple stop codons can be incorporated.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine), and Hoogsteen base pairs.

The term “contiguous,” as used herein in the context of an oligonucleotide, refers to nucleosides, nucleobases, sugar moieties, or inter-nucleoside linkages that are immediately adjacent to each other. For example, “contiguous nucleobases” means nucleobases that are immediately adjacent to each other in a sequence.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is, therefore, interpreted to be an open term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

The terms “conjugating,” “conjugated,” and “conjugation” refer to an association of two entities, for example, of two molecules such as a protein and another molecule (e.g., a nucleic acid). In some aspects, the association is between a protein (e.g., RNA-programmable nuclease) and a nucleic acid (e.g., a guide RNA). In some instances, the association is between a protein (e.g., a RUNX1 protein or fragment thereof) and a nucleic acid (e.g., a LOUP polynucleotide). The association can be, for example, via a direct or indirect (e.g., via a linker) covalent linkage. In some embodiments, the association is covalent. In some embodiments, two molecules are conjugated via a linker connecting both molecules.

The term “consensus sequence,” as used herein in the context of nucleic acid sequences, refers to a calculated sequence representing the most frequent nucleotide residues found at each position in a plurality of similar sequences. Typically, a consensus sequence is determined by sequence alignment in which similar sequences are compared to each other and similar sequence motifs are calculated. In the context of nuclease target genomic site sequences, a consensus sequence of a nuclease target genomic site may, in some embodiments, be the sequence most frequently bound, or bound with the highest affinity, by a given nuclease.

The term “engineered,” as used herein refers to a protein molecule, a nucleic acid, complex, substance, or entity that has been designed, produced, prepared, synthesized, and/or manufactured by human intervention and an engineered product is a product that does not occur in nature.

The term “effective amount,” as used herein, refers to an amount of a biologically active agent that is sufficient to elicit a desired biological response. For example, in some embodiments, an effective amount of a polynucleotide may refer to the amount of the polynucleotide that is sufficient to induce PU.1 expression after introduction into a target cell. As will be appreciated by the skilled artisan, the effective amount of an agent, e.g., a polynucleotide, a construct, a CRISPR/Cas system, a complex of a protein and a polynucleotide, a polynucleotide, a viral vector, or a non-viral delivery vehicle, may vary depending on various factors as, for example, on the desired biological response, the specific allele, genome, target genomic site, cell, or tissue being targeted, and the agent being used.

The term “delivery vehicle” refers to a construct which is capable of delivering, and, within preferred embodiments expressing, all or a fragment of one or more gene(s) or nucleic acid molecule(s) of interest in a host cell or subject.

The term “fragment of,” or “fragment thereof,” as used herein, refers to a segment (e.g., segments of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 98%, at least about 99%, at least about 99.5%, or at least about 99.9%) of the full length gene(s) or nucleic acid molecule(s) of interest. Representative examples of such delivery vehicles include, but are not limited to, vectors (e.g., viral vectors), nucleic acid expression vectors, naked DNA, naked RNA, and cells (e.g., eukaryotic cells).

The term “homologous,” as used herein is an art-understood term that refers to nucleic acids or polypeptides that are highly related at the level of the nucleotide and/or amino acid sequence. Nucleic acids or polypeptides that are homologous to each other are termed “homologues”. Homology between two sequences can be determined by sequence alignment methods known to those of skill in the art, for instance, using publicly available computer software such as BLAST, ALIGN, or Megalign (DNASTAR) software. In accordance with the invention, two sequences are considered to be homologous if they are at least about 50-60% identical (e.g., at least about 70% identical, at least about 80% identical, at least about 90% identical, at least about 95% identical, at least about 98% identical, at least about 99% identical, at least about 99.5% identical, or at least about 99.9% identical), e.g., share identical residues (e.g., amino acid or nucleic acid residues) in at least about 50-60% of all residues comprised in one or the other sequence, for at least one stretch of at least 20, at least 30, at least 40, at least 50, at least 60, at least 70, at least 80, at least 90, at least 100, at least 120, at least 150, at least 200, at least 250, at least 300, at least 350, at least 400, at least 500, at least 600, at least 700, at least 900, at least 1100, at least 1300, at least 1500, at least 2000, at least 2500, at least 3000, at least 4000, at least 5000, at least 7000, at least 9000, at least 10000, or at least 15000 residues (e.g., amino acids or nucleic acids).

The term “lentiviral vector” refers to a nucleic acid construct derived from a lentivirus which carries, and, within certain embodiments, is capable of directing the expression of, a nucleic acid molecule of interest. Lentiviral vectors can have one or more of the lentiviral wild-type genes deleted in whole or part, but retain functional flanking long-terminal repeat (LTR) sequences (also described below). Functional LTR sequences are necessary for the rescue, replication and packaging of the lentiviral virion. Thus, a lentiviral vector is defined herein to include at least those sequences required in cis for replication and packaging (e.g., functional LTRs) of the virus. The LTRs need not be the wild-type nucleotide sequences, and may be altered, e.g., by the insertion, deletion or substitution of nucleotides, so long as the sequences provide for functional rescue, replication and packaging.

The term “lentiviral vector particle” refers to a recombinant lentivirus which carries at least one gene or nucleotide sequence of interest, which is generally flanked by lentiviral LTRs. The lentivirus may also contain a selectable marker. The recombinant lentivirus is capable of reverse transcribing its genetic material into DNA and incorporating this genetic material into a host cell's DNA upon infection. Lentiviral vector particles may have a lentiviral envelope, a non-lentiviral envelope (e.g., an amphotropic or VSV-G envelope), a chimeric envelope, or a modified envelope (e.g., truncated envelopes or envelopes containing hybrid sequences).

The term “linker” refers to a chemical group or a molecule linking two adjacent molecules or moieties. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is a peptide linker. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker includes the amino acid sequence of any one of (GS)ₙ, (GGS)ₙ, (GGGGS)ₙ, (GGSG)ₙ, (SGGG)ₙ, wherein n is an integer from 1 to 10. In some embodiments, the peptide linker comprises repeats of the tri-peptide Gly-Gly-Ser, e.g., comprising the sequence (GGS)ₙ, wherein n represents at least 1, 2, 3, 4, 5, 6, 7, 8, 9, 10, or more repeats. In some embodiments, the linker comprises the sequence (GGS)₆.

The term “mutation,” as used herein, refers to a substitution, insertion, or deletion of a residue within a sequence, e.g., a nucleic acid or amino acid sequence, with another residue, or a substitution, insertion, or deletion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are discussed in, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, gRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs, such as analogs having chemically modified bases or sugars and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term “percent (%) identity” refers to the percentage of amino acid residues or nucleic acid residues of a candidate sequence, e.g., a LOUP polynucleotide, or fragment thereof, that are identical to the amino acid residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid sequence identity or percent nucleic acid sequence identity of a given candidate sequence to, with, or against a given reference sequence (which can alternatively be phrased as a given candidate sequence that has or includes a certain percent amino acid sequence identity to, with, or against a given reference sequence) is calculated as follows:

100×(fraction of A/B)

where A is the number of amino acid residues or nucleic acid residues scored as identical in the alignment of the candidate sequence and the reference sequence, and where B is the total number of amino acid residues or nucleic acid residues in the reference sequence. In some embodiments where the length of the candidate sequence does not equal to the length of the reference sequence, the percent amino acid sequence identity of the candidate sequence to the reference sequence would not equal to the percent amino acid sequence identity of the reference sequence to the candidate sequence.

Two polynucleotide or polypeptide sequences are said to be “identical” if the sequence of nucleotides or amino acids in the two sequences is the same when aligned for maximum correspondence as described above. Comparisons between two sequences are typically performed by comparing the sequences over a comparison window to identify and compare local regions of sequence similarity. A “comparison window” as used herein, refers to a segment of at least about 15 contiguous positions, about 20 contiguous positions, about 25 contiguous positions, or more (e.g., about 30 to about 75 contiguous positions, or about 40 to about 50 contiguous positions), in which a sequence may be compared to a reference sequence of the same number of contiguous positions after the two sequences are optimally aligned.

As used herein, the term “pharmaceutically acceptable carrier” refers to an excipient or diluent in a pharmaceutical composition. The pharmaceutically acceptable carrier is compatible with the other components of the formulation and not deleterious to the recipient. The pharmaceutically acceptable carrier may impart pharmaceutical stability to the composition (e.g., stability to featured polynucleotides (e.g., polynucleotides including a nucleic acid sequence with at least 20 nucleotides of SEQ ID NO: 1, and variants thereof with at least 85% sequence identity thereto), constructs including the lncRNA (e.g., constructs including a protein linked to a LOUP polynucleotide, and gene editing systems (e.g., a CRISPR/Cas system or CRISPRa)), or may impart another beneficial characteristic (e.g., sustained release characteristics). The nature of the carrier may differ with the mode of administration. For example, for intravenous administration, an aqueous solution carrier is generally used; for oral administration, a solid carrier may be preferred.

As used herein, the term “pharmaceutical composition” refers to a medicinal or pharmaceutical formulation that contains an active agent at a pharmaceutically acceptable purity, as well as one or more excipients and diluents that are suitable for the method of administration and are generally regarded as safe for the recipient according to recognized regulatory standards. The pharmaceutical composition includes pharmaceutically acceptable components that are compatible with, for example, featured polynucleotides (e.g., polynucleotides including a nucleic acid sequence with at least 20 nucleotides of SEQ ID NO: 1, and variants thereof with at least 85% sequence identity thereto), constructs including the lncRNA (e.g., constructs including a protein linked to a LOUP polynucleotide), and gene editing systems (e.g., a CRISPR/Cas system or CRISPRa), and/or nucleic acids encoding the same. The pharmaceutical composition may be in aqueous form, for example, for intravenous or subcutaneous administration, in tablet or capsule form, for example, for oral administration, or in cream for, for example, for topical administration.

The terms “protein” and “peptide” and “polypeptide” are used interchangeably and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The terms “RNA-programmable nuclease” and “RNA-guided nuclease” are used interchangeably and refer to a nuclease of a gene editing system (e.g., a CRISPR/Cas system) that forms a complex with (e.g., specifically binds to or associates with) one or more polynucleotide molecules (e.g., RNA molecules), that are not a target for cleavage, but that direct the RNA-programmable nuclease to a target cleavage site complementary to the spacer sequence of a guide polynucleotide. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target site (e.g., a target genomic site) (e.g., to direct binding of a Cas complex (e.g., a Cas9 complex or dCas9 complex) to the target site); and (2) a domain that binds a Cas nuclease (e.g., a Cas9 or dCas9 protein). In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al. (2012, supra), the entire contents of which are incorporated herein by reference. Still other examples of gRNAs and gRNA structure are provided herein. (see, e.g., the Examples). The gRNA comprises a nucleotide sequence that has a complementary sequence to a target site (e.g., a target genomic site), which mediates binding (e.g., specific binding) of the nuclease/RNA complex to the target site, thereby providing the sequence specificity of the nuclease:RNA complex. In some embodiments, the RNA-programmable nuclease is the (CRISPR-associated system) Cas9 endonuclease, for example Cas9 from Streptococcus pyogenes (see, e.g., Ferretti et al. (2001, supra); Deltcheva et al. (2011, supra); and Jinek et al. (2012, supra)). In some embodiments, the RNA-programmable nuclease is an inactive Cas endonuclease, such as dCas9 described in Qi et al. (Cell, 152(5): 1173-1183, 2013), the entire contents of which are incorporated herein by reference. In some embodiments, the RNA-programmable nuclease (e.g., CRISPR-associated system) is an activating CRISPR system such as described in Konermann et al. (Nature, 517(7536): 583-588, 2015), the entire contents of which are incorporated herein by reference. The term “dCas fusion protein” or “Cas activator”, are used interchangeably to refer to activating CRISPR systems of fusion proteins including a dCas domain linked to one or more transcription factors. Non limiting examples of dCas fusion proteins include dCas9-VP64, dCas9-VPR, dCas9-SAM, dCas9-Scaffold, dCas9-Suntag, dCas9-P300, dCas9-VP160, and VP64-dCas9-BFP-VP64 (Chavez et al. Nat. Methods 13(7): 563-567, 2016; the entire contents of which are incorporated herein by reference).

Because RNA-programmable nucleases (e.g., Cas9 or dCas9) use RNA:DNA hybridization to determine cleavage sites, these proteins are able to cleave or bind to, in principle, any sequence specified by the guide RNA. Methods of using RNA-programmable nucleases, such as Cas9, for site-specific cleavage (e.g., to modify a genome) or gene activation are known in the art (see e.g., Cong et al. (Science 339: 819-823, 2013); Mali et al. (Science 339: 823-826, 2013; Hwang et al. (Nature biotechnology 31: 227-229, 2013); Jinek et al. (eLife 2, e00471, 2013); Dicarlo et al. (Nucleic acids research 10(7):4336-4343, 2013); Jiang et al. (Nature biotechnology 31: 233-239, 2013); and Konermann et al. (supra, 2015); the entire contents of each of which are incorporated herein by reference).

The term “recombine” or “recombination” in the context of a nucleic acid modification (e.g., a genomic modification), is used to refer to the process by which two or more nucleic acid molecules, or two or more regions of a single nucleic acid molecule, are modified by the action of an RNA programmable nuclease (e.g., a Cas9) fusion protein provided herein. Recombination can result in, inter alia, the insertion, inversion, excision or translocation of nucleic acids, e.g., in or between one or more nucleic acid molecules.

The term “subject” refers to an organism, for example, a vertebrate (e.g., a mammal, bird, reptile, amphibian, and fish). In some embodiments, the subject is a human. In some embodiments, the subject is a non-human mammal (e.g., a non-human primate). In some embodiments, the subject is a sheep, a goat, a bovine (e.g., a cow, bull, or ox), a rodent, a cat, a dog, an insect (e.g., a fly), or a nematode. In some embodiments, the subject is a research animal. In some embodiments, the subject is genetically engineered, e.g., a genetically engineered non-human subject. The subject may be of either sex and at any stage of development.

The terms “target nucleic acid” and “target genome” and “endogenous DNA” as used herein in the context of nucleases, refer to a nucleic acid molecule (e.g., a nucleic acid molecule of a genome, such as a nucleic acid molecule of a chromosome (e.g., a gene)), that comprises at least one target site (e.g., a target genomic site) of an RNA-programmable nuclease. In some embodiments, the target nucleic acid(s) comprises at least two, at least three, or at least four target genomic sites.

The term “target site” refers to a sequence within a nucleic acid molecule that is bound by a nuclease (e.g., Cas or a dCas fusion protein described herein). A “target genomic site” refers to a sequence within the genome of a subject (e.g., a site in a chromosome, such as within a gene). A target site or target genomic site may be single-stranded or double-stranded. In the context of RNA-guided (e.g., RNA-programmable) nucleases (e.g., a Cas or dCas nuclease), a target genomic site typically comprises a nucleotide sequence that is complementary to the gRNA(s) of the RNA-programmable nuclease and a protospacer adjacent motif (PAM) at the 3′ end adjacent to the gRNA-complementary sequence(s) on the non-target strand. In some embodiments, such as those involving Cas nucleases, a target site or target genomic site can encompass the particular sequences to which Cas monomers bind and/or the intervening sequence between the bound monomers that are cleaved by the Cas nuclease domain. For the RNA-guided nuclease Cas (or gRNA-binding domain thereof) and dCas described herein, the target site or target genomic site may be, in some embodiments, 17-25 base pairs plus a 3 base pair PAM (e.g., NNN, wherein N independently represents any nucleotide). Typically, the first nucleotide of a PAM can be any nucleotide, while the two downstream nucleotides are specified depending on the specific RNA-guided nuclease. Exemplary PAM sites for RNA-guided nucleases, such as Cas9, are known to those of skill in the art and include, without limitation, NGG (SEQ ID NO: 11), NAG (SEQ ID NO: 12), and NGNG (SEQ ID NO: 16), wherein N independently represents any nucleotide. In addition, Cas9 nucleases from different species (e.g., S. thermophilus instead of S. pyogenes) recognize a PAM that comprises the sequence NGGNG (SEQ ID NO: 25). Additional PAM sequences are known, including, but not limited to, NNAGAAW (SEQ ID NO: 24) and NAAR (SEQ ID NO: 27) wherein W independently represents A or T, and wherein R independently represents A or G (see, e.g., Esvelt and Wang (Molecular Systems Biology, 9:641, 2013), the entire contents of which are incorporated herein by reference). In some aspects, the target site or target genomic site of an RNA-guided nuclease, such as, e.g., Cas9, may comprise the structure [N_{Z}]-[PAM], where each N is, independently, any nucleotide, and z is an integer between 1 and 50, inclusive. In some embodiments, z, which is the number of N nucleotides, is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50. In some embodiments, z is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, z is 20.

As used herein, the term “therapeutically effective amount” refers to an amount, e.g., a pharmaceutical dose of a composition described herein (e.g., a composition containing featured polynucleotides (e.g., polynucleotides including a nucleic acid sequence with at least 20 nucleotides of SEQ ID NO: 1, and variants thereof with at least 85% sequence identity thereto), constructs including the lncRNA (e.g., constructs including a protein linked to a LOUP polynucleotide), and gene editing systems (e.g., a CRISPR/Cas system or CRISPRa), and/or nucleic acids encoding the same as described herein), effective in inducing a desired biological effect in a subject or in treating a subject with a medical condition or disorder described herein (e.g., cancer (e.g., a cancer associated with PU.1 expression (e.g., acute myeloid leukemia, liver cancer, or myeloma))). It is also to be understood herein that a “therapeutically effective amount” may be interpreted as an amount giving a desired therapeutic effect, either taken in one dose or in any dosage or route, taken alone or in combination with other therapeutic agents.

As used herein, the terms “treatment” and “treating” refer to reducing or ameliorating a medical condition (e.g., a disease or disorder associated with PU.1 expression (e.g., a cancer (e.g., acute myeloid leukemia, liver cancer, or myeloma)), Alzheimer's disease, or asthma) and/or symptoms associated therewith. It will be appreciated that, although not precluded, treating a medical condition does not require that the disorder or symptoms associated therewith be completely eliminated. Reducing or decreasing the side effects of a medical condition, such as those described herein, or the risk or progression of the medical condition, may be relative to a subject who did not receive treatment, e.g., a control, a baseline, or a known control level or measurement. The reduction or decrease may be, e.g., by about 1%, 2%, 3%, 4%, 5%, 6%, 7%, 8%, 9%, 10%, 20%, 30%, 40%, 50%, 60%, 70%, 80%, 90%, 95%, 97%, 99%, or about 100% relative to the subject who did not receive treatment or the control, baseline, or known control level or measurement, or may be a reduction in the number of days during which the subject experiences the medical condition or associated symptoms (e.g., a reduction of 1-30 days, 2-12 months, 2-5 years, or 6-12 years). As defined herein, a therapeutically effective amount of a pharmaceutical composition of the present disclosure may be readily determined by one of ordinary skill by routine methods known in the art. Dosage regimen may be adjusted to provide the optimum therapeutic response.

The term “substantially” used herein allows for deviations from the descriptor that do not negatively impact the intended purpose. Descriptive terms may be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

Wherever any of the phrases “such as,” “for example,” “including” and the like are used herein, the phrase “and without limitation” is understood to follow unless explicitly stated otherwise. Similarly, “an example,” “exemplary,” and the like are understood to be non-limiting.

The term “vector” refers to a polynucleotide comprising one or more recombinant polynucleotides described herein, e.g., those encoding a featured polynucleotide (e.g., a polynucleotide including a nucleic acid sequence with at least 20 nucleotides of SEQ ID NO: 1, and variants thereof with at least 85% sequence identity thereto), a construct including the lncRNA (e.g., constructs including a protein linked to a LOUP polynucleotide, and a gene editing system (e.g., a CRISPR/Cas system or CRISPRa) described herein. Vectors include, but are not limited to, plasmids, viral vectors, cosmids, artificial chromosomes, and phagemids. Typically, a vector is able to replicate in a host cell and can be further characterized by one or more endonuclease restriction sites at which the vector may be cut and into which a desired nucleic acid molecule may be inserted. Vectors may contain one or more marker sequences suitable for use in the identification and/or selection of cells which have or have not been transformed or genome-modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., p-galactosidase, alkaline phosphatase, or luciferase), and genes which visibly affect the phenotype of transformed or transfected cells, hosts, colonies, or plaques. Any vector suitable for the transformation of a host cell (e.g., E. coli, mammalian cells such as CHO cell, insect cells, etc.) as embraced by the present invention, for example, vectors belonging to the pUC series, pGEM series, pET series, pBAD series, pTET series, or pGEX series. In some embodiments, the vector is suitable for transforming a host cell for recombinant protein production. Methods for selecting and engineering vectors and host cells for expressing proteins (e.g., those provided herein), transforming cells, and expressing/purifying recombinant proteins are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

### BRIEF DESCRIPTION OF THE DRAWINGS

FIGS. 1A-1E show screening of gene loci exhibiting concurrent RUNX1-RNA and -DNA interactions in THP-1 cells. FIGS. 1A and 1B are pie chart representations of proportions of RUNX1 fRIP-seq peaks and RUNX1 ChIP-seq peaks in coding and noncoding gene families. ChIP-seq data were published under the Gene Expression Omnibus (GEO) accession number: GSE79899. FIG. 1C is a Venn diagram presentation of intersecting RUNX1 fRIP-seq, RUNX1 ChIP-seq gene lists and the myeloid gene list. FIG. 1D is an image showing a gene track view of the PU.1 locus including the upstream region (highlighted in blue). Shown are fRIP-seq tracks (Input, IgG and RUNX1) and RUNX1 ChIP-seq track (GSM2108052). Data were integrated in the UCSC genome browser. FIG. 1E is an image showing RUNX1 fRIP-qPCR confirmation. Left panel: Location of three PCR amplicons (#1, #2, #3). Right panel: bar graph showing the enrichment of RNAs captured by anti-RUNX1 antibody and IgG control at three amplicons relative to input.

FIGS. 2A-2G show the identification of gene loci exhibiting concurrent RUNX1-RNA and -DNA interactions. FIG. 2A is diagram showing the workflow of RUNX1-fRIP procedure. FIG. 2B is an image showing an immunoblot detection of RUNX1 and actin immunoprecipitated from THP-1 cell lysate using anti-RUNX1 antibody and IgG control. FIG. 2C shows chromatographs of bioanalyzer analysis of RNAs captured by anti-RUNX1 antibody and IgG control plus input RNAs. FIG. 2D is a diagram of an analysis flowchart of RUNX1 fRIP-seq and ChIP-seq analyses. FIGS. 2E and 2F are pie charts showing distribution of RUNX1 fRIP-seq peaks and RUNX1 ChIP-seq peaks at different genomic locations. FIG. 2G shows images of the myeloid gene loci having both RUNX1 fRIP peaks and RUNX1 ChIP-seq peaks.

FIGS. 3A-3E show the characterization of lncRNA LOUP. FIG. 3A shows a gene track view of the genomic region encompassing the PU.1 locus. RNA-seq tracks include THP-1, HL60, primary monocytes, and Jurkat. DNAse-seq and ChIP-seq are overlay tracks of monocyte and myeloid cell lines. These data were processed from published data in GEO. CAGE track was imported from the FANTOM5 project. #1, #2 and arrows point to locations of the RNA peaks. FIG. 3B shows the results of RT-PCR analysis of LOUP's transcript features. First-strand cDNAs were generated from HL-60 total RNA using a primer that does not anneal to the PU.1 locus (unrelated), random hexamers, oligo dT, and strand-specific primers (Anti-sense and Sense). FIG. 3C shows images of northern blot analysis of LOUP. polyA− and polyA+ RNA fractions were isolated from U937 and Jurkat cells. Top panel: schematic of probe location spanning exon junction (E1 and E2a). Middle panel: Northern blot detection of LOUP's major and minor transcripts. Lower panel: RNA gel showing relative distance between 28S and 18S rRNAs. FIG. 3D is a graph depicting the qRT-PCR analysis of LOUP levels in polyA− and polyA+ RNA fractions isolated from HL-60 cells. FIG. 3E is a graph depicting the calculation of LOUP transcript per cell by RT-qPCR. LOUP RNA standard curve was generated by in vitro transcription. Error bars indicate SD. ***p<0.001.

FIGS. 4A-4I show transcript maps and molecular features of LOUP. FIG. 4A are images depicting RT-PCR confirmation of exon-exon junction of LOUP; Upper panel: Schematics of the PCR amplicon and primer locations. Lower panels: DNA sequencing of PCR products from human (HL-60) and murine (RAW264.7) cells. FIG. 4B is a diagram depicting the workflow of 5′ end mapping by P5-linker ligation method. FIG. 4C show images of P5-linker ligation assay for determining the 5′ end of LOUP transcript. Upper panel: DNA sequencing analysis showing locations of P5-primer, P5-splinkerette and transcription start site (TSS). Lower panel: Schematic diagram of the PU.1 locus. Shown are the URE element with two homology regions H1 and H2. FIG. 4D is a schematic diagram showing relative genomic location of LOUP and two neighbor genes PU.1 and SLC39A13 (top) and splicing pattern of LOUP (bottom). E1: Exon 1, E2: Exon 2, E2a and E2b are exons derived from an additional splicing event within Exon 2. Exon boundaries were mapped by 3′RACE and RT-PCR. FIG. 4E is a graph depicting the results from a PhyloCSF analysis of LOUP and other known coding and noncoding genes. Shown are coding potential scores. FIG. 4F are bar graphs depicting RT-qPCR analysis of Loup in subcellular fractions isolated from RAW264.7 cells. Fraction enrichment controls include Malat1 (chromatin) and Rps18 (cytoplasm) (West et al., Mol. Cell 55: 791-802 2014). FIG. 4G is a bar graph showing qRT-PCR analysis of fraction enrichment controls including MALAT1 (polyA+) and RPPH1 (polyA−) (right panel). FIG. 4H shows a schematic diagram and graphs depicting the measurement of transcript numbers per HL-60 cell. Upper panel: Schematic diagram of amplified amplicons showing primer locations for non-spliced LOUP (FW2-RV) and spliced LOUP (FW1-RV). Lower panels: RT-qPCR with RNA standard curve for spliced and non-spliced forms. FIG. 4I are bar graphs showing RT-qPCR analysis of LOUPforms in the nucleus (left panel) and fraction enrichment controls include MALAT1 (nucleoplasm) and RPS18 (cytoplasm) (right panel). Error bars indicate SD.

FIGS. 5A-5E show bar graphs presenting expression profiles of LOUP and PU.1 in normal tissues and cell lineages. FIG. 5A-5B are bar graphs showing transcript profiles of LOUP (FIG. 5A) and PU.1 (FIG. 5B) in human tissues. Shown are transcript counts from the Illumina Body Map RNA-seq data dataset (AEArrayExpress: E-MTAB-513). FIG. 5C is a bar graph showing the proportion of cell lineages corresponding to LOUP and PU.1 transcript levels. Myeloid: includes mono, macrophage and granulocyte, T_{CD4+}: T helper cell, T_{CD8+}: Cytotoxic T cell, T_{reg}: Regulatory T cell, B: B lymphocyte, Plas: Plasma cell, NK: Natural killer cell, DC: Dendritic cell, Ery: Erythrocyte, Meg: Megakaryocyte. FIGS. 5D and 5E are bar graphs showing results from RT-qPCR analysis of Loup (FIG. 5D) and Pu.1 (FIG. 5E) RNA levels in murine hematopoietic stem, progenitor and mature (myeloid) cell populations. LT-HSC: long-term hematopoietic stem cells, ST-HSC: short-term hematopoietic stem cells, CMP: common myeloid progenitors, MEP: megakaryocyte-erythroid progenitors, LMPP: lymphoid-primed multipotent progenitors, GMP: granulocyte-macrophage progenitors, myeloid cells. Data are shown relative to LT-HSC. Error bars indicate SD.

FIGS. 6A-6G depict gene expression profiles in normal tissues and cell lineages. FIGS. 6A and 6B are bar graphs showing transcript profiles of SLC39A13 and RUNX1 in human tissues from the Illumina Body Map dataset. FIG. 6C is a k-nearest neighbor graph depicting the results from a SRING plot analysis of the 10× Genomic scRNA-seq dataset showing color-coded definitive blood lineages using Blueprint-Encode annotation (Aran et al., 2019). FIGS. 6D-6F are graphs showing transcript profiles of LOUP, PU.1 and RUNX1, respectively, in blood cell lineages of the 10× Genomic scRNA-seq dataset. Each dot on the graph represents an individual cell. FIG. 6G is a bar graph depicting the results of a GO analysis for enrichment of biological processes using a list of genes upregulated in LOUP^{high}/pU.1^{high} cells as compared to LOUP^{low}/PU.1^{high} cells. Error bars indicate SD.

FIGS. 7A-7F show LOUP and PU.1 expression correlation. FIG. 7A is a schematic diagram of the upstream genomic region of the PU.1 locus. Shown are sgRNA-binding sites (#D1 and #D2) for LOUP depletion using CRISPR/Cas9 technology. FIGS. 7B and 7C are bar graphs showing results of RT-qPCR expression analysis for LOUP (FIG. 7B) and PU.1 (FIG. 7C) in non-targeting (N) and LOUP-targeting (L) U937 cell clones. Data are shown relative to control. FIG. 7D are bar graphs showing RT-qPCR expression analysis of LOUP (left panel) and PU.1 (right panel) in K562 cells transfected with LOUP cDNA or empty vector (EV) by electroporation. FIG. 7E is a schematic diagram of the LOUP promoter region showing sgRNA-binding sites (#A1 and #A2) for LOUP induction. Distance from the TIS of LOUP is indicated in bp. FIG. 7F are bar graphs depicting RT-qPCR expression analysis of LOUP (left panel) and of PU.1 (right panel) in K562 dCas9-VP64-stable cells infected with LOUP-targeting (#A1 and #A2) or non-targeting (control) sgRNAs. Error bars indicate SD. **p<0.01; ****p<0.0001.

FIGS. 8A-8H present the effects of LOUP's loss- and gain-of-expression. FIG. 8A is a schematic strategy for LOUP depletion. Included is a FACS sorting scheme for isolation of cells expressing both mCherry (Cas9) and eGFP (sgRNAs). FIGS. 8B and 8C present the results from an Interference of CRISPR Edits (ICE) analyses for indel composition and frequency of CRISPR/Cas9 cell clones. Top panels: Trace file segments of amplified genomic regions surrounding sgRNA-binding sites (#D1 and #D2 LOUPsgRNAs) in edited (upper panel) and the control (lower panel) samples. Dotted red underline: Protospacer adjacent motif (PAM) sequence. Solid black underline: guide sequences. Expected cut sites are denoted as vertical dotted lines. Bottom-left panel: Indel efficiency analysis. Bottom-right panel: Indel distribution analysis. Dashed lines indicate deletion length. FIG. 8D is an image depicting genomic PCR and Sanger sequencing confirmation of U937 cell clones with LOUP homozygous indels (L2a and L2b) and control (N1). FIG. 8E is a chromatograph showing the results of a fluorescence-activated cell sorting (FACS) analysis of CD11b myeloid marker in U937 cell clones with LOUP homozygous indels (L2a and L2b) and control (N1 and N2) using PACBLUE-conjugated CD11b antibody. FIGS. 8F-8H are bar graphs depicting qRT-PCR analysis of LOUP and PU.1 RNA levels in K562 (8F), Jurkat (8G), and Kasumi-1 (8H) cells stably carrying empty vector or LOUP cDNA via lentiviral transduction. Error bars indicate SD. **p<0.01; ***p<0.001, n.s: not significant.

FIGS. 9A-9D present 3C and ChIRP assays measuring LOUP's effects on chromatin looping. FIG. 9A is a schematic diagram illustrating potential 3C interactions between the URE and genomic viewpoints surrounding the PU.1 locus including restriction recognition sites of ApoI that was used in the assay. FIG. 9B is a bar graph depicting the results from a 3C-qPCR TaqMan probe-based assay comparing crosslinking frequencies at chromatin viewpoints. The U937 cell clone L2a, carrying LOUP homozygous indels that does not alter recognition pattern of ApoI, was used to compare with non-targeting control (sgControl, N1). n.d.: not detectable. FIG. 9C is a bar graph depicting the results from RT-qPCR evaluating levels of LOUP RNA and control GAPDH captured by biotinylated LOUP-tiling and LacZ-tiling probes. FIG. 9D is a bar graph showing the results from a ChIRP assay assessing LOUP occupancies at the URE, the PrPr, and ACTB promoter. LOUP-tiling oligos were used to capture endogenous LOUP in U937 cells. LacZ-tiling oligos were used as negative control. Error bars indicate SD; *p<0.05; ****p<0.0001, n.s: not significant.

FIGS. 10A-10G shows that LOUP cooperates with RUNX1 to facilitate URE-PrPr interaction. FIG. 10A is a gene track view of the ˜26 kb region encompassing the URE and the PrPr. Shown are RUNX1 ChIP-seq tracks of CD34⁺ cells from healthy donors (GSM1097884), AML patient with FLT3-ITD AML (GSM1581788) non-t(8;21) AML patient (GSM722708) (top panel). Schematics showing corresponding genomic locations of LOUP and 5′ part of PU.1 (bottom panel). FIG. 10B are images depicting immunoblots from a DNA affinity precipitation (DNAP) assay showing binding of RUNX1 to the RUNX1-binding motifs at the URE and the PrPr. Proteins captured by biotinylated DNA oligos (wt: wildtype oligo containing RUNX1-binding motif, mt: oligo with mutated RUNX1-binding motif) in U937 nuclear lysate were detected by immunoblot. FIG. 10C is a bar graph showing ChIP-qPCR analysis of RUNX1 occupancy at the URE and the PrPr. LOUP-depleted U937 (sgLOUP, L2a) and control (sgControl, N1) clones were used. PCR amplicons include URE (contains known RUNX1-binding motif at the URE), PrPr (contains putative RUNX1-binding motif at the PrPr), and GENE DESERT (a genome region that is devoid of protein-coding genes). FIG. 10D is a schematic depicting RNAP analysis of RUNX1-LOUP interaction. Upper panel: Schematic diagram of LOUP showing relative position of the RR. Underneath arrows illustrate direction and relative lengths of in vitro-transcribed and biotin-labeled LOUPfragments (Bead: no RNA control, EGFP: EGFP mRNA control, AS: full-length antisense control, S: full-length sense, and RR). Lower panel: LOUP fragments were incubated with U937 nuclear lysates. Retrieved proteins were identified by immunoblot. FIG. 10E is a schematic diagram of the RR showing predicted binding regions R1 and R2. FIGS. 10F and 10G are images of immunoblots showing RNAP binding analysis of R1 and R2 with recombinant full-length and Runt domain of RUNX1. In vitro-transcribed and biotin-labeled RNAs includes R1-AS (R1 antisense control), R1-S(R1 sense), and R2-S (R2 sense). Vertical line demarcates where an unrelated lane was removed. Error bars indicate SD.

FIG. 11A is an image of an immunoblot of RUNX1 and control proteins in nuclear and cytosol fractions from U937 cells.

FIG. 11B is a nucleotide identity plot generated from alignment of LOUP to itself using discontinuous megablast algorithm from BLAST (blast.ncbi.nlm.nih.gov/). Boxed area depicts a repetitive region of 670 bp.

FIG. 11C is a schematic diagram of the RR illustrating three TE variants (L1 PB4, AluJb and AluSx) identified by Repeatmasker software (Smit, 2013).

FIG. 11D is a graph depicting the In silico prediction of RR-RUNX1 interaction by catRAPID Fragments algorithm. R1 and R2: two regions with high interaction scores.

FIG. 12 is a schematic diagram illustrating how LOUP coordinates with RUNX1 to modulate chromatin looping

### DETAILED DESCRIPTION

Described herein are long non-coding RNA (e.g., LOUP RNA), polynucleotides encoding the lncRNA, vectors (e.g., viral vectors) containing polynucleotides encoding the lncRNA, constructs containing LOUP, methods of delivering LOUP, methods of increasing or decreasing LOUP expression using a gene editing system (e.g., a CRISPR/Cas system or CRISPRa), methods of altering PU.1 expression, methods of treating a disease (e.g., cancer (e.g., PU.1 associated cancer (e.g., AML, liver cancer, and myeloma)), Alzheimer's disease, or asthma), and methods of diagnosing treatment responsiveness (e.g., ATRA treatment) in a subject with cancer (e.g., AML, liver disease, or myeloma).

We discovered that an uncharacterized myeloid-specific lncRNA, termed “Long noncoding RNA Originating from the URE of PU.1”, or LOUP, induces gene-specific long-range transcription by modulating enhancer docking to a specific proximal promoter. LOUP is a product of unidirectional transcription, and undergoes splicing and polyadenylation, thereby exhibiting all the features of a 1d-eRNA. At single-cell resolution, LOUP and PU.1 expression is stringently associated with myeloid lineage identity. Both gain- and loss-of-function experiments demonstrated a LOUP-dependent expression of PU.1. We further discovered that LOUP associates with chromatin and induces interaction between the URE and the PrPr, resulting in the formation of an active chromatin loop at the PU.1 locus. Finally, we showed that LOUP recruits RUNX1 to its DNA-binding motifs at both the URE and the PrPr via a region embedded with transposable element (TE) variants. Collectively, these findings reveal an unanticipated role of a cell type-specific and TE-embedded 1d-eRNA in mediating gene-specific long-range transcription by cooperating with a ubiquitously expressed transcription factor.

The present disclosure relates to long non-coding RNA (e.g., LOUP RNA), polynucleotides encoding the lncRNA (e.g., a polynucleotide having at least 20 nucleotides of SEQ ID NO: 1), vectors (e.g., viral vectors) including polynucleotides encoding the lncRNA (or at least, e.g., 20 nucleotides or more, encoding the lncRNA), constructs including the lncRNA (e.g., constructs including a protein linked to a LOUP polynucleotide), a gene editing system (e.g., a CRISPR/Cas system or CRISPRa) for regulating PU.1 expression, polynucleotides encoding the gene editing system, vectors (e.g., viral vectors) including polynucleotides encoding the gene editing system and compositions including the same, and cells containing one or more of these compositions. The compositions disclosed herein can be used in methods of diagnosing, treating, and/or preventing conditions associated with PU.1 expression (e.g., cancer (e.g., AML, liver cancer, or myeloma), Alzheimer's disease, or asthma).

Polynucleotides

Featured polynucleotides include any nucleotide capable of inducing PU.1 expression. In some embodiments, the polynucleotide includes a binding region for Runt-related transcription factor 1 (RUNX1) protein, or fragment thereof. For example, the polynucleotide may include a nucleic acid sequence with at least about 20 nucleotides (e.g., at least about 25, at least about 40, at least about 60, at least about 80, at least about 100, at least about 150, at least about 300, at least about 500, at least about 900, at least about 1300, at least about 1700, at least about 2000, at least about 2300, at least about 2350, or at least about 2375) of SEQ ID NO: 1 and variants thereof with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. In some instances, the polynucleotide may include a nucleic acid sequence with between about 20 nucleotides and about 2380 nucleotides (e.g., between about 20 and about 100, between about 70 and about 300, between about 200 and about 500, between about 400 and about 800, between about 700 and about 1200, between about 1100 and about 1600, between about 1500 and about 2000, or between about 1900 and about 2380) or SEQ ID NO: 1, or variants thereof with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. In particular, the polynucleotide contains one or more transposable elements (TEs) (e.g., 1, 2, 3, 4, 5, 6, 7, 8, 9, or more TEs). The one or more transposable elements have a nucleic acid sequence of any one of SEQ ID NOs: 2-4 or a variant thereof with at least 85% (e.g., (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. The TE(s) of the polynucleotide may have a minimum length of at least about 50 nucleotides of the nucleotides of any one of SEQ ID NO: 2 or 3 (e.g., at least about 60, 70, 80, 90, 100, 110, 120, 130, 140, 150, 160, 170, or 180 or more nucleotides of SEQ ID NO: 2 or 3) or a variant thereof. In some embodiments, the polynucleotide includes two or three of the TEs or a variant thereof. For example, the polynucleotide includes a first TE of SEQ ID NO: 2, or a variant thereof, and a second TE of SEQ ID NO: 3 or 4, or a variant thereof (e.g., the polynucleotide includes TEs of SEQ ID NOs: 2 and 3, or variants thereof, or TEs of SEQ ID NOs: 2 and 4, or variants thereof). The polynucleotide may also include a first TE of SEQ ID NO: 3 and a second TE of SEQ ID NO: 4, or variants thereof.

Constructs

Featured constructs include a RUNX1 protein, or fragment thereof, conjugated to any polynucleotide capable of inducing PU.1 expression. In some embodiments, the RUNX1 protein, or fragment thereof, is bound (e.g., covalently bound) to any polynucleotide capable of inducing PU.1 expression. In some embodiments the constructs have the structure:

R-L-P (I) or P-L-R (II),

wherein R is the RUNX1 protein or fragment thereof;

P is the polynucleotide; and

L is a linker.

In some embodiments, the construct has the structure R-L-P (I). In other embodiments, the construct has the structure P-L-R (II). The RUNX1 protein may have at least 100 amino acids of SEQ ID NO: 5, or a variant thereof with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. The RUNX1 protein may have at least one binding site (e.g., one, two, three, four, five, or more binding sites) for at least one polynucleotide regulatory element of PU.1 (e.g., at least one, two, three, four, five, or more regulatory elements of PU.1). In certain embodiments, the at least one PU.1 regulatory element has at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to, or the sequence of, SEQ ID NO: 6. In some embodiments, the at least one PU.1 regulatory element is an upstream regulatory element (URE) and/or a proximal promoter region (PrPr). In some embodiments, the at least one PU.1 regulatory element is an upstream regulatory element (URE). In some instances, the URE sequence has at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to the sequence of SEQ ID NO: 6. In some instances, the URE has the sequence of SEQ ID NO: 6. In other embodiments, the at least one PU.1 regulatory element is a proximal promoter region (PrPr). In some instances, the PrPr sequence has at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity to the sequence of SEQ ID NO: 7. In some instances, the PrPr sequence has the sequence of SEQ ID NO: 7.

The polynucleotide of the construct may have a nucleic acid sequence with at least about 20 nucleotides (e.g., at least about 25, at least about 40, at least about 60, at least about 80, at least about 100, at least about 150, at least about 300, at least about 500, at least about 900, at least about 1300, at least about 1700, at least about 2000, at least about 2300, at least about 2350, or at least about 2375) of SEQ ID NO: 1 and variants thereof with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. For example, the polynucleotide may include a nucleic acid sequence with between about 20 nucleotides and about 2380 nucleotides (e.g., between about 20 and about 100, between about 70 and about 300, between about 200 and about 500, between about 400 and about 800, between about 700 and about 1200, between about 1100 and about 1600, between about 1500 and about 2000, or between about 1900 and about 2380) of SEQ ID NO: 1, or a variant thereof with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. In particular, the polynucleotide contains one or more transposable elements (TEs) (e.g., 1, 2, 3, 4, 5, 6, 7, 8, 9, or more TEs). The one or more transposable elements have a nucleic acid sequence of any one of SEQ ID NOs: 2-4 or a variant thereof with at least 85% (e.g., (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. The TE(s) of the polynucleotide may have a minimum length of at least about 50 nucleotides of the nucleotides of any one of SEQ ID NO: 2 or 3 (e.g., at least about 60, 70, 80, 90, 100, 110, 120, 130, 140, 150, 160, 170, or 180 or more nucleotides of SEQ ID NO: 2 or 3) or a variant thereof with at least 85% (e.g., (e.g., at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity thereto. In some embodiments, the polynucleotide includes two or three of the TEs or a variant thereof. For example, the polynucleotide includes a first TE of SEQ ID NO: 2, or a variant thereof, and a second TE of SEQ ID NO: 3 or 4, or a variant thereof (e.g., the polynucleotide includes TEs of SEQ ID NOs: 2 and 3, or variants thereof, or TEs of SEQ ID NOs: 2 and 4, or variants thereof). The polynucleotide may also include a first TE of SEQ ID NO: 3 and a second TE of SEQ ID NO: 4, or variants thereof.

CRISPR/Cas

CRISPR/Cas systems may be used to alter the expression profile of anti-tumor proliferating gene PU.1. The CRISPR/Cas system may be designed to decrease the expression of LOUP. Alternatively, a CRISPR activating (CRISPRa) system may be used to increase the expression of LOUP, thereby increasing PU.1 expression.

The CRISPR/Cas system derives from a prokaryotic immune system that confers resistance to foreign genetic elements, such as those present within plasmids and phages. CRISPR itself comprises a family of DNA sequences in bacteria, which encode small segments of DNA from viruses that have previously been exposed to the bacterium. These DNA segments are used by the bacterium to detect and destroy DNA from similar viruses during subsequent attacks. In a palindromic repeat, the sequence of nucleotides is the same in both directions. Each repetition is followed by short segments of spacer DNA from previous exposures to foreign DNA (e.g., a virus or plasmid). Small clusters of Cas (CRISPR-associated system) genes are located next to CRISPR sequences. These observations form the basis of the CRISPR/Cas system in eukaryotic cells that allows for genome editing. By delivering an RNA programmable nuclease (e.g., a Cas9 nuclease) with one or more guide polynucleotides (e.g., one or more gRNAs) into a cell, the cell's genome can be edited at desired locations (e.g., coding or non-coding regions of a genome of a host cell), allowing an existing gene(s) to be modified and/or removed and/or new gene(s) to be added (e.g., a functional version of a defective gene). The Cas9-gRNA complex corresponds with the type II CRISPR/Cas RNA complex.

A number of bacteria express Cas9 protein variants that can be used in the featured methods (see, e.g., Tables 1 and 2). The Cas9 from Streptococcus pyogenes is presently the most commonly used. Several other Cas9 proteins have high levels of sequence identity with the S. pyogenes Cas9 and use the same guide RNAs. Still, others are more diverse, use different gRNAs, and recognize different PAM sequences as well (the 2-5 nucleotide sequence specified by the protein which is adjacent to the sequence specified by the RNA; see, e.g., Table 2). Chylinski et al. (RNA Biol. 10(5): 726-737, 2013) classified Cas9 proteins from a large group of bacteria, and a large number of Cas9 proteins are described herein. Additional Cas9 proteins that can be used in the featured gene editing system are described in, e.g., Esvelt et al. (Nat Methods 10(11): 1116-21, 2013) and Fonfara et al. (Nucleic Acids Res. 42(4): 2577-2590, 2013); incorporated herein by reference.

Cas molecules from a variety of species can be incorporated into the methods (e.g., the methods of treating a medical condition (e.g., a medical condition associated with PU.1 expression), compositions, and kits described herein. While the S. pyogenes Cas9 molecule is the subject of much of the disclosure herein, Cas9 molecules of, derived from, or based on the Cas9 proteins of other species listed herein can be used as well. In other words, while much of the description herein refers to S. pyogenes Cas9 molecules, Cas9 molecules from the other species can replace them. Such species include those set forth in the following Table 1:

TABLE 1
Exemplary Cas9 nucleases

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp- B510 |
| 92109262 | Nitrobacter hamburgensis X14 |
| 148255343 | Bradyrhizobium sp- BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

TABLE 2
Exemplary Cas nucleases and their associated PAM sequence

| | Class and | PAM | Target | | SEQ |
|---|---|---|---|---|---|
| Species/Variant of Cas | Type | Sequence | Length | | ID NO |
| SpCas9 | Class II type II | 3′ NGG | 20 | nt | 11 |
| Streptococcus pyogenes | | | | | |
| (SP) | | | | | |
| SpCas9 | Class II type II | 3′ NGG | 20 | nt | 11 |
| D1135E variant | | (3′NAG reduced | | | 12 |
| | | binding) | | | |
| SpCas9 | Class II type II | 3′ NGCG | 20 | nt | 13 |
| VRER variant | | | | | |
| SpCas9 | Class II type II | 3′ NGAG | 20 | nt | 14 |
| EQR variant | | | | | |
| SpCas9 | Class II type II | 3′ NGAN; or | 20 | nt | 15 |
| VQR variant | | 3′ NGNG | | | 16 |
| SaCas9 | Class II type II | 3′ NNGRRT or | 20 to 24 | nt | 17 |
| Staphylococcus aureus | | 3′ NNGRR(N) | | | 18 |
| (SA) | | | | | |
| SaCas9 | Class II type II | 3′ NNNRRT | 21 | nt | 19 |
| Staphylococcus aureus | | | | | |
| KKH variant | | | | | |
| Cas12a: | Class Il type V | 5′ TTTV | 23, 24 | nt | 20 |
| Acidaminococcus sp. | | | | | |
| (AsCpf1) and | | | | | |
| Lachnospiraceae | | | | | |
| bacterium (LbCpf1) | | | | | |
| Cas12a | Class II type V | 5′ TYCV | 20 | nt | 21 |
| AsCpf1 RR variant | | | | | |
| Cas12a | Class II type V | 5′ TYCV | 20 | nt | 21 |
| LbCpf1 RR variant | | | | | |
| Cas12a | Class II type V | 5′ TATV | 20 | nt | 22 |
| AsCpf1 RVR variant | | | | | |
| NmCas9 | Class II type II | 3′ NNNNGATT | 23, 24 | nt | 23 |
| Neisseria meningitidis | | | | | |
| (NM) | | | | | |
| StCas9 | Class II type II | 3′ NNAGAAW | 19 to 20 | nt | 24 |
| Streptococcus | | | | | |
| thermophilus1 (ST) | | | | | |
| StCas9 | Class II type II | 3′ NGGNG | 19 | nt | 25 |
| Streptococcus | | | | | |
| thermophilus3 | | | | | |
| TdCas9 | Class II type II | 3′ NAAAAC | 20 | nt | 26 |
| Treponema denticola | | | | | |
| (TD) | | | | | |
| Cas13a (C2c2) | Class II type VI | N/A | N/A | | |
| Leptotrichia buccalis | | | | | |
| Cas13a (C2c2) | Class II type VI | N/A | N/A | | |
| Leptotrichia shahii | | | | | |

N/A - Cas13a have not been used in mammalian cells. The functional target length and PAM site remains unclear. For PAM sites: N can be any base; R can be A or G; V can be A, C, or G; W can be A or T; and Y can be C or T.

By way of example and not limitation, the methods described herein can include the use of any of the Cas proteins from Tables 1 and 2 and their corresponding guide polynucleotide(s) (e.g., guide RNA(s)) or other compatible guide RNAs. As an example, and not intended to be limiting in any way, the Cas9 from Streptococcus thermophilus LMD-9 CRISPR1 system has been shown to function in human cells (see, e.g., Cong et al. (2013, supra)). Cas9 orthologs from N. meningitides, which are described, e.g., in Hou et al. (Proc Natl Acad Sci USA. 110(39): 15644-9, 2013) and Esvelt et al. (2013, supra), can also be used in the compositions and methods described herein.

Guide Polynucleotides

The featured CRISPR/Cas protein complexes of the methods and compositions can be guided to a target site (e.g., a target genomic site, such as the genomic site associated with or encoding the lncRNA LOUP, described herein) using a guide polynucleotide (e.g., gRNA). Generally speaking, gRNAs come in two different systems: System 1, which uses separate crRNA and tracrRNAs that function together to guide cleavage by a Cas nuclease (e.g., Cas9), and System 2, which uses a chimeric crRNA-tracrRNA hybrid that combines the two separate guide RNAs in a single system (referred to as a single guide RNA or sgRNA: see also, e.g., Jinek et al. (2012, supra)). For System 2, gRNAs can be complementary to a target site region that is within about 100-800 base pairs (bp) upstream of a transcription start site of a gene, (e.g., within about 500 bp, about 400 bp, about 300 bp, about 200 bp, about 150 bp, about 100 bp, or about 50 bp upstream of the transcription start site), includes the transcription start site, or is within about 100-800 bp downstream of a transcription start site (e.g., within about 500 bp, about 400 bp, about 300 bp, about 200 bp, about 150 bp, about 100 bp, or about 50 bp downstream of the transcription start site). In particular embodiments, the target site region is within about 200-600 bp (e.g., 550 bp, 500 bp, 450 bp, 400 bp, 350 bp, 300 bp, 250 bp, or 200 bp) upstream of LOUP's transcription start site, and the target site region. In some embodiments, vectors (e.g., viral vectors (e.g., lentiviral vectors)) encoding more than one gRNA can be used, e.g., vectors encoding, 2, 3, 4, 5, or more gRNAs directed to different target sites or target genomic sites in the same region of the target nucleic acid molecule (e.g., a gene or other site on a chromosome). In some instances, the genomic target site and the target gene of interest are between 10-100,000 nucleotide base pairs apart (e.g., between 50-150, between 100-800 (e.g., between 125-200, between 175-300, between 275-400, between 375-500, between 475-600, between 575-700, and between 675-800), between 700-2000, between 1000-5000, between 4000-10000, between 9000-20000, between 19000-30000, between 25000-50000, between 45000-75000, or between 70000-100000).

CRISPR/Cas protein complexes can be guided to specific 17-25 nt target sites (e.g., genomic target sites) bearing an additional PAM (e.g., sequence NGG for Cas9), using a guide RNA (e.g., a single gRNA or a tracrRNA/crRNA) bearing 17-25 nts at its 5′ end that are complementary to the complementary strand of a target nucleic acid molecule (e.g., genomic DNA at a target genomic site). Thus, the gene editing system can include the use of a single guide RNA comprising a crRNA fused to a normally trans-encoded tracrRNA, e.g., a single Cas guide RNA (such as those described in Mali et al. (2013, supra)), with a sequence at the 5′ end that is complementary to the target sequence, e.g., of 17-25, optionally 20 or fewer nucleotides (nts), e.g., 20, 19, 18, or 17 nts, preferably 17 or 18 nts, of the complementary strand to a target sequence immediately 5′ of a PAM.

Existing Cas-based nucleases use gRNA-DNA heteroduplex formation to guide targeting to genomic sites of interest. However, RNA-DNA heteroduplexes can form a more promiscuous range of structures than their DNA-DNA counterparts. In effect, DNA-DNA duplexes are more sensitive to mismatches, suggesting that a DNA-guided nuclease may not bind as readily to off-target sequences, making them comparatively more specific than RNA-guided nucleases. Thus, the guide RNAs featured in the compositions and methods described herein can be hybrids, e.g., wherein one or more deoxyribonucleotides, e.g., a short DNA oligonucleotide, replaces all or part of the gRNA, e.g., all or part of the complementarity region of a gRNA. This DNA-based molecule could replace either all or part of the gRNA in a single gRNA system or alternatively might replace all of part of the crRNA and/or tracrRNA in a dual crRNA/tracrRNA system. Such a system that incorporates DNA into the complementarity region can be used to target, e.g., an intended genomic DNA site due to the general intolerance of DNA-DNA duplexes to mismatching as compared to RNA-DNA duplexes. Methods for making such duplexes are known in the art (see, e.g., Barker et al. (BMC Genomics 6: 57, 2005) and Sugimoto et al. (Biochemistry 39(37): 11270-81, 2000)).

A guide polynucleotide (e.g., a gRNA) can be any polynucleotide having a nucleic acid sequence with sufficient complementarity with the sequence of a target polynucleotide (e.g., a polynucleotide within about 800 bp (e.g., within about 500 bp, about 400 bp, about 300 bp, about 200 bp, about 150 bp, about 100 bp, or about 50 bp) upstream of the transcription start site of LOUP), a polynucleotide that includes the transcription start site of LOUP, a polynucleotide that is within about 100-800 bp (e.g., within about 500 bp, about 400 bp, about 300 bp, about 200 bp, about 150 bp, about 100 bp, or about 50 bp) downstream of a transcription start site of LOUP, or a polynucleotide within LOUP), such that the guide polynucleotide can specifically hybridize with the target polynucleotide (e.g., a polynucleotide associate with LOUP) and direct sequence-specific binding of a featured CRISPR/Cas protein complex to the target site. In some embodiments, the guide polynucleotide (e.g., gRNA) includes a sequence of ˜5-75 nucleotides that are complementary to a corresponding sequence of SEQ ID NO: 1 (e.g., SEQ ID NOs: 112-115 and 122-125). In some embodiments, the degree of complementarity between the sequence of a guide polynucleotide and corresponding sequence of the target site (e.g., a target site associated with LOUP), when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAST, Novoalign (Novocraft Technologies, ELAND (Illumina, San Diego, Calif.), SOAP (available at soap.genomics.org.cn), and Maq (available at maq.sourceforge.net). In some embodiments, a guide polynucleotide (e.g., a gRNA) has about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide polynucleotide (e.g., a gRNA) has fewer than about 75, 50, 45, 40, 35, 30, 25, 20, 15, or 12 nucleotides. The ability of a guide polynucleotide to direct sequence-specific binding of a CRISPR complex to a target site may be assessed by any suitable assay. For example, the components of a CRISPR system sufficient to form a CRISPR/Cas complex, including the guide polynucleotide to be tested, may be provided to a host cell having the corresponding target site sequence, such as by transfection with vectors encoding the components of the CRISPR/Cas complex, followed by an assessment of preferential cleavage within the sequence of the target site, such as by the incorporation of a reporter gene (e.g., a nucleic acid encoding enhanced green fluorescent protein (eGFP), or a nucleic acid encoding mCherry), or followed by an assessment of preferential gene expression, which are further described in the examples. Similarly, cleavage of a target site polynucleotide may be evaluated in a test tube by providing the target site, components of the featured CRISPR/Cas complex, including the guide polynucleotide to be tested and a control guide polynucleotide different from the test guide polynucleotide, and comparing binding or rate of cleavage at the target site between the test and control guide polynucleotide reactions. Other assay methods known to those skilled in the art can also be used.

### Delivery Methods

Vectors

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Expression vectors are well known in the art and include, but are not limited to, viral vectors and plasmids.

Vectors for use in the compositions and methods described herein contain at least one polynucleotide encoding a featured polynucleotide (e.g., a polynucleotide including a nucleic acid sequence with at least 20 nucleotides of SEQ ID NO: 1, and variants thereof with at least 85% sequence identity thereto), constructs including the lncRNA (e.g., constructs including a protein linked to a LOUP polynucleotide), a gene editing system (e.g., a CRISPR/Cas system or CRISPRa) for regulating PU.1 expression, polynucleotides encoding the gene editing system, or fragment thereof (e.g., a fragment that retains the ability to form a complex with a guide polynucleotide (e.g., a gRNA) at a target site or target genomic site), and at least one guide polynucleotide (e.g., a gRNA). The vectors may also provide additional sequence elements used for the expression of these agents and/or the integration of these polynucleotide sequences into the genome of a mammalian cell. Certain vectors that can be used for the expression of the featured polynucleotides (e.g., polynucleotides including a nucleic acid sequence with at least 20 nucleotides of SEQ ID NO: 1, and variants thereof with at least 85% sequence identity thereto), constructs including the lncRNA (e.g., constructs including a protein linked to a LOUP polynucleotide), and gene editing systems (e.g., a CRISPR/Cas system or CRISPRa) for regulating PU.1 expression include plasmids that contain regulatory sequences, such as promoter and enhancer regions, which direct transcription of the nucleic acid molecules encoding the featured components described herein. Other useful vectors for expression of the featured polynucleotides (e.g., polynucleotides including a nucleic acid sequence with at least 20 nucleotides of SEQ ID NO: 1, and variants thereof with at least 85% sequence identity thereto), constructs including the lncRNA (e.g., constructs including a protein linked to a LOUP polynucleotide), and gene editing systems (e.g., a CRISPR/Cas system or CRISPRa) for regulating PU.1 expression include polynucleotide sequences that enhance the rate of translation of these genes or improve the stability or nuclear export of the mRNA that results from gene transcription. These sequence elements include, e.g., 5′ and 3′ untranslated regions, and/or a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, and blasticidin.

In vectors encoding a featured construct, linking sequences can encode random amino acids or can contain functional sites (e.g., a cleavage site).

In some embodiments, a vector encoding a featured polynucleotide (e.g., a polynucleotide including a nucleic acid sequence with at least 20 nucleotides of SEQ ID NO: 1, and variants thereof with at least 85% sequence identity thereto), construct including the lncRNA (e.g., a construct including a protein linked to a LOUP polynucleotide, and/or gene editing system (e.g., a CRISPR/Cas system or CRISPRa) for regulating PU.1 expression can be codon optimized for expression in particular cells, such as eukaryotic cells. The eukaryotic cells may be those of, or derived from, a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis.

Accordingly, genes can be tailored for optimal gene expression in a given organism based on codon optimization. Codon usage tables are readily available, for example, at the “Codon Usage Database”, and these tables can be adapted in a number of ways. See Nakamura et al. (Nucl. Acids Res. 28:292, 2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a featured polynucleotides, constructs, CRISPR/Cas systems, and/or a gRNA, correspond to the most frequently used codon for a particular amino acid.

Viral Delivery Vehicles

Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Viral-based vectors for delivery of a desired polynucleotide and expression in a desired cell are well known in the art. Exemplary viral-based vehicles include, but are not limited to, recombinant retroviruses (e.g., a lentiviral vector, see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127), adenovirus vectors, alphavirus-based vectors (e.g., Sindbis virus vectors, Semliki forest virus), Ross River virus, adeno-associated virus (AAV) vectors (see, e.g., PCT Publication Nos. WO 94/12649, WO 93/03769; WO 93/19191; WO 94/28938; WO 95/11984 and WO 95/00655), vaccinia virus (e.g., Modified Vaccinia virus Ankara (MVA) or fowlpox), Baculovirus recombinant system, and herpes virus. Further examples of viral vectors for delivery of the featured polynucleotides (e.g., a polynucleotide including a nucleic acid sequence with at least 20 (or all) nucleotides of the lncRNA LOUP (SEQ ID NO: 1), and variants thereof with at least 85% sequence identity thereto), constructs including the polynucleotide (e.g., constructs including a protein linked to a LOUP polynucleotide), and/or gene editing systems (e.g., a CRISPR/Cas system or CRISPRa) for regulating PU.1 expression include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double-stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the entire contents of which is hereby incorporated by reference.

Exemplary viral vectors include lentiviral vectors, AAVs, and retroviral vectors. Lentiviral vectors and AAVs can integrate into the genome without cell divisions, and both types have been tested in pre-clinical animal studies.

Methods for preparation of AAVs are described in the art, e.g., in U.S. Pat. Nos. 5,677,158, 6,309,634, and 6,683,058, the entire contents of each of which is incorporated herein by reference.

Methods for preparation and in vivo administration of lentiviruses are described in US 20020037281, the entire contents of which is hereby incorporated by reference. Lentiviral vectors (LVs) transduce a wide range of dividing and non-dividing cell types with high efficiency, conferring stable, long-term expression of the transgene. An overview of optimization strategies for packaging and transducing LVs is provided in Delenda (J. Gen Med 6: S125, 2004), the entire contents of which are incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and Woodchuck Hepatitis Virus (WHP) Posttranscriptional Regulatory Element (WPRE) sequence. The vector may also include an IRES sequence that permits the expression of multiple polypeptides from a single promoter.

The vector used in the methods and compositions described herein may include multiple promoters that permit expression of more than one polynucleotide and/or polypeptide. The vector used in the methods and compositions described herein may include a protein cleavage site that allows expression of more than one polypeptide. Examples of protein cleavage sites that allow expression of more than one polypeptide are described in, e.g., Klump et al. (Gene Ther 8:811 2001), Osborn et al. (Molecular Therapy 12:569, 2005), Szymczak and Vignali (Expert Opin Biol Ther. 5:627, 2005), and Szymczak et al. (Nat Biotechnol. 22:589, 2004), the disclosures of which are incorporated herein by reference. It will be readily apparent to one skilled in the art that other elements that permit expression of multiple polypeptides identified in the future are useful and may be utilized in the vectors suitable for use with the compositions and methods described herein.

The vector used in the methods and compositions described herein may be a clinical grade vector.

The viral vector may also include viral regulatory elements, which are components of delivery vehicles used to introduce nucleic acid molecules into a host cell. The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions. One skilled in the art would also appreciate that as other viral regulatory elements are identified, these may be used with the viral vectors described herein.

Non-Viral Delivery Vehicles

Several non-viral vehicles can be used for delivery of the featured polynucleotides (e.g., a polynucleotide having a nucleic acid sequence with at least 20 (or all) nucleotides of the lncRNA LOUP (SEQ ID NO: 1), and variants thereof with at least 85% (e.g., at least 86%, at least 87%, at least 88%, at least 89%, at least 90%, at least 91%, at least 92%, at least 93%, at least 94%, at least 95%, at least 96%, at least 97%, at least 98%, or at least 99%), sequence identity thereto), constructs including the lncRNA (e.g., a construct including a protein linked to a LOUP polynucleotide), and a gene editing system (e.g., a CRISPR/Cas system or CRISPRa) for regulating PU.1 expression. These non-viral vectors include, e.g., prokaryotic and eukaryotic vectors (e.g., yeast- and bacteria-based plasmids), as well as plasmids for expression in mammalian cells. Methods of introducing the vectors into a host cell and isolating and purifying the expressed protein are also well known in the art (e.g., Molecular Cloning: A Laboratory Manual, second edition, Sambrook, et al. 1989, Cold Spring Harbor Press). Examples of host cells include, but are not limited to, mammalian cells, such as NS0, CHO cells, HEK and COS, and bacterial cells, such as E. coli.

Other non-viral delivery vehicles include polymeric, biodegradable microparticle, or microcapsule delivery devices known in the art. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules.

The composition of the liposome is usually a combination of phospholipids, usually in combination with steroids, in particular cholesterol. Other phospholipids or other lipids may also be used. The physical characteristics of liposomes depend on pH, ionic strength, and the presence of divalent cations.

Lipids useful in liposome production include phosphatidyl compounds, such as phosphatidylglycerol, phosphatidylcholine, phosphatidylserine, phosphatidyl-ethanolamine, sphingolipids, cerebrosides, and gangliosides. Exemplary phospholipids include egg phosphatidylcholine, dipalmitoylphosphatidylcholine, and distearoyl-phosphatidylcholine. The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255.

Pharmaceutical Compositions

The disclosure also includes pharmaceutical compositions containing a polynucleotide described herein (e.g., all or at least about 20 or more nucleotides of the long non-coding RNA, LOUP (SEQ ID NO: 1), and variants thereof with at least 85% or more sequence identity thereto, a polynucleotide encoding the lncRNA (e.g., a polynucleotide encoding at least 20 nucleotides of SEQ ID NO: 1), a vector (e.g., a viral vector) including the lncRNA or a polynucleotide encoding the lncRNA, a construct including the lncRNA (e.g., a construct including a protein linked to a LOUP polynucleotide), a gene editing system (e.g., a CRISPR/Cas system or CRISPRa) for regulating PU.1 expression, a polynucleotide encoding the gene editing system, and a vector (e.g., a viral vector) including polynucleotides encoding the gene editing system, as described herein. The pharmaceutical composition can be prepared as a composition containing a pharmaceutically acceptable carrier, excipient, or stabilizer known in the art (Remington: The Science and Practice of Pharmacy 20th Ed., 2000, Lippincott Williams and Wilkins, Ed. K. E. Hoover). The compositions may also be provided in the form of a lyophilized formulation, as an aqueous solution, or as a pharmaceutical product suitable for direct administration.

Acceptable carriers, excipients, or stabilizers that can be used to prepare a pharmaceutical composition are considered to be non-toxic to a recipient, e.g., when included in the composition at therapeutic dosages and concentrations, and may include buffers such as phosphate, citrate, and other organic acids; antioxidants including ascorbic acid and methionine; preservatives (e.g., octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, marmose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Pharmaceutically acceptable excipients are further described herein.

The compositions (e.g., when used in the methods described herein) generally include, by way of example and not limitation, an effective amount (e.g., an amount sufficient to mitigate disease, alleviate a symptom of disease and/or prevent or reduce the progression of disease) of a long non-coding RNA (e.g., a LOUP RNA), a polynucleotide encoding the lncRNA (e.g., a polynucleotide having at least 20 nucleotides of SEQ ID NO: 1), a vector (e.g., a viral vector) including a polynucleotide encoding the lncRNA, a construct including the lncRNA (e.g., a construct including a protein linked to a LOUP polynucleotide), a gene editing system (e.g., a CRISPR/Cas system or CRISPRa) for regulating PU.1 expression, a polynucleotide encoding the gene editing system, and/or a vector (e.g., a viral vector) including polynucleotides encoding the gene editing system, as described herein.

The composition may be formulated to include between about 1 μg/mL and about 1 g/mL of the long non-coding RNA (e.g., LOUP RNA), the polynucleotide encoding the lncRNA (e.g., a polynucleotide having at least 20 nucleotides of SEQ ID NO: 1), the vector (e.g., a viral vector) including the polynucleotide encoding the lncRNA, the construct including the lncRNA (e.g., a construct including a protein linked to a LOUP polynucleotide), the gene editing system (e.g., a CRISPR/Cas system or CRISPRa) for regulating PU.1 expression, the polynucleotide encoding the gene editing systems, and/or the vector (e.g., a viral vector) including the polynucleotide(s) encoding the gene editing system, or any combination thereof (e.g., between 10 μg/mL and 300 μg/mL, 20 μg/mL and 120 μg/mL, 40 μg/mL and 200 μg/mL, 30 μg/mL and 150 μg/mL, 40 μg/mL and 100 μg/mL, 50 μg/mL and 80 μg/mL, or 60 μg/mL and 70 μg/mL, or 10 mg/mL and 300 mg/mL, 20 mg/mL and 120 mg/mL, 40 mg/mL and 200 mg/mL, 30 mg/mL and 150 mg/mL, 40 mg/mL and 100 mg/mL, 50 mg/mL and 80 mg/mL, 60 mg/mL and 70 mg/mL, or 100 mg/ml and 1 g/ml (e.g., 150 mg/ml, 200 mg/ml, 250 mg/ml, 300 mg/ml, 350 mg/ml, 400 mg/ml, 450 mg/ml, 500 mg/ml, 550 mg/ml, 600 mg/ml, 650 mg/ml, 700 mg/ml, 750 mg/ml, 800 mg/ml, 850 mg/ml, 900 mg/ml, or 950 mg/ml).

A composition containing a non-viral vector of the disclosure may contain a unit dose containing a quantity of long non-coding RNA (e.g., LOUP RNA), polynucleotides encoding the lncRNA (e.g., a polynucleotide having at least 20 nucleotides of SEQ ID NO: 1), vectors (e.g., viral vectors) including polynucleotides encoding the lncRNA, constructs including the lncRNA (e.g., constructs including a protein linked to a LOUP polynucleotide), gene editing system (e.g., a CRISPR/Cas system or CRISPRa) for regulating PU.1 expression, polynucleotides encoding the gene editing systems, and vectors (e.g., viral vectors) including polynucleotides encoding the gene editing system from 10 μg to 10 mg (e.g., from 25 μg to 5.0 mg, from 50 μg to 2.0 mg, or from 100 μg to 1.0 mg of polynucleotides, e.g., from 10 μg to 20 μg, from 20 μg to 30 μg, from 30 μg to 40 μg, from 40 μg to 50 μg, from 50 μg to 75 μg, from 75 μg to 100 μg, from 100 μg to 200 μg, from 200 μg to 300 μg, from 300 μg to 400 μg, from 400 μg to 500 μg, from 500 μg to 1.0 mg, from 1.0 mg to 5.0 mg, or from 5.0 mg to 10 mg of polynucleotides, e.g., about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 600 μg, about 700 μg, about 750 μg, about 1.0 mg, about 2.0 mg, about 2.5 mg, about 5.0 mg, about 7.5 mg, or about 10 mg of polynucleotides). The long non-coding RNA (e.g., LOUP RNA), polynucleotides encoding the lncRNA (e.g., a polynucleotide having at least 20 nucleotides of SEQ ID NO: 1), vectors (e.g., viral vectors) including polynucleotides encoding the lncRNA, constructs including the lncRNA (e.g., constructs including a protein linked to a LOUP polynucleotide), gene editing system (e.g., a CRISPR/Cas system or CRISPRa) for regulating PU.1 expression, polynucleotides encoding the gene editing systems, and vectors (e.g., viral vectors) including polynucleotides encoding the gene editing system may be formulated in the unit dose above in a volume of 0.1 ml to 10 ml (e.g., 0.2 ml, 0.5 ml, 0.75 ml, 1 ml, 1.5 ml, 2 ml, 3 ml, 4 ml, 5 ml, 6 ml, 7 ml, 8 ml, 9 ml, or 10 ml).

The compositions may also include a viral vector containing a nucleic acid sequence encoding a featured polynucleotide (e.g., a polynucleotide including at least 20 nucleotides of SEQ ID NO: 1), constructs including the lncRNA (e.g., constructs including a protein linked to a LOUP polynucleotide), and gene editing system (e.g., a CRISPR/Cas system or CRISPRa) for regulating PU.1 expression, polynucleotides encoding the gene editing systems or a composition containing a featured polynucleotide (e.g., a polynucleotide including at least 20 nucleotides of SEQ ID NO: 1), constructs including the lncRNA (e.g., constructs including a protein linked to a LOUP polynucleotide), and gene editing system (e.g., a CRISPR/Cas system or CRISPRa) for regulating PU.1 expression, polynucleotides encoding the gene editing systems. The compositions containing viral particles can be prepared in 1 ml to 10 ml (e.g., 1 ml, 2 ml, 3 ml, 4 ml, 5 ml, 6 ml, 7 ml, 8 ml, 9 ml, or 10 ml) aliquots, having a viral titer of at least about 1×10⁶ pfu/ml (plaque-forming unit/milliliter), and, in general, not exceeding 1×10¹¹ pfu/ml. Thus, the composition may contain, for example, about 1×10⁶ pfu/ml, about 2×10⁶ pfu/ml, about 4×10⁶ pfu/ml, about 1×10⁷ pfu/ml, about 2×10⁷ pfu/ml, about 4×10⁷ pfu/ml, about 1×10⁸ pfu/ml, about 2×10⁸ pfu/ml, about 4×10⁸ pfu/ml, about 1×10⁹ pfu/ml, about 2×10⁹ pfu/ml, about 4×10⁹ pfu/ml, about 1×10¹⁰ pfu/ml, about 2×10¹⁰ pfu/ml, about 4×10¹⁰ pfu/ml, and about 1×10¹¹ pfu/ml. The composition can also contain a pharmaceutically acceptable carrier described herein. The pharmaceutically acceptable carrier can be, for example, a liquid carrier such as a saline solution, protamine sulfate (Elkins-Sinn, Inc., Cherry Hill, N.J.) or Polybrene (Sigma) as well as others described herein.

Methods for Diagnosing a Subject as a LOUP-Related Disease or Disorder

Also provided herein are methods of diagnosing a disease or disorder (e.g., a cancer (e.g., AML, liver cancer, or myeloma), Alzheimer's disease, or asthma) in a subject (e.g., a subject suspected of having a disease or disorder). The diagnostic method can be performed by determining a level of the transcription factor PU.1 in a subject or a level of LOUP expression in a subject.

For example, a sample (e.g., a tissue sample, a blood sample, a cell sample, or a fluidic sample) can be obtained from a subject (e.g., a subject suspected of having a disease or disorder) and analyzed for PU.1 expression. The level of PU.1 expression can be compared to a standard or reference level (e.g., a control sample, in which a known expression level of PU.1 has been linked to the presence or absence of the disease or disorder) or to a sample from a reference subject (e.g., a subject known to be healthy (e.g., to lack the disease or disorder) or a subject known to have the disease or disorder). Comparison of the PU.1 level to the standard or reference level can confirm the presence or absence of the disease or disorder in the subject being tested.

For example, a subject determined to have decreased expression of PU.1, as compared to a standard or reference, can be identified as having or at risk of developing a cancer (e.g., AML, liver cancer, or myeloma). Alternatively, a subject determined to have increased expression of PU.1, as compared to a standard or reference, can be identified as having or at risk of developing Alzheimer's disease or asthma.

For example, a sample (e.g., a tissue sample, a blood sample, a cell sample, or a fluidic sample) can be obtained from a subject (e.g., a subject suspected of having a disease or disorder) and analyzed for LOUP expression. The level of LOUP expression can be compared to a standard or reference level (e.g., a control sample, in which a known expression level of LOUP has been linked to the presence or absence of the disease or disorder) or to a sample from a reference subject (e.g., a subject known to be healthy (e.g., to lack the disease or disorder) or a subject known to have the disease or disorder). Comparison of the LOUP level to the standard or reference level can confirm the presence or absence of the disease or disorder in the subject being tested.

For example, a subject determined to have decreased expression of LOUP, as compared to a standard or reference, can be identified as having or at risk of developing a cancer (e.g., AML, liver cancer, or myeloma). Alternatively, a subject determined to have increased expression of LOUP, as compared to a standard or reference, can be identified as having or at risk of developing Alzheimer's disease or asthma.

Also provided are methods of diagnosing a subject as having a cancer (e.g., AML) that is susceptible to differentiation therapy with all-trans retinoic acid (ATRA) based on LOUP expression. A sample (e.g., a tissue sample, a blood sample, a cell sample, or a fluidic sample) from a subject (e.g., a subject having or suspected of having a cancer (e.g., AML)) can be analyzed for LOUP expression and compared to a standard or reference level (e.g., a control sample, in which a known expression level of LOUP has been linked to the presence or absence of the disease or disorder) or to a sample from a reference subject (e.g., a subject known to be healthy (e.g., to lack the disease or disorder) or a subject known to have the disease or disorder). Comparison of the LOUP level to the standard or reference level can be used to determine if the subject is likely to be sensitive to differentiation therapy with ATRA. For example, low levels of LOUP (relative to a standard or reference) would indicate resistance of the cancer to ATRA therapy.

Gene sequencing methods (e.g., next-generation gene sequencing methods, e.g., high-throughput sequencing, including but not limited to, Illumina sequencing, Roche 454 sequencing, Ion torrent: Proton/PGM sequencing, and SOLiD sequencing) can be used to analyze PU.1 and/or LOUP expression for the diagnosis of a disease or disorder.

Methods of Treatment

A subject in need of treatment for a disease or disorder associated with reduced expression of the transcription factor PU.1 (e.g., a cancer, such as AML, liver cancer, or myeloma) can be administered a composition described herein that increases expression of PU.1. Alternatively, a subject in need of treatment for a disease or disorder associated with increased expression of the transcription factor PU.1 (e.g., Alzheimer's disease or asthma) can be administered a composition described herein that decreases expression of PU.1. Each of these methods are described below.

For treatment of a disease or disorder associated with reduced expression of PU.1, generally, a composition containing the featured polynucleotide (e.g., a polynucleotide including at least 20 nucleotides of SEQ ID NO: 1) can be administered (e.g., intravenously) to a subject (e.g., a subject in need thereof, such as a human) as a medicament (e.g., for treating a medical condition (e.g., a cancer (e.g., a PU.1 associated cancer (e.g., AML, liver cancer, or myeloma)))). The featured polynucleotide described herein can be used to induce the expression of tumor suppressor gene PU.1, thereby treating the disease or disorder. In some embodiments, the featured polynucleotide can be delivered as a vector (e.g., a viral vector or non-viral vector) described herein. In certain embodiments, the featured polynucleotide can be delivered as a vector including a nucleic acid encoding the featured polynucleotide (e.g., a polynucleotide including at least 20 nucleotides of SEQ ID NO: 1) as described herein. In some embodiments, the vector is a viral vector (e.g., a lentiviral vector or an AAV vector). Gene sequencing methods (e.g., next-generation gene sequencing methods, e.g., high-throughput sequencing, including but not limited to, Illumina sequencing, Roche 454 sequencing, Ion torrent: Proton/PGM sequencing, and SOLiD sequencing) can be used to identify a subject in need thereof (e.g., a subject with a PU.1 associated cancer (e.g., AML, liver cancer, or myeloma)).

Alternatively, or in addition, a composition containing the featured gene editing system can be administered (e.g., intravenously) to a subject (e.g., a subject in need thereof, such as a human) as a medicament (e.g., for treating a medical condition (e.g., a PU.1 associated medical condition (e.g., a PU.1 associated cancer (e.g., AML, liver cancer, or myeloma)), or asthma)). In some embodiments, a composition including the featured gene editing system can be administered (e.g., intravenously or intracranially) to a subject (e.g., a subject in need thereof, such as a human) as a medicament (e.g., for treating a medical condition (e.g., a PU.1 associated medical condition (e.g., Alzheimer's Disease). In some embodiments, a composition including the featured gene editing system can be administered to a subject (e.g., a subject in need thereof, such as a human) as a medicament (e.g., for treating a medical condition (e.g., a PU.1 associated medical condition (e.g., a PU.1 associated cancer (e.g., AML, liver cancer, or myeloma)), Alzheimer's Disease, or asthma)) by any method that allows the featured gene editing system to target a genomic site associated with PU.1 expression. The gene editing system described herein can be used to efficiently target any of a number of genomic sites associated with a medical condition (e.g., a PU.1 associated medical condition). Gene sequencing methods (e.g., next-generation gene sequencing methods, e.g., high-throughput sequencing, including but not limited to, Illumina sequencing, Roche 454 sequencing, Ion torrent: Proton/PGM sequencing, and SOLiD sequencing) can be used to identify PU.1 or LOUP expression, which can identify the subject as one in need of treatment. The gene sequencing data can also be used to identify a suitable target site(s) or target genomic site(s) to be targeted by a guide polynucleotide(s) (e.g., a guide RNA(s) directed to a target site associated with LOUP) so as to limit any effect at off target sites. Target sites and target genomic sites will, preferably, but not necessarily, be uniquely associated with LOUP (e.g., a unique target site directing the CRISPR/Cas system to LOUP as described herein), and to the Cas nuclease of the featured CRISPR/Cas system.

The featured long non-coding RNA (e.g., LOUP RNA), polynucleotides encoding the lncRNA (e.g., a polynucleotide having at least 20 nucleotides of SEQ ID NO: 1), vectors (e.g., viral vectors) including polynucleotides encoding the lncRNA, constructs including the lncRNA (e.g., constructs including a protein linked to a LOUP polynucleotide), gene editing system (e.g., a CRISPR/Cas system or CRISPRa) for regulating PU.1 expression, polynucleotides encoding the gene editing systems, and vectors (e.g., viral vectors) including polynucleotides encoding the gene editing system can be administered to a subject in need thereof (e.g., a human) to alter (e.g., increase or decrease) the expression of tumor associated gene PU.1. Compositions and methods for delivering the featured polynucleotides (e.g., a polynucleotide having at least 20 nucleotides of SEQ ID NO: 1) and/or CRISPR/Cas system or CRISPRa components include, e.g., a vector (e.g., a viral vector, such as a lentiviral vector particle), and non-vector delivery vehicles (e.g., nanoparticles), as discussed above. For example, the featured polynucleotides and CRISPR/Cas system described herein may be formulated for and/or administered to a subject in need thereof (e.g., a subject who has been diagnosed with a medical condition associated with anti-tumor proliferating gene PU.1 (e.g., a cancer (e.g., AML, liver cancer, or myeloma), Alzheimer's disease, or asthma)) by a variety of routes, such as local administration at or near the site affected by the medical condition (e.g., injection near a cancer, direct administration to the central nervous system (CNS) (e.g., intracranial, intracerebral, intraventricular, intrathecal, intracisternal, or stereotactic administration) for treating a neurological medical condition, such as Alzheimer's disease), intravenous, parenteral, intradermal, transdermal, intramuscular, intranasal, subcutaneous, percutaneous, intratracheal, intraperitoneal, intraarterial, intravascular, inhalation, perfusion, lavage, topical, and oral administration. The most suitable route for administration in any given case may depend on the particular subject, pharmaceutical formulation methods, administration methods (e.g., administration time and administration route), the subject's age, body weight, sex, severity of the disease being treated, the subject's diet, and the subject's excretion rate. Compositions may be administered once, or more than once (e.g., once annually, twice annually, three times annually, bi-monthly, monthly). For local administration, the featured polynucleotides (e.g., polynucleotides encoding the lncRNA (e.g., a polynucleotide having at least 20 nucleotides of SEQ ID NO: 1), constructs including a LOUP polynucleotide, gene editing system (e.g., CRISPR/Cas system or CRISPRa), and featured viral vectors containing nucleic acid sequences encoding the featured polynucleotides, constructs, or gene editing system may be administered by any means that places the polynucleotides, constructs, or gene editing system in a desired location, including catheter, syringe, shunt, stent, or microcatheter, pump. The subject can be monitored for PU.1 expression after treatment. Methods of monitoring the expression of PU.1 are discussed further below. The dosing regimen may be adjusted based on the monitoring results to ensure a therapeutic response.

Generally, the methods can include administering a composition containing the polynucleotide (e.g., a polynucleotide including at least 20 nucleotides of SEQ ID NO: 1), a construct including a LOUP polynucleotide, or the gene editing system (e.g., a CRISPR/Cas system), either incorporated as a nucleic acid molecule (e.g., in a vector, such as a viral vector) encoding the polynucleotide, construct, or the components of the gene editing system (e.g., Cas protein and guide polynucleotides (e.g., guide RNA)) to a subject in need thereof. Alternatively, the methods can include administering the gene editing system in protein form (e.g., as a composition containing a Cas protein in combination with one or more guide polynucleotide(s) (e.g., gRNA(s))). The compositions can be administered (e.g., intravenously or intracranially) to a subject (e.g., a subject in need thereof) as a medicament for the treatment of a medical condition associated with PU.1 expression.

### Dosage and Administration

The pharmaceutical compositions described herein can be administered to a subject (e.g., a human) in a variety of ways. For example, the pharmaceutical compositions may be formulated for and/or administered orally, buccally, sublingually, parenterally, intravenously, subcutaneously, intramedullary, intranasally, as a suppository, using a flash formulation, topically, intradermally, subcutaneously, via pulmonary delivery, via intra-arterial injection, ophthalmically, optically, intrathecally, or via a mucosal route.

A viral vector, such as a lentiviral vector, can be administered in an amount effective to produce a therapeutic effect in a subject. The exact dosage of viral particles to be administered is dependent on a variety of factors, including the age, weight, and sex of the subject to be treated, and the nature and extent of the disease or disorder to be treated. The viral particles can be administered as part of a preparation having a titer of viral vectors of at least 1×10⁶ pfu/ml (plaque-forming unit/milliliter), and in general not exceeding 1×10¹¹ pfu/ml, in a volume between about 0.5 ml to about 10 ml (e.g., 1 ml, about 2 ml, about 3 ml, about 4 ml, about 5 ml, about 6 ml, about 7 ml, about 8 ml, about 9 ml, or about 10 ml). Thus, the administered composition may contain, for example, about 1×10⁶ pfu/ml, about 2×10⁶ pfu/ml, about 4×10⁶ pfu/ml, about 1×10⁷ pfu/ml, about 2×10⁷ pfu/ml, about 4×10⁷ pfu/ml, about 1×10⁸ pfu/ml, about 2×10⁸ pfu/ml, about 4×10⁸ pfu/ml, about 1×10⁹ pfu/ml, about 2×10⁹ pfu/ml, about 4×10⁹ pfu/ml, about 1×10¹⁰ pfu/ml, about 2×10¹⁰ pfu/ml, about 4×10¹⁰ pfu/ml, and about 1×10¹¹ pfu/ml. The dosage may be adjusted to balance the therapeutic benefit against any side effects.

Any of the non-viral vectors of the present invention can be administered to a subject in a dosage from about 10 μg to about 10 mg of polynucleotides (e.g., from 25 μg to 5.0 mg, from 50 μg to 2.0 mg, or from 100 μg to 1.0 mg of polynucleotides, e.g., from 10 μg to 20 μg, from 20 μg to 30 μg, from 30 μg to 40 μg, from 40 μg to 50 μg, from 50 μg to 75 μg, from 75 μg to 100 μg, from 100 μg to 200 μg, from 200 μg to 300 μg, from 300 μg to 400 μg, from 400 μg to 500 μg, from 500 μg to 1.0 mg, from 1.0 mg to 5.0 mg, or from 5.0 mg to 10 mg of polynucleotides, e.g., about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 600 μg, about 700 μg, about 750 μg, about 1.0 mg, about 2.0 mg, about 2.5 mg, about 5.0 mg, about 7.5 mg, or about 10 mg of polynucleotides) in a volume of a pharmaceutically acceptable carrier between about 0.1 ml to about 10 ml (e.g., about 0.2 ml, about 0.5 ml, about 1 ml, about 1.5 ml, about 2 ml, about 3 ml, about 4 ml, about 5 ml, about 6 ml, about 7 ml, about 8 ml, about 9 ml, or about 10 ml).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, e.g., a pH in the physiologically acceptable range. Examples of buffer solutions include saline, phosphate buffered saline, Tris buffered saline, Hank's buffered saline, and the like.

In some embodiments, the method may also include a step of assessing the subject for successful alteration in PU.1 expression (e.g., an increase or decrease in PU.1 expression). In some embodiments, the subject in need of a treatment (e.g., a human subject having a disease or disorder associated with PU.1 expression) is monitored for alleviation of the symptoms of the disease or disorder (e.g., cancer (e.g., AML, liver cancer, or myeloma), Alzheimer's disease, or asthma). In these instances, the subject will be monitored for a reduction or decrease in the side effects of a disease or disorder, such as those described herein, or the risk or progression of the disease or disorder, may be relative to a subject who did not receive treatment, e.g., a control, a baseline, or a known control level or measurement. The reduction or decrease may be, e.g., by about 1%, 2%, 3%, 4%, 5%, 6%, 7%, 8%, 9%, 10%, 20%, 30%, 40%, 50%, 60%, 70%, 80%, 90%, 95%, 97%, 99%, or about 100% relative to a subject who did not receive treatment or a control, baseline, or known control level or measurement, or may be a reduction in the number of days during which the subject experiences the disease or disorder or associated symptoms (e.g., a reduction of 1-30 days, 2-12 months, 2-5 years, or 6-12 years). The results of monitoring a subject's response to a treatment can be used to adjust the treatment regimen.

In certain embodiments, the gene editing system can be used to introduce a genetic mutation (e.g., a missense mutation, a nonsense mutation, an insertion, a deletion, a duplication, a frameshift mutation, or a repeat expansion) or a gene of interest (e.g., a LOUP gene) into a genome of a target cell. In these instances, the mutation may be inserted to treat (e.g., in a human) a disease or disorder (e.g., Alzheimer's Disease or asthma) in a subject in need thereof. In these instances, the subject (e.g., a human subject) can be monitored for a change in the disease or disorder (e.g., a change in the progression of the disease or disorder or in a lessening of etiologies of the disease or disorder in a subject that has been treated, or, alternatively, in the production or increase in the etiologies of a disease or disorder in a subject (e.g., a research animal) that has had one or more cells edited to replicate the disease or disorder). The changes can be monitored relative to a subject who did not receive the treatment or editing modification, e.g., a control, a baseline, or a known control level or measurement. The change may be, e.g., by about 1%, 2%, 3%, 4%, 5%, 6%, 7%, 8%, 9%, 10%, 20%, 30%, 40%, 50%, 60%, 70%, 80%, 90%, 95%, 97%, 99%, or about 100% relative to a subject who did not receive treatment or editing modification or a control, baseline, or known control level or measurement, or may be a change in the number of days during which the subject experiences the disease or disorder or associated symptoms (e.g., a reduction of 1-30 days, 2-12 months, 2-5 years, or 6-12 years in a treated subject).

In certain embodiments, the treatment is monitored at the protein level. Successful expression of the featured gene editing system in a cell or tissue can be assessed by standard immunological assays, for example the ELISA (see, Ausubel et al. Current Protocols in Molecular Biology, Greene Publishing Associates, New York, V. 1-3, 2000; Harlow and Lane, Antibodies, A Laboratory Manual, Cold Spring Harbor Laboratory, 1988, the entire contents of which is hereby incorporated by reference).

Alternatively, the biological activity of LOUP and/or PU.1 can be measured directly by the appropriate assay, for example, the assays provided herein. The skilled artisan would be able to select and successfully carry out the appropriate assay to assess the biological activity of the gene product of interest in a particular sample. Such assays (e.g., real time PCR (qPCR)) might require removing a sample (e.g., cells or tissue) from the subject to use in the assay. Expression of the featured polynucleotides (e.g., polynucleotides encoding the lncRNA (e.g., a polynucleotide having at least 20 nucleotides of SEQ ID NO: 1)) or successful gene editing using a gene editing system (e.g., CRISPR/Cas system) for delivering the same, may be monitored by any of a variety of detection methods available in the art, such as those described herein. For example, gene sequencing methods can be used to identify the successful insertion of the polynucleotide encoding the features polynucleotides using the gene editing system described herein. The subsequent expression of the target gene molecule (e.g., LOUPor PU.1) can be monitored.

### Kits

Also featured are kits containing any one or more of the polynucleotides (e.g., polynucleotides including at least 20 nucleotides of SEQ ID NO: 1), constructs including, e.g., a protein and a polynucleotide (e.g., a LOUP polynucleotide), CRISPR/Cas system elements, or vectors comprising one or more of the polynucleotides, constructs, or CRISPR/Cas system elements disclosed in the above methods and compositions. Kits of the invention include one or more containers comprising, for example, one or more of a featured polynucleotide (e.g., polynucleotides including at least 20 nucleotides of SEQ ID NO: 1), or fragment thereof, construct including the lncRNA (e.g., a construct including a protein linked to a LOUP polynucleotide), CRISPR/Cas system or component thereof, one or more guide polynucleotide(s) (e.g., gRNAs), and/or one or more containers with nucleic acids encoding one or more of the polynucleotides, constructs, or CRISPR/Cas systems or components thereof, such as, e.g., a vector containing the nucleic acid molecules (e.g., a viral vector, such as a lentiviral vector, an adenoviral vector, or an AAV vector), and, optionally, instructions for use in accordance with any of the methods described herein.

Generally, these instructions comprise a description of administration or instructions for performance of an assay (e.g., a LOUP or PU.1 expression assay). The containers may be unit doses, bulk packages (e.g., multi-dose packages), or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also envisioned.

The kits may be provided in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (e.g., the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). Kits may optionally provide additional components such as buffers and interpretive information. Normally, the kit comprises a container and a label or package insert(s) on or associated with the container.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

The following examples discuss identification and uses of long non-coding RNA (e.g., LOUP RNA) and polynucleotides encoding the same. Also described are vectors (e.g., viral vectors) including polynucleotides encoding the lncRNA and use of a gene editing system (e.g., a CRISPR/Cas system) to regulate PU.1 expression. Finally, examples are provided showing methods of diagnosing, treating, or preventing a disease (e.g., cancer (e.g., PU.1 associated cancer (e.g., AML, liver cancer, and myeloma)), Alzheimer's Disease, or asthma) associated with LOUP and/or PU.1 expression, as well as methods of diagnosing treatment (e.g., ATRA) responsiveness in a subject with cancer (e.g., AML, liver disease, or myeloma).

### Example 1. Experimental Model and Subject Details

Long-range enhancer-promoter interactions result in dynamic expression patterns of lineage genes. How these communications occur in specific cell types and at specific gene loci remain elusive. Here we investigate whether RNAs coordinate with transcription factors to drive lineage gene transcription. In an integrated genome-wide approach surveying for gene loci exhibiting concurrent RNA- and DNA-interactions with RUNX1 protein (described below), we identified a long noncoding RNA (lncRNA) arising from the upstream region of the myeloid master regulator PU.1. This myeloid-specific and polyadenylated lncRNA acts as a transcriptional inducer of PU.1 by modulating the formation of an active chromatin loop at the PU.1 locus. The lncRNA utilizes embedded transposable element variants to bind and recruit RUNX1 to both the enhancer and the promoter, resulting in the formation of the enhancer-promoter complex. These findings provide mechanistic insight, highlighting the important role of the interplay between cell type-specific RNAs and transcription factors in lineage-gene activation.

Cell Lines and Cell Culture

U937, HL-60, K562, HEK293T, RAW 264.7, NB4, Jurkat, Kasumi-1 and THP-1 cells were obtained from American Type Culture Collection (ATCC). U937, HL-60, NB4, Jurkat, Kasumi-1 and K562 cells were cultured in RPMI-1640 supplemented with 10% (vol/vol) fetal bovine serum (FBS; Cellgro) and 1% penicillin-streptomycin. THP-1 cells were cultured in the same medium supplemented with 2-mercaptoethanol to a final concentration of 0.05 mM. HEK293T and RAW 264.7 cells were cultured in DMEM supplemented with 10% (vol/vol) FBS and 1% penicillin-streptomycin. All cells were grown at 37° C. in 5% (vol/vol) CO2 and humidified incubators.

Lentiviral Generation

Lentiviral particles were generated following our optimized protocol (Trinh et al., J. Cell. Sci. 128: 3055-3067, 2015). Briefly, HEK293T cells were plated overnight to reach 80-85% confluency on the next day. Cells were then co-transfected with viral expression vector plus packaging plasmids (pMD2.G and psPAX2, Addgene) using Lipofectamine 2000 (Life Technologies). At 48 h and 72 h thereafter, culture supernatants were collected and filtered through a 0.45-mm PVDF filter (Millipore). Viruses were further concentrated using PEG-it® Virus Precipitation Solution (System Biosciences).

Plasmid Generation

LOUP cDNA in pCMV-SPORT6 plasmid (Dharmacon) was sub-cloned into the lentiviral pCDH-MSCV-MCS-EF1-copGFP expression vector that carries copGFP marker (System Biosciences).

Generation of CRISPR Knockout Cells (CRISPRko)

FUCas9Cherry (Aubrey et al., Cell Rep. 10: 1422-1432, 2015) (Addgene) was used as expression vector to generate mCherry-Cas9 lentiviral particles as described above. U937 cells were transduced with these particles using TRANSDUX® reagent (System Biosciences). Cas9-stable cells were then selected by several rounds of FACS sorting for mCherry positivity. LOUP-targeting sgRNAs were designed using Cas-Designer (Park et al., Bioinformatics 31:4014-4016, 2015) and cloned into pLVx U6se EF1a sfPac vector which carry eGFP. To avoid disruption of the URE, known to be critical for PU.S1 induction (Li et al. Blood 98: 2958-2965, 2001), single-guide RNAs (sgRNA) targeting two distinct regions of the LOUPgene: (1) the LOUP intronic area downstream of the URE, and (2) the intronic area right upstream of the second exon of the LOUP gene (˜15 kb downstream from the URE) were designed. Cas9-stable cells were then transduced with eGFP-sgRNA lentiviruses. Cells expressing high levels of both eGFP and mCherry were FACS sorted, one cell per well, into 96-well plates. Genomic DNA from cell clones were isolated using DNeasy Blood & Tissue Kit kit (QIAGEN) and used for P2R amplifying CRISPR/Cas9 target sites. PfR products were sequenced and indel profile were analyzed by ICE software (Hsiau, et al. BioRxiv 251 082 2018). Cell clones having homozygous indels were verified by Sanger sequencing. Primer and sgRNA sequences are provided in Table 3.

TABLE 3
Primer and sgRNA sequences

| | | | | SEQ ID |
|---|---|---|---|---|
| Function | Oligo name | Description | Sequence 5′ to 3′ | NO: |
| LOUP RT-PCR | hLOUP F | forward primer for | GGCTTCAGCCTCCCT | SEQ ID |
| to check exon | | human LOUP RT-PCR | AGACT | NO: 28 |
| junctions | hLOUP R | reverse primer for | CTGGTCAGCAGGAAA | SEQ ID |
| | | human LOUP RT-PCR | TTGGT | NO: 29 |
| | mLOUP F | forward primer for | GAAGGAACACAGGC | SEQ ID |
| | | mouse LOUP RT-PCR | CTCTCC | NO: 30 |
| | mLOUP R | reverse primer for | GAGACCATGCCAGTC | SEQ ID |
| | | mouse LOUP RT-PCR | TGGTT | NO: 31 |
| Primers to | hLOUP F | forward primer for | GGCTTCAGCCTCCCT | SEQ ID |
| clone LOUP | | human LOUP RT-PCR, | AGACT | NO: 28 |
| fragments used | | spliced LOUP | | |
| to generate | hLOUP R | reverse primer for | CTGGTCAGCAGGAAA | SEQ ID |
| RNA standard | | human LOUP RT-PCR, | TTGGT | NO: 29 |
| curve | | spliced LOUP | | |
| | hLOUP F | forward primer for | GGCTTCAGCCTCCCT | SEQ ID |
| | | human LOUP RT-PCR, | AGACT | NO: 28 |
| | | unspliced LOUP | | |
| | hLOUP R1 | reverse primer for | TCACCACAGGAAGCA | SEQ ID |
| | | human LOUP RT-PCR, | TGTGT | NO: 32 |
| | | unspliced LOUP | | |
| Strand- | hLOUP F | forward primer for | GGCTTCAGCCTCCCT | SEQ ID |
| specific | | human LOUP RT-PCR | AGACT | NO: 28 |
| RT-PCR | hLOUP R | reverse primer for | CTGGTCAGCAGGAAA | SEQ ID |
| | | human LOUP RT-PCR | TTGGT | NO: 29 |
| | Un-related F | forward primer for | GGCAGAGTTCTCCCT | SEQ ID |
| | | CEBPA-AS1 RT-PCR | GTGC | NO: 33 |
| | Un-related R | reverse primer for | GTGGAGTCGCCGATT | SEQ ID |
| | | CEBPA-AS1 RT-PCR | TTT | NO: 34 |
| qPCR | hLOUP F | forward primer for | GGCTTCAGCCTCCCT | SEQ ID |
| | | mature human LOUP | AGACT | NO: 28 |
| | hLOUP R | reverse primer for | CTGGTCAGCAGGAAA | SEQ ID |
| | | mature human LOUP | TTGGT | NO: 29 |
| | hLOUP F1 | forward primer for | GTGGGCTAGTCTGTG | SEQ ID |
| | | immature human LOUP | GAAGG | NO: 35 |
| | hLOUP R | reverse primer for | CTGGTCAGCAGGAAA | SEQ ID |
| | | immature human LOUP | TTGGT | NO: 29 |
| | mLOUP F | forward primer for | GAAGGAACACAGGC | SEQ ID |
| | | mouse mature LOUP | CTCTCC | NO: 30 |
| | mLOUP R | reverse primer for | TTTCTGGCCTTGAAC | SEQ ID |
| | | mouse mature LOUP | TGACA | NO: 31 |
| | mLOUP F1 | forward primer for | CCACGAGACACTATC | SEQ ID |
| | | mouse LOUP | CAGCA | NO: 36 |
| | mLOUP R1 | reverse primer for | GAGACCATGCCAGTC | SEQ ID |
| | | mouse LOUP | TGGTT | NO: 31 |
| | hMALAT1F | forward primer for | GGTCTTTGGTGGGTT | SEQ ID |
| | | human MALAT1 | GAACT | NO: 37 |
| | hMALAT1R | reverse primer for | TTCCCACCCAGCATT | SEQ ID |
| | | human MALAT1 | ACAGT | NO: 38 |
| | mMALAT1F | forward primer for | GGTCTTTGGTGGGTT | SEQ ID |
| | | mouse MALAT1 | GAACT | NO: 37 |
| | mMALAT1R | reverse primer for | TTCCCACCCAGCATT | SEQ ID |
| | | mouse MALAT1 | ACAGT | NO: 38 |
| | RPPH1 F | forward primer for | CTAACAGGGCTCTCC | SEQ ID |
| | | human RPPH1 | CTGAG | NO: 39 |
| | RPPH1 R | reverse primer for | CAGCCATTGAACTCA | SEQ ID |
| | | human RPPH1 | CTTCG | NO: 40 |
| | mRPS18 F | forward primer for | CGGAAAATAGCCTTC | SEQ ID |
| | | mouse RPS18 | GCCATCAC | NO: 41 |
| | mRPS18 R | reverse primer for | ATCACTCGCTCCACC | SEQ ID |
| | | mouse RPS18 | TCATCCT | NO: 42 |
| | hPU.1 F | forward primer for | TGTTACAGGCGTGCA | SEQ ID |
| | | human PU.1 | AAATGG | NO: 43 |
| | hPU.1 R | reverse primer for | TGCGTTTGGCGTTGG | SEQ ID |
| | | human PU.1 | TATAGA | NO: 44 |
| | mm00488140_ | Taqman set for mouse | www.thermofisher.com/ | |
| | m1 Spil | Pu.1 (Purchased from | taqman-gene- | |
| | | ThermoFisher) | expression/product/Mm | |
| | | | 00488140_m1?CID=&I | |
| | | | CID=&subtype= | |
| | GAPDH F | forward primer for | GTCTCCTCTGACTTC | SEQ ID |
| | | human GAPDH | AACAGCG | NO: 45 |
| | GAPDH R | reverse primer for | ACCACCCTGTTGCTG | SEQ ID |
| | | human GAPDH | TAGCCAA | NO: 46 |
| | URE F_3C | forward primer for 3C | GTGTCTGCTCCCTAG | SEQ ID |
| | | qPCR | CTCCA | NO: 47 |
| | Taqman_3C | Taqman probe for 3C | ATGGCGTGTGGTCAC | SEQ ID |
| | | qPCR | CCAGA | NO: 48 |
| | -8K R_3C | reverse primer for | GACAGTGCTACATGG | SEQ ID |
| | | measuring interaction | GTGTGA | NO: 49 |
| | | with the -8K region by | | |
| | | 3C Taqman qPCR | | |
| | -4K R_3C | reverse primer for | CTTTGGAGAGTCCCA | SEQ ID |
| | | measuring interaction | AGTGC | NO: 50 |
| | | with the -4K region by | | |
| | | 3C Taqman qPCR | | |
| | PrPr R_3C | reverse primer for | GAGCCATAGCGGTG | SEQ ID |
| | | measuring interaction | AGTACG | NO: 51 |
| | | with the PrPr region by | | |
| | | 3C Taqman qPCR | | |
| | Intergenic R_3C | reverse primer for | TTCTCCCTGGAGAGA | SEQ ID |
| | | measuring interaction | CCTCA | NO: 52 |
| | | with intergenic region | | |
| | | by 3C Taqman qPCR | | |
| | MYBPC3 R_3C | reverse primer for | GGTGTGCACCACCAT | SEQ ID |
| | | measuring interaction | ACTTG | NO: 53 |
| | | with MYBPC3 gene by | | |
| | | 3C Taqman qPCR | | |
| | URE F | forward primer for | GCCATGAAATGCTCT | SEQ ID |
| | | detecting URE by ChIRP | GCTCT | NO: 54 |
| | URE R | reverse primer for | CCTAGCCCTTGGAAG | SEQ ID |
| | | detecting URE by ChIRP | GAGAC | NO: 55 |
| | PrPr F | forward primer for | CAGCCCTTTGAGCAC | SEQ ID |
| | | detecting PrPr by ChIRP | CAC | NO: 56 |
| | PrPr R | reverse primer for | GAAGGGCCTGCCGC | SEQ ID |
| | | detecting PrPr by ChIRP | TGGGAGATAG | NO: 57 |
| | ACTBpro_F | forward primer for | AAAGGCAACTTTCGG | SEQ ID |
| | | detecting ACTB | AACGG | NO: 58 |
| | | promoter by ChIRP | | |
| | ACTBpro_R | reverse primer for | TTCCTCAATCTCGCT | SEQ ID |
| | | detecting ACTB | CTCGC | NO: 59 |
| | | promoter by ChIRP | | |
| | LOUP fRIP F1 | forward primer for LOUP | GGAGCCCCTTGAATC | SEQ ID |
| | | fRIP qPCR, amplicon #1 | TTAGG | NO: 60 |
| | LOUP fRIP R1 | reverse primer for LOUP | AAAGCAGGACAGGA | SEQ ID |
| | | fRIP qPCR, amplicon #1 | AAGCAA | NO: 61 |
| | LOUP fRIP F2 | forward primer for LOUP | CAGGTGGCACACATC | SEQ ID |
| | | fRIP qPCR, amplicon #2 | CATAG | NO: 62 |
| | LOUP fRIP R2 | reverse primer for LOUP | CATGCTTGGCCAGTT | SEQ ID |
| | | fRIP qPCR, amplicon #2 | CTTTT | NO: 63 |
| | LOUP fRIP F3 | forward primer for LOUP | TCAACAGATGGCTGT | SEQ ID |
| | | fRIP qPCR, amplicon #3 | CTTGG | NO: 64 |
| | LOUP fRIP R3 | reverse primer for LOUP | TCAGAAGCCTCATCC | SEQ ID |
| | | fRIP qPCR, amplicon #3 | CCTTA | NO: 65 |
| | URE ChIP F | forward primer for URE | CTGTGGTAATGGGCT | SEQ ID |
| | | ChIP qPCR | GTTGG | NO: 66 |
| | URE ChIP R | reverse primer for URE | CTCTGGGCAGGGTC | SEQ ID |
| | | ChIP qPCR | ACAG | NO: 67 |
| | PrPr ChIP F | forward primer for PrPr | GGCTGACTCCAGAAA | SEQ ID |
| | | ChIP qPCR | GTGGA | NO: 68 |
| | PrPr ChIP R | reverse primer for PrPr | GGGAGAACGTGTAG | SEQ ID |
| | | ChIP qPCR | CTCTGC | NO: 69 |
| | GD ChIP F | forward primer for GENE | GGCTAATCCTCTATG | SEQ ID |
| | | DESERT ChIP qPCR | GGAGTCTGTC | NO: 70 |
| | GD ChIP R | reverse primer for GENE | CCAGGTGCTCAAGGT | SEQ ID |
| | | DESERT ChIP qPCR | CAACATC | NO: 71 |
| Identification | P5_1F | P5-splinkerette adapter | AATGATACGGCGACC | SEQ ID |
| of | | | ACCGAGATCTACACT | NO: 72 |
| 5′ End of LOUP | | | CTTTCCCTACACGAC | |
| trancript | | | GCTCTTCCGATCT | |
| | P5_2F | P5 primer | AATGATACGGCGACC | SEQ ID |
| | | | ACCGAGATCT | NO: 73 |
| | hLOUP R | LOUP-specific nested | CTGGTCAGCAGGAAA | SEQ ID |
| | | primer #1 | TTGGT | NO: 29 |
| | hLOUP R1 | LOUP-specific nested | CTGGTCAGCAGGAAA | SEQ ID |
| | | primer #2 | TTGGT | NO: 29 |
| 3′ RACE | dTA_A | Oligo dT-Anchor Primer | GACCACGCGTATCGA | SEQ ID |
| Primers | | mix #1 | TGTCGACTTTTTTTTT | NO: 74 |
| | | | TTTTTTTA | |
| | dTA_C | Oligo dT-Anchor Primer | GACCACGCGTATCGA | SEQ ID |
| | | mix #2 | TGTCGACTTTTTTTTT | NO: 75 |
| | | | TTTTTTTC | |
| | dTA_G | Oligo dT-Anchor Primer | GACCACGCGTATCGA | SEQ ID |
| | | mix #3 | TGTCGACTTTTTTTTT | NO: 76 |
| | | | TTTTTTTG | |
| | hLOUP F | forward primer #1 for | GGCTTCAGCCTCCCT | SEQ ID |
| | | LOUP 3′ RACE | AGACT | NO: 28 |
| | hLOUP F_a | forward primer #2 for | CTGTCTCCTTCCAAG | SEQ ID |
| | | LOUP 3′ RACE | GGCTA | NO: 77 |
| | hLOUP F_b | forward primer #3 for | CAGGTGGCACACATC | SEQ ID |
| | | LOUP 3′ RACE | CATAG | NO: 62 |
| | Anchor R | Anchor reverse primer | GACCACGCGTATCGA | SEQ ID |
| | | | TGTCGAC | NO: 78 |
| ChIRP probes | LOUP_01 | LOUP-tiling oligo | AAGGAGACAGGAGT | SEQ ID |
| | | | CTAGGG/3BioTEG | NO: 79 |
| | LOUP_02 | LOUP-tiling oligo | TCTGGTCAGCAGGAA | SEQ ID |
| | | | ATTG/3BioTEG | NO: 80 |
| | LOUP_03 | LOUP-tiling oligo | CAGAGCAAAAGAGG | SEQ ID |
| | | | GGCAGA/3BioTEG | NO: 81 |
| | LOUP_04 | LOUP-tiling oligo | AGAGGAGGGACAAC | SEQ ID |
| | | | GAGGAG/3BioTEG | NO: 82 |
| | LOUP_05 | LOUP-tiling oligo | CAGGACAAGAGGTG | SEQ ID |
| | | | AGGAGG/3BioTEG | NO: 83 |
| | LOUP_06 | LOUP-tiling oligo | GATCTCACATCACCA | SEQ ID |
| | | | AGACA/3BioTEG | NO: 84 |
| | LOUP_07 | LOUP-tiling oligo | CGGTTTGGTAATCCA | SEQ ID |
| | | | TAACC/3BioTEG | NO: 85 |
| | LOUP_08 | LOUP-tiling oligo | AGTACATCAGAAGCC | SEQ ID |
| | | | TCATC/3BioTEG | NO: 86 |
| | LOUP_09 | LOUP-tiling oligo | AGGGTCAATAACCTC | SEQ ID |
| | | | TGGA/3BioTEG | NO: 87 |
| | LOUP_10 | LOUP-tiling oligo | GCTCCAGGAGAAGG | SEQ ID |
| | | | AAGATA/3BioTEG | NO: 88 |
| | LOUP_11 | LOUP-tiling oligo | TGCTGGTTGTAAGCA | SEQ ID |
| | | | AGGA/3BioTEG | NO: 89 |
| | LOUP_12 | LOUP-tiling oligo | GCAAAGCAGGACAG | SEQ ID |
| | | | GAAAGC/3BioTEG | NO: 90 |
| | LOUP_13 | LOUP-tiling oligo | GAAAGCATGTCTGGC | SEQ ID |
| | | | TGAG/3BioTEG | NO: 91 |
| | LOUP_14 | LOUP-tiling oligo | GGTACACTTGGTCTC | SEQ ID |
| | | | AAAG/3BioTEG | NO: 92 |
| | LacZ_01 | LacZ-tiling oligo | CCAGTGAATCCGTAA | SEQ ID |
| | | | TCATG/3BioTEG | NO: 93 |
| | LacZ_02 | LacZ-tiling oligo | GTAGCCAGCTTTCAT | SEQ ID |
| | | | CAACA/3BioTEG | NO: 94 |
| | LacZ_03 | LacZ-tiling oligo | ATCTTCCAGATAACT | SEQ ID |
| | | | GCCGT/3BioTEG | NO: 95 |
| | LacZ_04 | LacZ-tiling oligo | ATAATTTCACCGCCG | SEQ ID |
| | | | AAAGG/3BioTEG | NO: 96 |
| | LacZ_05 | LacZ-tiling oligo | TTCATCAGCAGGATA | SEQ ID |
| | | | TCCTG/3BioTEG | NO: 97 |
| | LacZ_06 | LacZ-tiling oligo | TGATCACACTCGGGT | SEQ ID |
| | | | GATTA/3BioTEG | NO: 98 |
| | LacZ_07 | LacZ-tiling oligo | AAACGGGGATACTGA | SEQ ID |
| | | | CGAAA/3BioTEG | NO: 99 |
| | LacZ_08 | LacZ-tiling oligo | GTTATCGCTATGACG | SEQ ID |
| | | | GAACA/3BioTEG | NO: 100 |
| | LacZ_09 | LacZ-tiling oligo | TGTGAAAGAAAGCCT | SEQ ID |
| | | | GACTG/3BioTEG | NO: 101 |
| | LacZ 10 | LacZ-tiling oligo | GTAATCGCCATTTGA | SEQ ID |
| | | | CCACT/3BioTEG | NO: 102 |
| DNA pull-down | URE Runx1 wt F | forward URE oligo | [Btn]AGGGTGTGGCA | SEQ ID |
| assay | | containing Runx1 | GGTGTGGACGT | NO: 103 |
| | | wildtype binding site | | |
| | URE Runx1 wt | reverse URE oligo | ACGTCCACACCTGCC | SEQ ID |
| | R | containing Runx1 | ACACCCT | NO: 104 |
| | | wildtype binding site | | |
| | URE Runx1 mt | forward URE oligo | [Btn]AGGCTCTCACAG | SEQ ID |
| | F | containing Runx1 mutant | CTCTCAACGT | NO: 105 |
| | | binding site | | |
| | URE Runx1 mt | reverse URE oligo | ACGTTGAGAGCTGTG | SEQ ID |
| | R | containing Runx1 mutant | AGAGCCT | NO: 106 |
| | | binding site | | |
| | PrPr Runx1 wt F | forward PrPr oligo | [Btn]CAGTGGTGTGG | SEQ ID |
| | | containing Runx1 | CAGAGCTAC | NO: 107 |
| | | wildtype binding site | | |
| | PrPr Runx1 wt R | reverse PrPr oligo | GTAGCTCTGCCACAC | SEQ ID |
| | | containing Runx1 | CACTG | NO: 108 |
| | | wildtype binding site | | |
| | PrPr Runx1 mt F | forward PrPr oligo | [Btn]CAGTGCTCTCAC | SEQ ID |
| | | containing Runx1 mutant | AGAGCTAC | NO: 109 |
| | | binding site | | |
| | PrPr Runx1 mt | reverse PrPr oligo | GTAGCTCTGTGAGAG | SEQ ID |
| | R | containing Runx1 mutant | CACTG | NO: 110 |
| | | binding site | | |
| Northern blot | hLOUP F | forward primer for | GGCTTCAGCCTCCCT | SEQ ID |
| probe | | northern blot probe of | AGACT | NO: 28 |
| | | LOUP | | |
| | hLOUP R | reverse primer for | CTGGTCAGCAGGAAA | SEQ ID |
| | | northern blot probe of | TTGGT | NO: 29 |
| | | LOUP | | |
| Oligos for | #D1 sgRNA fwd | forward single guide | CACCGCAGGTGGTC | SEQ ID |
| cloning sgRNA | | RNA sequence insert for | TCAGAGGTCGG | NO: 111 |
| into | | LOUP #D1 sgRNA | | |
| CRISPR/Cas9 | #D1 sgRNA rev | reverse single guide | AAACCCGACCTCTGA | SEQ ID |
| plasmids | | RNA sequence insert for | GACCACCTGC | NO: 112 |
| | | LOUP #D1 sgRNA | | |
| | #D2 sgRNA fwd | forward single guide | CACCgCACAAGATCA | SEQ ID |
| | | RNA sequence insert for | GGTAACAAGT | NO: 113 |
| | | LOUP #D2 sgRNA | | |
| | #D2 sgRNA rev | reverse single guide | AAACACTTGTTACCT | SEQ ID |
| | | RNA sequence insert for | GATCTTGTGC | NO: 114 |
| | | LOUP #D2 sgRNA | | |
| | **control | forward single guide | AAACCCCACCAATAT | SEQ ID |
| | sgRNA fwd | RNA sequence insert for | CAGTAATACC | NO: 115 |
| | | CRISPR/Cas9 non- | | |
| | | targeting control | | |
| | **control | reverse single guide | AAACCCCACCAATAT | SEQ ID |
| | sgRNA rev | RNA sequence insert for | CAGTAATACC | NO: 116 |
| | | CRISPR/Cas9 non- | | |
| | | targeting control | | |
| TA strata | #D1 LOUP TA | forward primer to amplify | GAGCTGAGAGCCCA | SEQ ID |
| cloning of | fwd | amplicon containing #D1 | GAAGAA | NO: 117 |
| LOUP | | LOUP CRISPR/Cas9 | | |
| CRISPR/Cas9 | | target site | | |
| targeted | #D1 LOUP TA | reverse primer to amplify | CTCGGCCTTCTCGCA | SEQ ID |
| alleles | rev | amplicon containing #D1 | AAGA | NO: 118 |
| | | LOUP CRISPR/Cas9 | | |
| | | target site | | |
| | #D2 LOUP TA | forward primer to amplify | GACAGTGCTACATGG | SEQ ID |
| | fwd | amplicon containing #D2 | GTGTGA | NO: 119 |
| | | LOUP CRISPR/Cas9 | | |
| | | target site | | |
| | #D2 LOUP TA | reverse primer to amplify | AGGGACAACGAGGA | SEQ ID |
| | rev | amplicon containing #D2 | GGTTTT | NO: 120 |
| | | LOUP CRISPR/Cas9 | | |
| | | target site | | |
| Oligos for | #A1 sgRNA fwd | single guide RNA | CACCGAGAACTCCTA | SEQ ID |
| cloning sgRNA | | sequence insert for | GCGGGACACT | NO: 121 |
| into | | CRISPR/dCas9-VP64 | | |
| CRISPR/dCas9- | | targeting LOUP | | |
| VP64 | | promoter region | | |
| | #A1 sgRNA rev | single guide RNA | AAACAGTGTCCCGCT | SEQ ID |
| | | sequence insert for | AGGAGTTCTC | NO: 122 |
| | | CRISPR/dCas9-VP64 | | |
| | | targeting LOUP | | |
| | | promoter region | | |
| | #A2 sgRNA fwd | single guide RNA | CACCGATGGCTGAG | SEQ ID |
| | | sequence insert for | GTTGATGGTTG | NO: 123 |
| | | CRISPR/dCas9-VP64 | | |
| | | targeting LOUP | | |
| | | promoter region | | |
| | #A2 sgRNA rev | single guide RNA | AAACCAACCATCAAC | SEQ ID |
| | | sequence insert for | CTCAGCCATC | NO: 124 |
| | | CRISPR/dCas9-VP64 | | |
| | | targeting LOUP | | |
| | | promoter region | | |
| Oligos for | Sp6R1 fwd | forward primer to amplify | AATTTAGGTGACACT | SEQ ID |
| cloning LOUP | | LOUP R1-S | ATAGAACTACAGGTG | NO: 125 |
| fragments into | | | GCACACATCCA | |
| pSCAmpKan to | R1 rv | reverse primer to amplify | GCTGGAGTGCAATG | SEQ ID |
| use in RNAP | | LOUP R1-S | GCGTGATC | NO: 126 |
| assays | Sp6R1-AS fwd | forward primer to amplify | AATTTAGGTGACACT | SEQ ID |
| | | LOUP R1-AS | ATAGATCTTGGCCCA | NO: 127 |
| | | | CTGTAGCCT | |
| | R1-AS rev | reverse primer to amplify | ACTACAGGTGGCACA | SEQ ID |
| | | LOUP R1-AS | CATCCAT | NO: 128 |
| | Sp6R2 fwd | forward primer to amplify | AATTTAGGTGACACT | SEQ ID |
| | | LOUP R2-S | ATAGAATACAATAAT | NO: 129 |
| | | | TAGCTGGGCGTG | |
| | R2 rv | reverse primer to amplify | GTTTCGCTCTTGTTG | SEQ ID |
| | | LOUP R2-S | CCCAGGCTGG | NO: 130 |
| | RR fwd | forward primer to amplify | AATTTAGGTGACACT | SEQ ID |
| | | LOUP RR | ATAGAACAACCTCTA | NO: 131 |
| | | | CGGAAAAGAGTATG | |
| | RR rev | reverse primer to amplify | CCTTTCTTCTTTTCTC | SEQ ID |
| | | LOUP RR | TCTTTTTCTTTTTC | NO: 132 |

Italic amino acid residues are 5′ overhangs for cloning into CRISPR/Cas9 plasmids (pLVx U6se EF1a sfPac); **Addgene control oligo sequence www.addgene.org/80248/; Underlined amino acid residues are 5′ overhangs for cloning into CRISR/dCas9 plasmids (pXPR_502); Bold amino acid residues are 5′ overhangs containing sp6 promoter for in vitro transcription

Generation of CRISPR Activation Cells (CRISPRa)

sgRNAs targeting the 500 bp upstream region of LOUP's transcriptional start site were designed using Cas-Designer (Park et al., 2015, supra). The sgRNAs were then cloned into the pXR502 plasmid as previously described (Ran et al., Nat. Protoc. 8: 2281-2308, 2013). K562 cells stably expressing dCas9-VP64 were generated via lentiviral delivery of dCas9-VP64-Blast (Konermann et al., Nature 517: 583-588, 2015) and Blasticidin selection. dCas9-VP64 stable cells were transduced with lentiviruses that package the sgRNA-cloned pXR502 plasmids as previously described (Ran et al., 2013, supra). After one-day post-transduction, cells were selected with puromycin for 2-3 days before collection for analysis.

### Method Details

Plasmid Transfections

K562 cells, in exponential growth, were electroporated with expression plasmids using program T16, kit V (Lonza). Electroporated cells were incubated at 37° C. overnight in a 5% CO2 incubator. The next day, cells were changed to fresh medium. Cells were harvested at 48 h after electroporation.

Cellular Fractionation, RNA Extraction, RT-PCR and qPCR Analysis

Cultured cells were washed with Phosphate-buffered saline (PBS). Total RNA was extracted with Trizol reagent (Invitrogen) or PURELINK™ RNA Mini Kit (Ambion) and treated with RNase-free DNase I (Roche) to remove contaminated genomic DNA. polyA− and polyA+ RNAs were isolated from total RNA using Poly(A)PURIST™ MAG Kit (Ambion) following manufactural procedure. Isolation of RNA from subcellular fractions was performed as previously described (Lee et al., Cell 164: 69-80, 2016) with modifications. Briefly, cells were lysed in cytosolic lysis solution (10 mM HEPES pH 7.9, 1.5 mM MgCl2, 10 mM KCl, 0.5% NP40, 1 mM DTT plus protease and RNase inhibitors) for 10 min on ice. After centrifugation, the supernatant was collected as the cytoplasmic fraction for cytosol RNA isolation. After washing in cytosolic lysis solution, nuclear pellet was used for nuclear RNA isolation. To collect nucleoplasm and chromatin fractions, nuclear pellet was further lysed with nuclear lysis solution (20 mM HEPES pH 7.9, 1.5 mM MgCl2, 450 nM NaCl, 0.2 mM EDTA, 25% glycerol, 1 mM DTT, plus protease and RNase inhibitors). After centrifugation, nuclear-soluble fraction (nucleoplasm) was collected as supernatant and chromatin-associated fraction was collected as pellet. RNAs from collected fractions were extracted with Trizol reagent and treated with RNase-free DNase I (Roche).

For RT-PCR, RNA was reverse-transcribed by using SuperScript® III Reverse Transcriptase (Invitrogen). Red Taq Pro Complete (Denville Scientific) was used to amplify designated amplicons. For qPCR assays, cDNA was generated by QuantiTect Rev. Transcription Kit (Qiagen) which also includes additional DNA contamination removal. iQ SYBR Green Supermix (Biorad) was used for PCR quantitation in a RotorGene cycler (Corbett). Relative quantification was performed using the ddCt method. To calculate LOUP transcript numbers per cell, LOUP DNA fragments amplified by RT-PCR from HL-60 cDNA were cloned into pSCAmpKan plasmid (Agilent). LOUP RNA fragments were in vitro-transcribed by using MAXIscript™ Transcription Kit (Ambion). The RNA fragments were used to generate a standard curve for absolute quantification in qRT-PCR assays.

Fluorescence-Activated Cell Sorting and Analysis

Cell populations were isolated for RNA extraction as previously described (Zhang et al., Cancer Cell 24: 575-588, 2013). Briefly, mononuclear cells were isolated bone marrow, spleen and peripheral blood after lysing red blood cell with ACK lysis buffer (Zhang et al., Immunity 21: 853-863, 2004). Single cell suspension was stained with fluorochrome-conjugated antibodies (Biolegend and eBioscience) and FACS-sorted based on the following markers. LT-HSC: Lin-c-Kit+Sca-1+CD150+CD48−; ST-HSC: Lin-c-Kit+Sca-1+CD150−CD48+; LMPP: Lin-c-Kit+Sca-1+CD34+Flt3+; MEP: Lin-c-Kit+Sca-1-CD34−CD16/32−; CMP: Lin-c-Kit+Sca-1-CD34+CD16/32−; GMP: Lin-c-Kit+Sca-1-CD34+CD16/32+; Mac/Gr1:Mac1+Gr1+.

Myeloid surface marker staining and FACS analysis were performed following previously described procedure (Mueller et al., Blood 107: 3330-3338, 2006). Cells were stained with PACBLUE-CD11b (BioLegend). Stained cells were analyzed using LSRII flow cytometer (BD Biosciences) and FlowJo software (Tree Star).

Transcript Mapping by P5-Linker Ligation and 3′ RACE

The 5′ end of LOUP transcript was identified using P5-linker ligation method as described previously (Melo et al., Mol. Cell 49: 524-535, 2013). Briefly, single-stranded cDNAs were generated from HL-60 polyA+ RNA by using SuperScript III reverse transcriptase (Life Technologies) with LOUP-specific nested primer #1. Double-strand cDNAs were then synthesized from single-stranded cDNA using SUPERSCRIPT™ Double-Stranded cDNA Synthesis Kit (Life Technologies) and blunt-ended by NEBNext End Repair Enzym Module (New England Biolabs). After purification, these cDNAs were ligated with P5-splinkerette adapter and purified. All purification steps were done by using QiAquick PCR Purification Kit (QIAGEN). Ligated products were then purified and used as templates for PCR with P5 primer and LOUP-specific nested primers #1 and #2 with Phusion Hot Start DNA polymerase (Finnzymes). P5-linker ligation products were gel purified using QIAgen Gel Extraction Kit (QIAGEN) and sub-cloned into pSCAmpKan vector and transformed into competent bacteria using StrataClone Blunt PCR Cloning Kit (Agilent). 3′RACE assay was performed using 2nd Generation 5/3′ RACE Kit (Roche) according to manufacturer's instruction. Briefly, cDNA was generated from HL-60 polyA₊ RNA using oligo dT-anchor primer mix. Overlapping RACE products were then amplified from cDNA using anchor primer and LOUP-specific primers. RACE products were sub-cloned into pSCAmpKan vector and transformed into competent bacteria using StrataClone Cloning Kit (Agilent). Plasmids containing p5-linker and RACE products were purified from bacteria, sequenced, and assembled.

Northern Blotting

10 ug polyA− and polyA+ RNAs were dissolved and heat denatured in sample buffer containing formamide, MOPS and formaldehyde. Denatured RNAs were separated on a 1% denaturing agarose gel containing formaldehyde, MOPS and EtBr and transferred to Brightstar-plus positively charged nylon membrane (Life Technologies). LOUP probe was PCR amplified with primers described in Table 3 (Northern blot probe). PCR product was sub-cloned into cloned into pSCAmpKan vector using StrataClone PCR Cloning Kit (Agilent). Probe sequence was verified by Sanger sequencing. Probe was released from the vector by restriction enzyme digestion and gene purification. Probe was radiolabeled using the Random Primed DNA Labeling Kit (Roche). Northern blot was performed with EXPRESSHYB™ Hybridization Solution (Clontech) following manufacture protocol

Quantitative Chromosome Conformation Capture (3C-qPCR)

3C-qPCR experiments were performed by adapting described methods (Deng and Blobel, Methods Mol. Biol. 1468: 51-62, 2017; Hagege et al., Nat. Protoc. 2: 1722-1733, 2007; Staber et al., 2013, supra). Briefly, 1×10⁶ cells were crosslinked using 1% formaldehyde in PBS at room temperature for 10 min. Crosslinking reaction was stopped by adding 0.125 M Glycine and incubated for 5 min at room temperature followed by 15 min on ice. Crosslinked cells were then washed with ice-cold PBS and lysed in 3C lysis buffer (10 mM Tris-HCl, pH 8.0; 10 mM NaCl; Igepal CA-630 0.2% (vol/vol); 1× protease inhibitor cocktail (Sigma)) with 15 Dounce homogenizer strokes. After centrifugation, nuclear pellets were washed in 1× restriction enzyme buffer before being lysed with 0.1% SDS in 1× restriction enzyme buffer at 65° C. for 10 min. After incubation, chromatin solution was supplemented with 1% Triton X-100 and digested by ApoI restriction enzyme (New England Biolabs) at 37° C. overnight with rotation. The following day, 1.5% SDS was added to the reaction and enzyme activity was inhibited by incubating at 65° C. for 30 min. Nearby DNA ends of digested chromatin were joined by T4-ligase (New England Biolabs) at 16° C. for 2 h. Bound proteins including histones were removed by proteinase K at 65° C. overnight. DNA library were extracted by phenol/chloroform using phase-lock gel tubes (SPRIME) and ethanol precipitation. RNA was removed by incubating 3C libraries with RNase A (Lucigen) at 37° C. for 15 min. TaqMan real-time PCR quantifications of ligation products were performed, using primers and probes as documented in Table 3.

Chromatin Isolation by RNA Purification (ChIRP)

ChIRP assays were performed as described (Chu et al., J. Vis. Exp. 25(61): pii: 3912, Trimarchi et al., Cell 158: 893-606, 2014) with additional modifications. Briefly, to preserve RNA-Chromatin interactions, cells were first crosslinked with 2 mM EGS at room temperature for 45 washing cells with ice-cold PBS, cells were further crosslinked with 3% paraformaldehyde for 15 min at room temperature after ice-cold PBS washing. The crosslinking reaction was quenched with 0.125 M glycine for 5 min at room temperature. Crosslinked cells were washed in ice-cold PBS and lysed in sonication buffer (20 mM Tris pH 8, 150 mM NaCl, 0.1% SDS, 1% Triton-X, 2 mM EDTA, 1 mM PMSF) supplemented with COMPLETE™, Mini Protease Inhibitor Cocktail (Sigma-Aldrich) and SUPERase In RNase Inhibitor (Invitrogen). After sonication and centrifugation, supernatant containing sheared chromatin was collected and incubated with biotinylated anti-sense DNA tiling probes in hybridization buffer (750 mM NaCl, 1% Triton, 0.1% SDS, 50 mM Tris-CI pH 7.0, 1 mM EDTA, 15% formamide, 1 mM PMSF) supplemented with COMPLETE™, Mini Protease Inhibitor Cocktail and SUPERase In RNase Inhibitor. Hybridized chromatin fragments were captured using DYNABEADS™ MYONE™ Streptavidin C1 (Invitrogen). From the isolated chromatin pellet, chromatin-bound RNA was extracted by Trizol reagent to quantitate chromatin-bound LOUP by RT-qPCR, and DNA was isolated to quantitate enrichment of the URE and the PrPr by qPCR. Probes used in the ChIRP assay were designed by using the online probe designer at singlemoleculefish.com and are listed in Table 3 (ChIRP probes).

DNA Pull-Down Assay (DNAP)

DNAP was performed as described previously with minor modifications (Trinh et al., Oncogene 30: 2718-2729, 2011). Briefly, nuclear extract was pre-cleared with DYNABEADS™ MYONE™ Streptavidin C1 for 30 min at 4° C. then incubated overnight with biotinylated oligonucleotide in binding buffer (10 mM HEPES pH 7.9; 100 mM KCl, 5 mM MgCl2, 1 mM EDTA, 10% glycerol, 1 mM DTT, 0.5% NP-40, 1 mM DTT) supplemented with 1× protease inhibitor cocktail (Sigma-Aldrich). Beads were washed with binding buffer then added to the binding reaction. After 1 h incubation, beads were washed five times with binding buffer. DNA-bound proteins were eluted from beads and subjected to SDS-PAGE and immunoblotting.

RNA Pull-Down Assay (RNAP) and RNA-Protein Interaction Prediction

RNAP were performed essentially as described previously (Tsai et al., Science 329: 689-693, 2010) with few modifications. Briefly, biotinylated RNA was in vitro-transcribed using the MAXISCRIPT™ Transcription Kit (Ambion). DNA template was removed by DNAsel treatment and transcribed RNA was purified using RNeasy Mini Kit (QIAGEN). Purified RNA was denatured by heating to 90° C. for 2 min following incubation on ice for 2 min in RNA structure buffer (10 mM Tris pH 7, 0.1 M KCl, 10 mM MgCl2). Denatured RNA was then shifted to room temperature for 20 min to form proper secondary structure. Nuclear extract was treated with RNase-free DNase I (Roche) to remove genomic DNA and pre-cleared with DYNABEADS™ MYONE™ Streptavidin C1 or Streptavidin agarose beads (Invitrogen) in binding buffer I (150 mM KCl, 25 mM Tris pH 7.4, 0.5 mM DTT, 0.5% NP40, 1 mM PMSF) supplemented with COMPLETE™, Mini Protease Inhibitor Cocktail and SUPERase In RNase Inhibitor. Pre-cleared extracts were then incubated with biotinylated RNAs in binding buffer I for 1 h. Beads were washed with binding buffer I then added to the binding reaction. After 1 h incubation, beads were washed five times with binding buffer I. RNA-bound proteins were eluted from beads and subjected to SDS-PAGE and immunoblotting. For recombinant proteins, binding buffer II (50 mM Tris-CI 7.9, 10% Glycerol, 100 mM KCl, 5 mM MgCl2, 10 mM β-ME 0.1% NP-40) was used.

In silico prediction of RNA-Protein interaction was performed using catRAPID Fragments algorithm where protein-RNA interaction propensities were predicted based on calculation of secondary structure, hydrogen bonding and van der Waals contributions (Bellucci et al., Nat. Methods 8: 444-445, 2011).

Formaldehyde RNA Immunoprecipitation Sequencing and qPCR (fRIP-Seq and fRIP-qPCR)

fRIP was performed following a protocol reported by Hendrickson et al. (Genome Biol. 17: 28, 2016) with modifications. Briefly, cells were crosslinked in 0.1% formaldehyde at room temperature for 10 minutes. The crosslinking reaction was quenched for 5 min at room temperature with 0.125 M glycine. Crosslinked cells were washed with ice-cold PBS. Cell pellet was lysed in RIPA lysis buffer (50 mM Tris (pH 8), 150 mM KCl, 0.1% SDS, 1% Triton-X, 5 mM EDTA, 0.5% sodium deoxycholate, 0.5 mM DTT) supplemented with protease inhibitor cocktail (Thermo Scientific) and 100 U/ml RNASEOUT™ (Invitrogen). After sonication, cell lysate was pre-cleared by incubating with DYNABEADS® Protein G (Invitrogen). Beads were then captured and removed using a magnet. Pre-cleared lysate was incubated with anti-RUNX1 antibody or IgG (Abcam) at 4° C. for 2 h before adding 50 μl of DYNABEADS® Protein G to capture antibodies. After washing, beads were kept at −20° C. or preceded to incubation with reverse-crosslinking buffer (3×PBS (without Mg or Ca), 6% N-lauroyl sarcosine, 30 mM EDTA, 15 mM DTT) supplemented with Proteinase K (Ambion) and RNASEOUT™ together with input sample. Captured RNAs were extracted by Trizol reagent. Extracted RNA was treated with DNAse from RNase-Free DNase Set (QIAGEN) then ribosomal RNA was removed using the RIBO-ZERO™ Magnetic Gold Kit (Epicentre). Treated RNA was purified using RNeasy MinElute Cleanup Kit (QIAGEN). RNA quality was determined using the RNA 6000 Pico Kit on a Bioanalyzer (Agilent). Purified RNA was used for qRT-PCR as described elsewhere and cDNA library construction with the Truseq stranded total RNA library prep kit (Illumina) according to manufacturer's protocol. The libraries were pooled together and subjected to pair-end sequencing on a Nextseq500 (Illumina) to achieve 2×40 bp reads.

Chromatin Immunoprecipitation and qPCR (ChIP-qPCR)

ChIP was performed as previously described (Mikkelsen et al., Nature 10: 553-560, 2007). Briefly, 2×10⁶ U937 cells were crosslinked with 1% formaldehyde (formaldehyde solution, freshly made: 50 mM HEPES-KOH; 100 mM NaCl; 1 mM EDTA; 0.5 mM EGTA; 11% formaldehyde) for 10 min at room temperature. The crosslinking reaction was stopped by incubating with 0.125 M glycine for 5 min at room temperature. Crosslinked cells were washed twice with ice-cold PBS (freshly supplemented with 1 mM PMSF). Cell pellet was lysed for 10 min on ice and chromatin was fragmented by sonication (25 cycles, 30-s on, 60-s off, high power, Bioruptor). Chromatin solution was incubated with 10 μg antibody overnight at 4° C. Protein A magnetic beads (New England Biolabs) was used to capture antibody-bound chromatin. After washing, chromatin was reverse-crosslinked and treated with proteinase K 65° C. Beads were then removed using a magnet and chromatin solution was treated with treatment (Epicentre) for 30 min at 37° C. ChIP DNA was extracted with Phenol:chloroform:isoamyl Alcohol 25:24:1, pH:8 (Sigma-Aldrich) and then precipitated with equal volume of isopropanol in presence of glycogen. DNA pellet was dissolved in 30 μl of TE buffer for qPCR analyses. Fold enrichment was calculated using the formula 2^{(-ΔΔCt(ChIP/IgG))}. Primer sets used for ChIP-qPCR are listed in Table 3 (qPCR).

fRIP-Seq and ChIP-Seq Data Analyses

fRIP-seq samples were de-multiplexed. Reads were deduplicated by Clumpify from the BBtools suite, (sourceforge.net/projects/bbmap/) with the parameters “dedupe spany addcount”. Adaptor quality trimming and filtering was performed by BBDuck from the BBtools suite with the parameters “ktrim=l hdist=2”. Low quality reads/bases were removed by Trimmomatic (Bolger et al., Bioinformatics 30: 2114-2120, 2014) with the parameters “LEADING:28 SLIDINGWINDOW:4:26 TRAILING:28 MINLEN:20”. The processed reads were then aligned to Human genome build 38 (hg38) by STAR aligner (Dobin et al., 2013) with the parameters “--outFilterScoreMinOverLread 0.05--outFilterMatchNminOverLread 0.05--outFilterMultimapNmax 30--outSAMprimaryFlag AllBestScore”. Coverage maps were generated using bamCoverage (part of the deepTools suite (Ramirez et al., Nucleic Acids Res. 44: W160-W165, 2016) with default parameters. Peak calling was performed using HOMER (v4.10) (Heinz et al., 2010). RUNX1 peaks with at least ten-fold over local region were selected for annotation using HOMER. Peaks were assigned to a gene locus by satisfying at least one of the following location criteria: a nearest transcription start site, on promoter, and on a transcript body. The latest version of ensemble 97 human gene CRCh38.p12 was used to retrieved gene annotation information through Biomart in Ensembl (Hunt et al., Ensembl variation resources Database (Oxford), 2018). For RUNX1 ChIP-seq data, raw reads in THP-1 cells (RUNX1: GSM2108052) were downloaded from GEO (GSE79899). Read quality were evaluated by FastQC (Andrews, Babraham Bioinformatics version 0115, 2016) before using for alignment and annotation as done for fRIP-seq data.

The following gene tracks are from published data that were deposited in GEO and processed via the Cistrome pipeline (Zheng et al., Nat. Commun. 8: 14049, 2019). H3K27Ac overlay track includes monocyte (GSM2679933), THP-1 (GSM2544236) and HL-60 (GSM2836486). H3K4Me1 overlay track includes monocyte (GSM1435532), HL-60 (GSM2836484) and THP-1 (GSM3514951). H3K4Me3 overlay track includes monocyte (GSM1435535), HL-60 (GSM945222) and THP-1 (GSM2108047). DNAse-seq overlay track includes monocyte (GSM701541) and HL-60 (GSM736595). RUNX1 ChIP-seq tracks includes CD34⁺ cells from healthy donors (GSM1097884), AML patient with FLT3-ITD and no other defined mutations (GSM1581788), AML patient with non-t(8;21) (GSM722708). The CAGE track (reverse strand and max counts) was imported from the FANTOM5 project (de Rie et al., Nat. Biotechnol. 35: 872-878, 2017).

RNA Sequencing Data Analysis (RNA-Seq)

Raw sequencing reads (FASTQ files) of the Human Body Map data set were downloaded from AEArrayExpress (E-MTAB-513). Read quality were assessed by FastQC (Andrews, 2016, supra). Reads with low-quality were trimmed by trim_galore (Krueger, Babraham Bioinformatics 045, 2017). LOUP transcript was integrated into the Ensembl human cDNA catalog GRCh38 and transcript levels were quantified against this catalog using Salmon (Patro et al., Nat. Methods 14: 417-419, 2017). For RNA-seq track visualization, the following RNA-seq raw data were downloaded from GEO: THP-1 (GSM1843218), HL-60 (GSM1843216), CD34₊ HSPC (GSM1843222), Monocyte (GSM1843224) and Jurkat (GSM2260195). Read quality was assessed by FastQC (Andrews, 2016, supra). Where necessary, reads with low-quality were trimmed by trim_galore. Coverage maps were generated using bamCoverage (part of the deepTools suite (Ramirez et al., 2016, supra) with default parameters). BigWig files were uploaded and viewed via the UCSC genome browser.

Single-Cell RNA-Seq (scRNA-Seq) Data Analyses

Raw fastq files data of mononuclear cells isolated from peripheral blood and bone marrow were obtained from the 10× Genomics public datasets repository (www.10xqenomics.com/resources/datasets/) and pooled together. Transcripts were mapped to the human transcriptome using Cell Ranger (10× Genomics) with a custom hg38 gtf containing the LOUP transcript details. Subsequent analyses were performed in R (v3.6.2) using previously published Bioconductor workflow with minor modifications (Lun et al., F1000Res 3: 2122, 2016). Filtering criteria are as bellow. First, cells with library sizes more than three median absolute deviations (MADs) below the median library or four MAD's above the median library size were filtered out. Second, cells with a total number of expressed genes (>=1 read) more than three MADs below the median total number of expressed genes or four MAD's above the median total number of expressed genes were filtered out. Third, cells with a total percent of expressed genes originating from mitochondrial DNA more than eight MADs above the median were filtered out. A doublet score was then computed to estimate the percentage of barcodes for two or more cells as previously described (Wolock et al., Cell Syst. 8, 281-291 e289, 2019). Cells with a doublet score of 0.99 were excluded. Expression of each cell was normalized by a size factor approach as previously described (Lun et al., Genome Biol. 17: 75, 2016) resulting in log₂(normalize_expression) values. Principle component and t-Distributed Stochastic Neighbor Embedding (tSNE) analyses revealed no significant batch effects to be regressed out for the samples. To account for dropouts which are being more frequent for genes with lower expression magnitude in scRNA-seq (Kharchenko et al., Nat. Methods 11: 740-742, 2014), cells with undetectable LOUP and PU.1 transcripts were referred as LOUP^{low}/PU.1^{low} and cells with detectable LOUP and PU.1 transcripts were referred as LOUP^{high}/pU.1^{high} Expression data visualization was performed using SPRING software (Weinreb et al., 2018). Briefly, a graph of cells connected to their nearest neighbors in gene expression space was determined. The data were then projected into two dimensions using a force-directed graph layout. Identity of each cell was inferred using Blueprint-Encode annotation which includes normalized expression values of 259 bulk RNA-seq samples generated from pure and defined cell populations (Consortium, Nature 489: 57-74, 2012; Martens and Stunnenberg, Haematologica 98: 1487-1489, 2013). This annotation was integrated in SingleR R package (Aran et al., Nat. Immunol. 20: 163-172, 2019). Annotated cells were grouped into major definitive cell lineages as described in the text. Gene Ontology (GO) analysis was performed using the Database for Annotation, Visualization and Integrated Discovery functional annotation tool (david.abcc.ncifcrf.gov). Significance of over-represented Gene Ontology biological processes was examined based on −log₁₀ of corrected p-values from Bonferroni-corrected modified Fisher's exact test (Dennis et al., Genome Biol. 4: P3, 2003). A list of enriched genes in LOUP^{high}/pU.1^{high} group vs. LOUP^{low}/PU.1^{low} group was generated using SPRING software (Weinreb et al., Bioinformatics 34: 1246-1248, 2018). Upregulated genes (Z-score >1) was used for GO analysis.

Prediction of Coding Potential with PhyloCSF

The cross-species multiple sequence comparisons result of 46 species (i.e., multiz100way) was downloaded from the UCSC genome browser (genome.ucsc.edu). Guided by the GENCODE gene annotation (ver. 28), the alignment of the longest isoform of each gene was extracted from alignments of cross-species multiple sequence comparisons. The alignment was analyzed by PhyloCSF (Lin et al., 2011, supra) with 58mammals mode. All possible coding reading frames on the same strand were scanned. The maximal score was used.

Quantitation and Statistical Analysis

In general, quantitation and statistical tests were performed using GraphPad Prism 8.0 software (otherwise specified in respective figure legends). Data are shown as mean±SD, n>=3. Unpaired Two-tailed Student's t-test was used to calculate statistical significance of differences between two experimental groups. p≤0.05 was considered statistically significant.

Data and Software Availability

Data are available on the Gene Expression Omnibus database under GEO Series accession number GEO: GSE140459.

### Example 2. Identification of RUNX1-Interacting RNAs at Myeloid Gene Loci

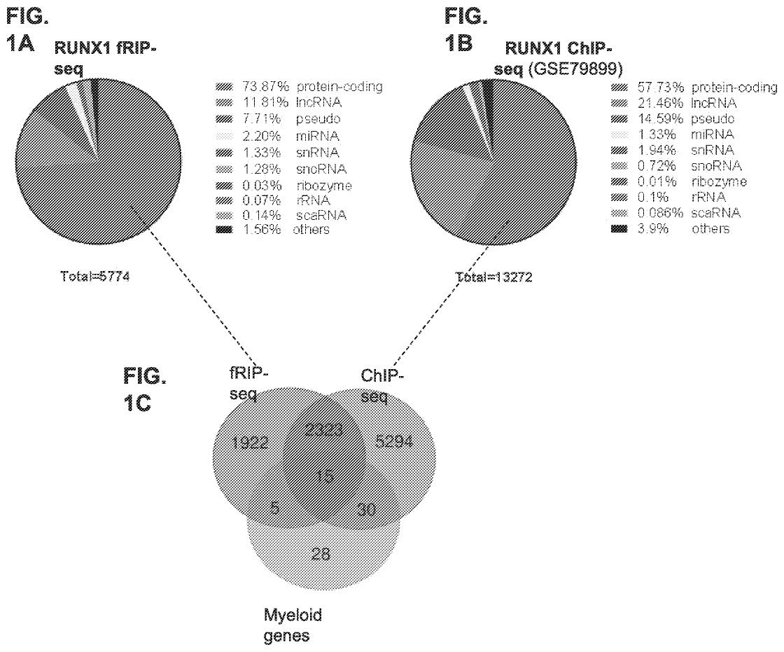

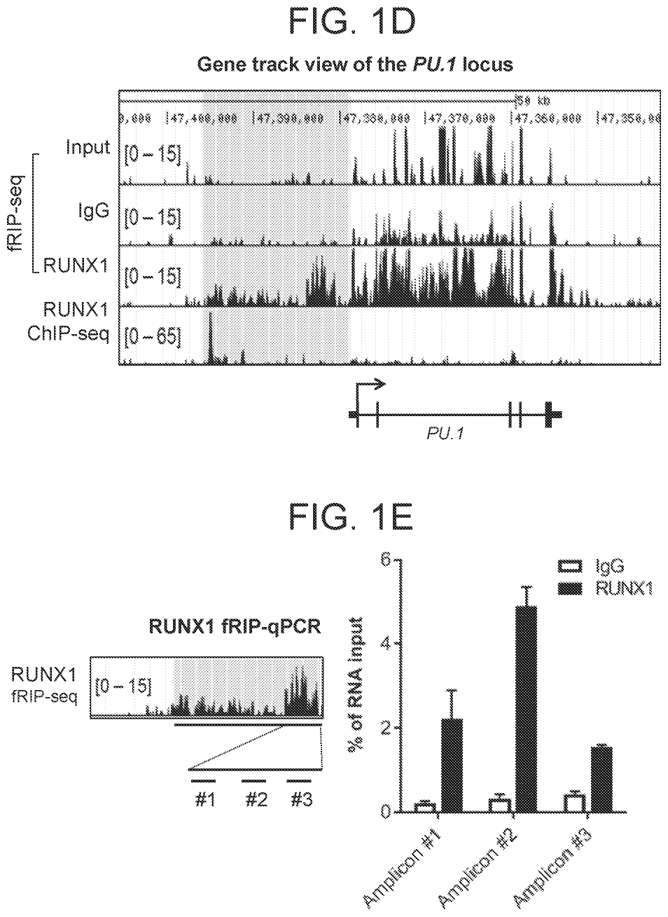

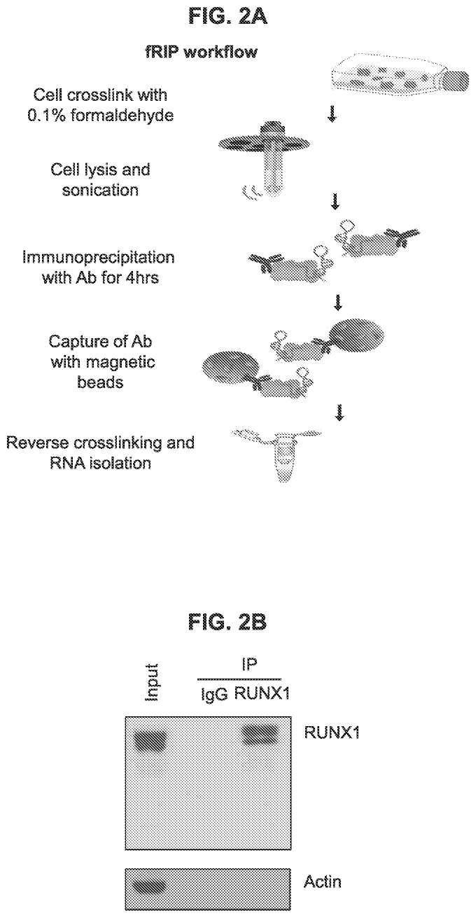

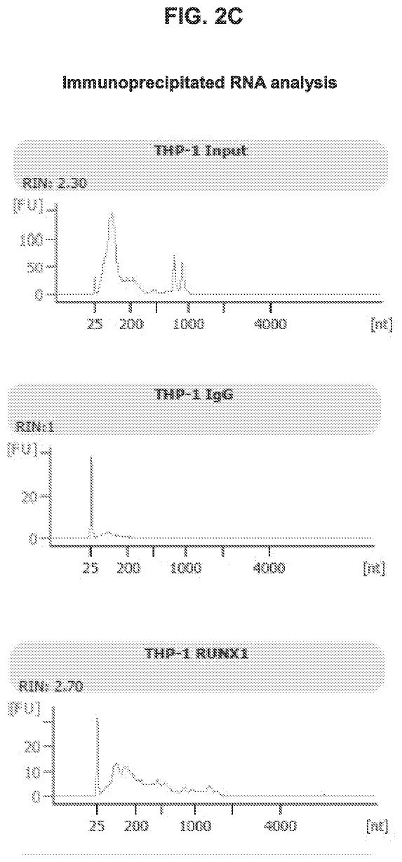

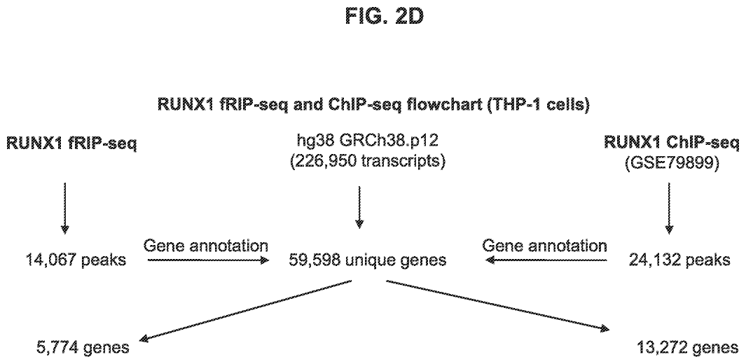

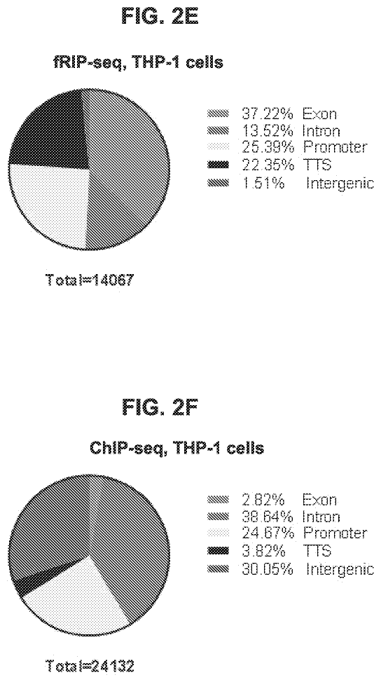

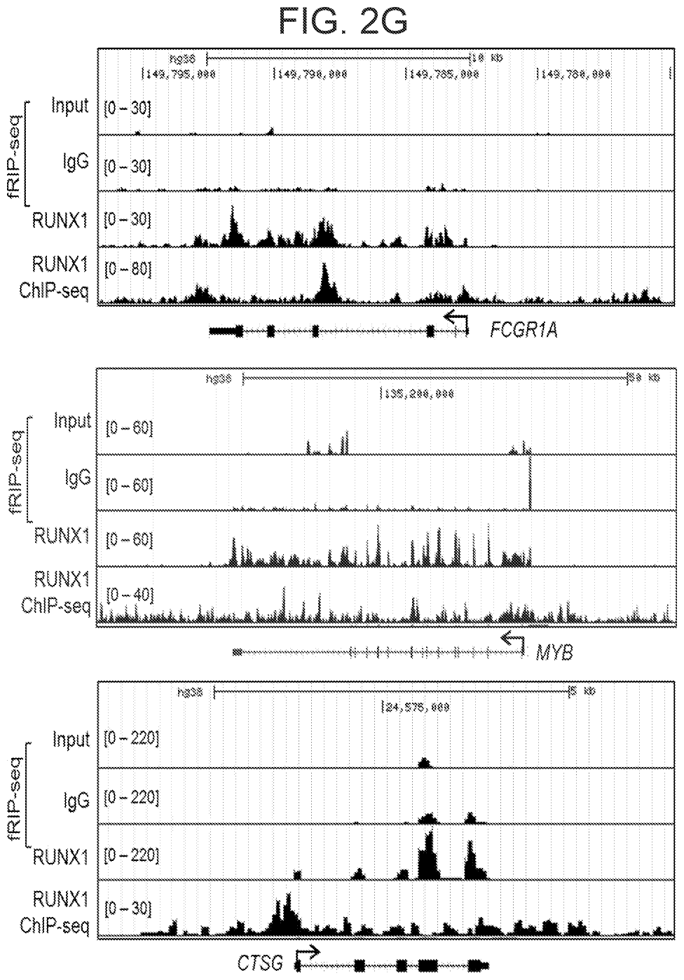

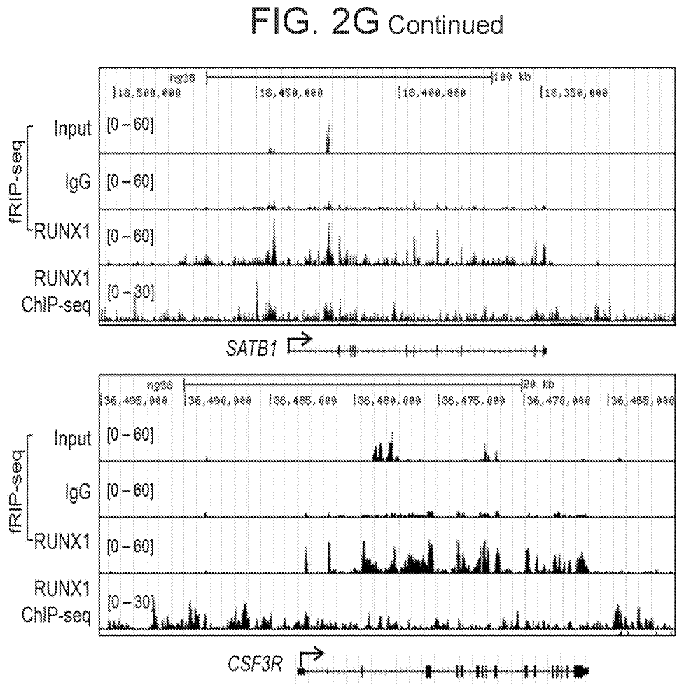

A transcriptome-wide survey for RUNX1-interacting RNAs in the monocytic cell line THP-1 was performed using formaldehyde RNA immunoprecipitation sequencing (fRIP-seq) (Hendrickson et al. Genome Biol 17: 28, 2016; Zhao et al., Mol Cell 40: 939-953, 2010). RUNX1 transcriptome was captured by anti-RUNX1 antibody (FIGS. 2A-2C) and sequenced by paired-end massively parallel sequencing. By annotating 14,067 high-confident RUNX1-fRIP peaks to the latest catalog GRCh38.p12 of Ensembl (Hunt et al., supra, 2018), which includes 59,598 genes, we identified 5,774 gene loci carrying at least one of these peaks (FIG. 2D, left). Most of the peaks located within transcript bodies and promoters (FIG. 2E). To identify genes exhibiting concurrent RUNX1-RNA and RUNX1-DNA interactions, we annotated 24,132 high-confident RUNX1-ChIP peaks to the same Ensembl catalog and identified 13,272 corresponded gene loci (FIG. 2D, right). The majority of peaks were found at intronic, promoter and intergenic regions (FIG. 2F). Because most of RUNX1-fRIP and -ChIP peaks distributed at coding gene loci (FIGS. 1A-1B), we focused our analyses on this gene group. By intersecting these genes with a list of 78 myeloid genes defined by their known roles in myeloid development or myeloid molecular markers (Table 4), we obtained 15 myeloid gene loci displaying both RUNX1-fRIP and -ChIP peaks (FIG. 1C). PU.1, a master regulator of myeloid development and a well-known transcriptional target of RUNX1 (Huang et al., 2008), was among these genes. Intriguingly, we observed RNA peaks at the upstream region of PU.1 (FIG. 1D). We further validated this observation by RUNX1 fRIP-PCR (FIG. 1E). Additional myeloid genes showing RUNX1-fRIP peaks and RUNX1-ChIP peaks were presented in FIG. 2G. The presence of previously uncharacterized RNAs, arising from the upstream region of the PU.1 locus and able to interact with RUNX1, suggests their potential role in controlling PU.1 expression through RUNX1-mediated transcriptional regulation.

TABLE 4
List of myeloid genes

| Gene | | Gene description (HUGO Gene |
|---|---|---|
| symbol | Common protein name(s) | Nomenclature) |
| ABCC8 | Sulfonylurea Receptor | ATP binding cassette subfamily C |
| | | member 8 |
| ACP5 | human purple acid phosphatase | acid phosphatase 5, tartrate resistant |
| ADGRE1 | egf-like module containing, mucin-like, | adhesion G protein-coupled receptor E1 |
| | hormone receptor-like 1 | |
| ALOX5 | Leukotriene A4 Synthase | arachidonate 5-lipoxygenase |
| ALOX5AP | MK-886-binding protein | arachidonate 5-lipoxygenase activating |
| | | protein |
| ANPEP | Myeloid Plasma Membrane Glycoprotein | alanyl aminopeptidase, membrane |
| | CD13 | |
| AZU1 | Neutrophil Azurocidin | azurocidin 1 |
| BTK | Bruton tyrosine kinase | Bruton tyrosine kinase |
| CCL2 | Monocyte Chemotactic and Activating Factor | C-C motif chemokine ligand 2 |
| CCL3 | Macrophage Inflammatory Protein 1-Alpha | C-C motif chemokine ligand 3 |
| CD14 | CD14 antigen, Myeloid Cell-Specific Leucine- | CD14 molecule |
| | Rich Glycoprotein | |
| CD36 | Glycoprotein IIIb, Leukocyte Differentiation | CD36 molecule |
| | Antigen CD36) | |
| CD68 | Macrophage Antigen CD68 | CD68 molecule |
| CEACAM8 | CD66b | CEA Cell Adhesion Molecule 8 |
| CEBPA | C/EBP-alpha | CCAAT enhancer binding protein alpha |
| CEBPB | C/EBP-beta | CCAAT enhancer binding protein beta |
| CEBPE | C/EBP-epsilon | CCAAT enhancer binding protein epsilon |
| CES1 | human monocyte/macrophage serine | carboxylesterase 1 |
| | esterase 1 | |
| CSF1R | CD115, macrophage colony-stimulating factor | colony stimulating factor 1 receptor |
| | receptor | |
| CSF2 | granulocyte-macrophage colony stimulating | colony stimulating factor 2 |
| | factor (GM-CSF) | |
| CSF2RA | CD116, alpha-GM-CSF receptor | colony stimulating factor 2 receptor alpha |
| | | subunit |
| CSF3 | Granulocyte-colony stimulating | colony stimulating factor 3 |
| | factor (G-CSF) | |
| CSF3R | CD114, granulocyte colony-stimulating factor | colony stimulating factor 3 receptor |
| | receptor (G-CSF-R) | |
| CTSG | cathepsin G | cathepsin G |
| CUX1 | Homeobox Protein Cut-Like 1 | cut like homeobox 1 |
| CXCL8 | IL-8, Monocyte-Derived Neutrophil | C-X-C motif chemokine ligand 8 |
| | Chemotactic Factor | |
| CXCL9 | Small-Inducible Cytokine B9 | C-X-C motif chemokine ligand 9 |
| CXCR1 | CD181, interleukin 8 receptor, alpha (IL8RA) | C-X-C motif chemokine receptor 1 |
| CYBB | Neutrophil Cytochrome B 91 KDa | cytochrome b-245 beta chain |
| | Polypeptide, NADPH oxidase 2 | |
| ELANE | Neutrophil Elastase | elastase, neutrophil expressed |
| FCGR1A | CD64, Fc Gamma Receptor Ia | Fc fragment of IgG receptor Ia |
| FCGR3A | CD16a Antigen | Fc fragment of IgG receptor IIIa |
| FCGR3B | CD16b Antigen | Fc fragment of IgG receptor IIIb |
| FES | C-Fes/Fps Protein | FES proto-oncogene, tyrosine kinase |
| FGR | Tyrosine-Protein Kinase Fgr | FGR proto-oncogene, Src family tyrosine |
| | | kinase |
| FPR1 | N-Formylpeptide Chemoattractant Receptor | formyl peptide receptor 1 |
| FPR2 | Lipoxin A4 Receptor | formyl peptide receptor 2 |
| FPR3 | N-Formyl Peptide Receptor 3 | formyl peptide receptor 3 |
| GATA1 | GATA binding protein 1 | GATA binding protein 1 |
| GATA2 | GATA binding protein 2 | GATA binding protein 2 |
| HCK | Hemopoietic Cell Kinase | HCK proto-oncogene, Src family tyrosine |
| | | kinase |
| HOXA10 | homeobox A10 | homeobox A10 |
| IL18 | interleukin 18, interferon-gamma-inducing | interleukin 18 |
| | factor | |
| IL1B | interleukin 1 beta | interleukin 1 beta |
| IL3 | interleukin 3 | interleukin 3 |
| IL6 | interleukin 6 | interleukin 6 |
| ITGAD | CD11d Antigen | integrin subunit alpha D |
| ITGAM | MAC-1, CD11b Antigen | integrin subunit alpha M |
| ITGAX | CD11c Antigen, Myeloid Membrane Antigen, | integrin subunit alpha X |
| | Alpha Subunit | |
| ITGB2 | CD18, macrophage antigen 1 (mac-1) beta | integrin subunit beta 2 |
| | subunit | |
| JUN | C-Jun | Jun proto-oncogene, AP-1 transcription |
| | | factor subunit |
| LTB4R | G Protein-Coupled Receptor 16 | leukotriene B4 receptor |
| LTF | Neutrophil Lactoferrin | lactotransferrin |
| LYZ | lysozyme | lysozyme |
| MMP12 | Macrophage Metalloelastase | matrix metallopeptidase 12 |
| MMP2 | MMP-2, Neutrophil Gelatinase | matrix metallopeptidase 2 |
| MMP8 | MMP-8, Neutrophil Collagenase | matrix metallopeptidase 8 |
| MPEG1 | MPG1, Macrophage-Expressed Gene 1 | macrophage expressed 1 |
| | Protein | |
| MPO | myeloperoxidase | myeloperoxidase |
| MRC1 | CD206, Macrophage Mannose Receptor 1 | mannose receptor C-type 1 |
| MSR1 | CD204 Antigen, Macrophage Scavenger | macrophage scavenger receptor 1 |
| | Receptor Type III | |
| MYB | C-Myb | MYB proto-oncogene, transcription factor |
| MYC | C-Myc, Myc Proto-Oncogene Protein | MYC proto-oncogene, bHLH |
| | | transcription factor |
| MZF1 | MZF-1, Myeloid Zinc Finger 1 | myeloid zinc finger 1 |
| NCF1 | p47phox, Neutrophil NADPH Oxidase Factor 1 | neutrophil cytosolic factor 1 |
| NCF2 | P67PHOX, neutrophil cytosolic factor 2 | neutrophil cytosolic factor 2 |
| PLAU | Urokinase-Type Plasminogen Activator | plasminogen activator, urokinase |
| RUNX1 | RUNX1, Acute Myeloid Leukemia 1 Protein | runt related transcription factor 1 |
| S100A9 | Calgranulin B, Leukocyte L1 Complex Heavy | S100 calcium binding protein A9 |
| | Chain | |
| SATB1 | Special AT-Rich Sequence-Binding Protein 1 | SATB homeobox 1 |
| SERPINA1 | Alpha-1 Protease Inhibitor | serpin family A member 1 |
| SIGLEC1 | CD169 Antigen | sialic acid binding Ig like lectin 1 |
| SLC11A1 | Natural Resistance-Associated Macrophage | solute carrier family 11 member 1 |
| | Protein 1 | |
| SLPI | Antileukoproteinase | secretory leukocyte peptidase inhibitor |
| SP1 | Transcription Factor Sp1 | Sp1 transcription factor |
| SPI1 | PU.1, Transcription Factor PU.1 | Spi-1 proto-oncogene |
| TNF | tumor necrosis factor | tumor necrosis factor |
| TP53 | p53, tumor protein p53 | tumor protein p53 |

### Example 3. LOUP is a 1d-eRNA that Arises from the Upstream Region of the PU.1 Locus

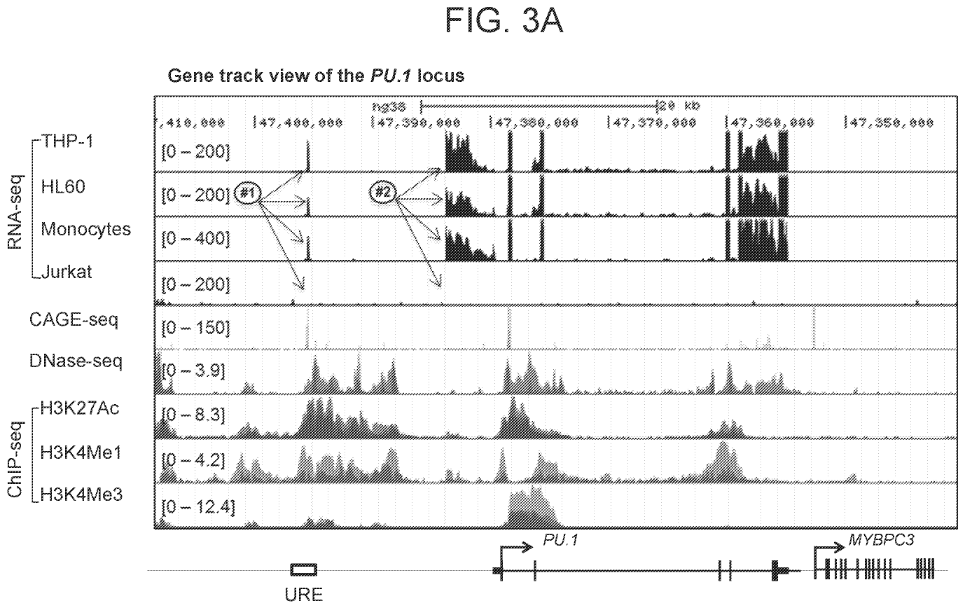

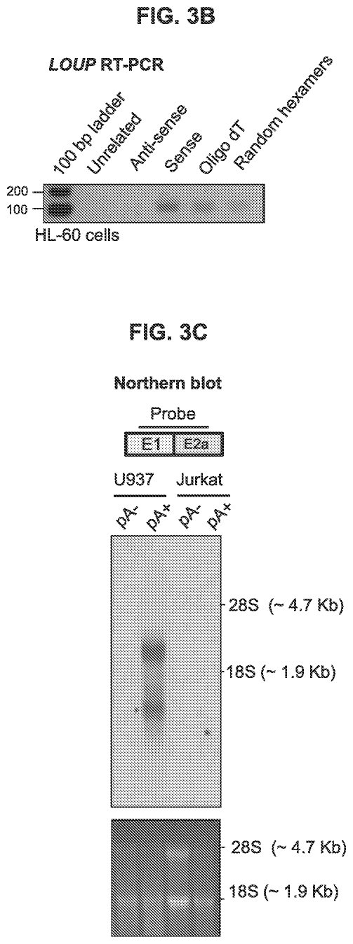

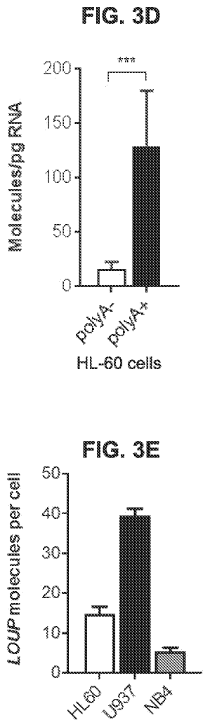

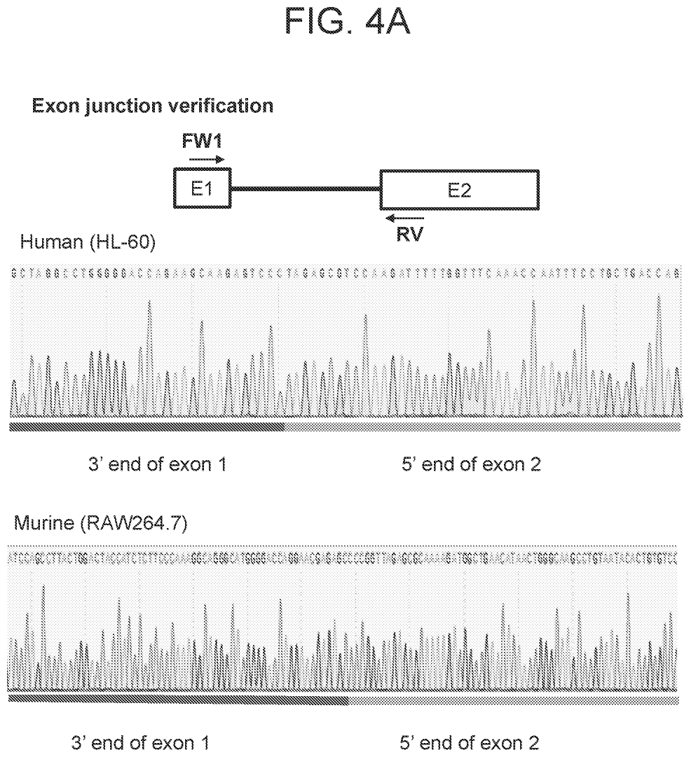

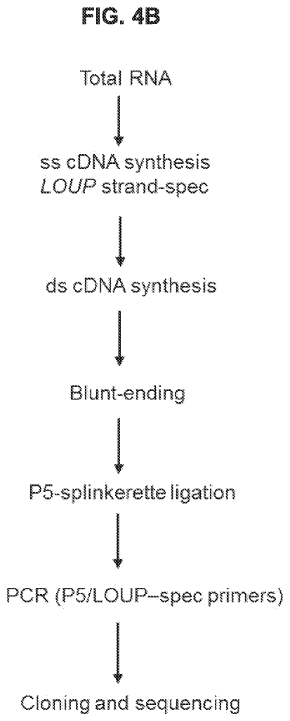

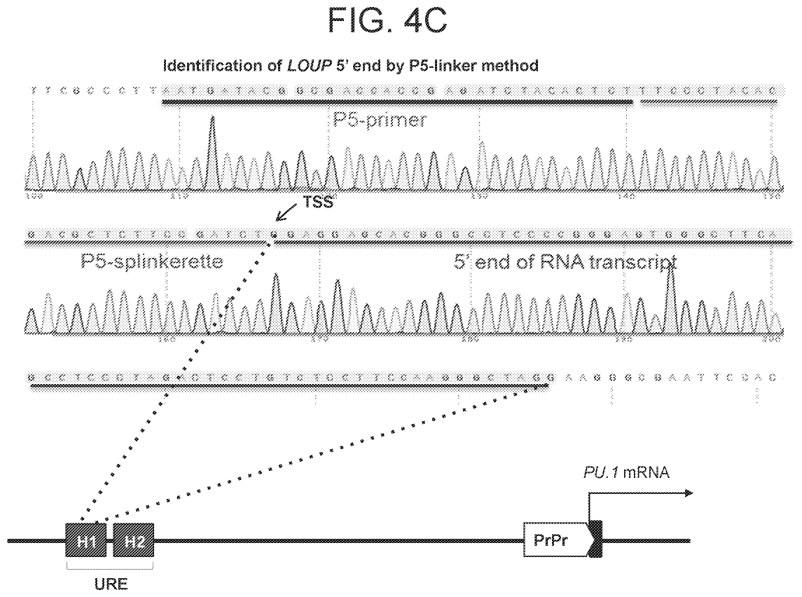

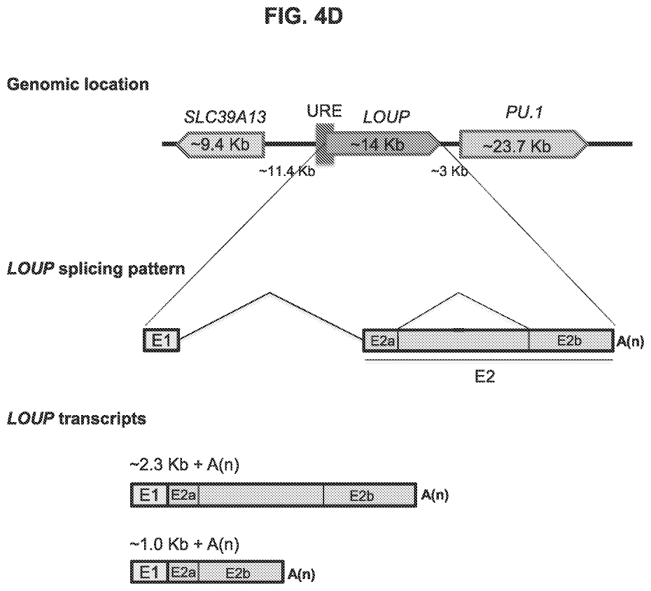

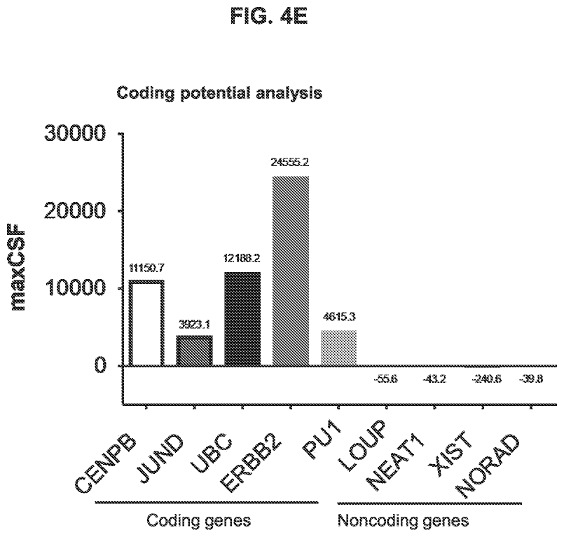

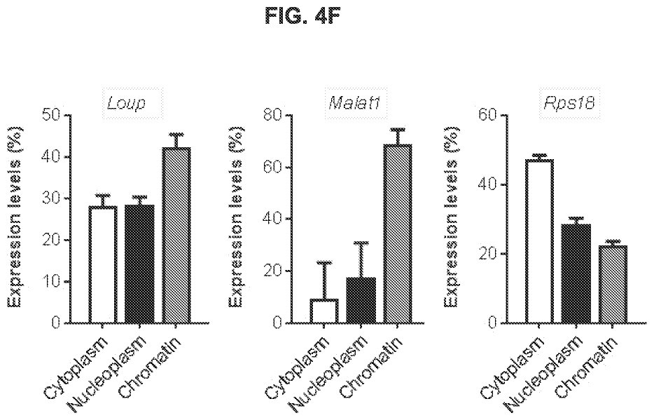

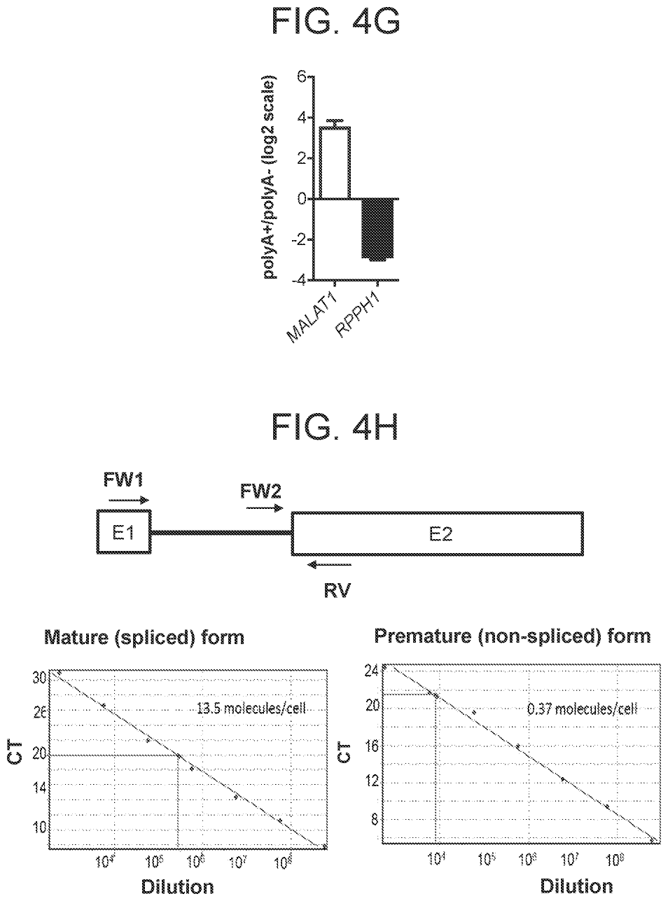

To map the RUNX1-interacting transcript(s), we inspected RNA expression and epigenetic landscapes at the upstream region of the PU.1 locus (FIG. 3A). RNA-seq track view revealed two distinct RNA peaks. A narrow peak was observed at the URE, which corresponded to an area of open chromatin in myeloid cells as indicated by strong DNase I hypersensitivity signals (FIG. 3A, DNase-seq). This element was also enriched with histone post-translational modifications such as H3K27ac, H3K4me1 and H3K4me3 (FIG. 3A, ChIP-seq), which are typical features of active enhancers (Creyghton et al., PNAS 107: 21931-21936, 2010; Pekowska et al., EMBO J. 30: 4198-4210, 2011). A broad peak was proximal to the promoter region. Notably, these peaks were present in myeloid cell lines (THP-1 and HL-60) and primary monocytes but not in the lymphoid cell line Jurkat, indicating a cell-type specific expression pattern. To examine potential connection between these two peaks, we queried genomic region harboring the peaks into the Ensembl browser (Zerbino et al., Nucl. Acid Res. 46:D754-D761, 2018), which contains a comprehensive catalog of verified and predicted RNA transcripts annotated by the HAVANA project, and revealed a predicted human RNA transcript (ENST00000527426.1) with two exons overlapping the observed peaks. Another predicted murine homolog was also described (ENSMUST00000131400.1). RT-PCR and Sanger sequencing analysis confirmed exon junctions in both human and murine cell lines (FIG. 4A). Strand-specific RT-PCR analysis confirmed that the transcript is sense to PU.1 (FIG. 4B). To locate the 5′ end, we inspected Cap analysis gene expression sequencing (CAGE-seq) track from the FANTOM5 project (Kodzius et al., Nat. Methods 3:211-222, 2006) and identified a strong CAGE-seq peak, located within the URE and in the sense genomic orientation (FIG. 4A, CAGE-seq), suggesting the presence of a 5′ transcript end. Using the P5-linker ligation method outlined in FIG. 4B, we identified the 5′ end including a transcription start site (TSS) of the RNA at the homology region 1 (H1) of the URE (Ebralidze et al., Genes Dev. 22: 2085-2092, 2008) (FIG. 4C). Although a splicing event was detected within the second exon, intron retention was dominant as shown by the presence of a ˜2.3 Kb major transcript and a minor ˜1.0 Kb transcript (FIG. 3C and FIG. 4D). The transcripts were detectable in the myeloid cell line U937 but not in the lymphoid cell line Jurkat, further indicating their cell-type specificity (FIG. 3C).

We next determined molecular features of the full-length URE-originating RNA. The RNA exhibited very low coding potential similar to that of other known lncRNAs (FIG. 4E) as assessed by PhyloCSF software (Lin et al., Bioinformatics 27: i275-i282, 2011). Additionally, no known protein domains were found (data not shown) using PFAM software (Finn et al., Nucleic Acids Res. 44: D279-D285, 2016). Thus, we named the RNA transcript “long noncoding RNA originating from the URE of PU.1”, or “LOUP”. Subcellular fractionation, followed by qRT-PCR assays, revealed that LOUP resides in both the cytoplasm and the nucleoplasm compartments, and was particularly enriched in the chromatin fraction (FIG. 4F). The lncRNA is polyadenylated as shown by its detection from total RNA by RT-PCR using Oligo dT primers to generate cDNAs (FIG. 3B) and its robust enrichment in the polyA₊ RNA fraction confirmed by qRT-PCR and Northern blot analyses (FIGS. 3C-3D and FIG. 4G). LOUP is low abundant lncRNA, presenting as its spliced form in ˜14, 40 and 5 copies per cells in HL-60, U937, and NB4, respectively (FIG. 3E). The lncRNA was barely detectable as its premature (non-spliced) form in total RNA as well as in the nuclear RNA fraction (FIGS. 4H-4I). Altogether, these findings established LOUP as a 1d-eRNA that emanates from the URE and extends toward the PrPr.

### Example 4. LOUP is Myeloid-Specific lncRNA that Correlates with PU.1 mRNA Levels

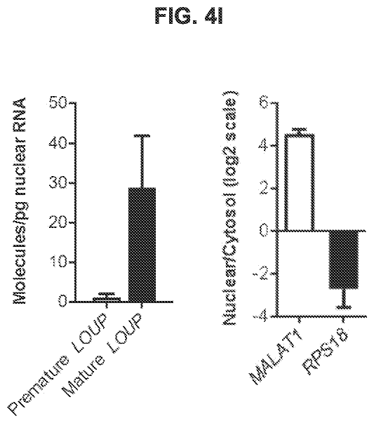

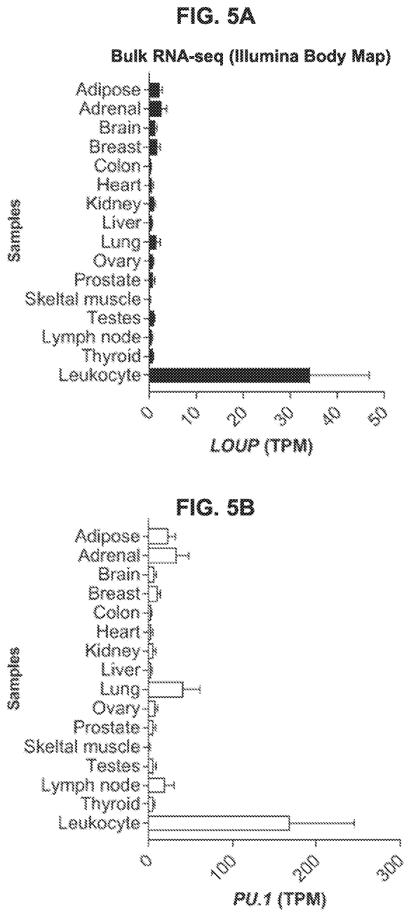

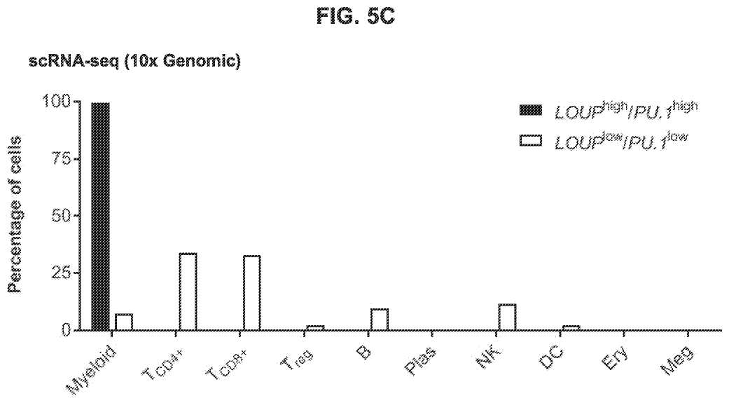

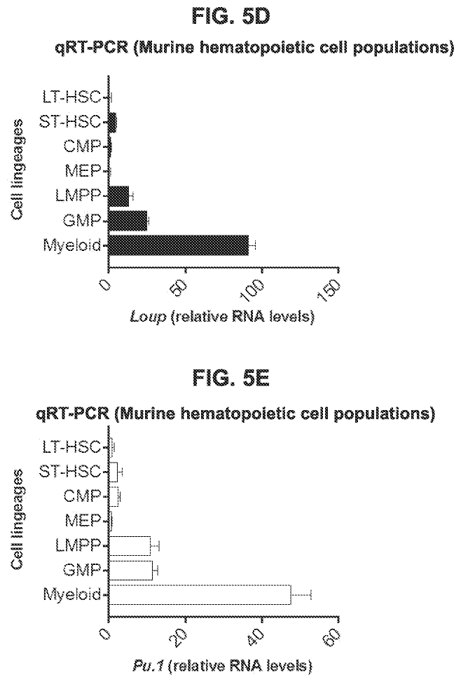

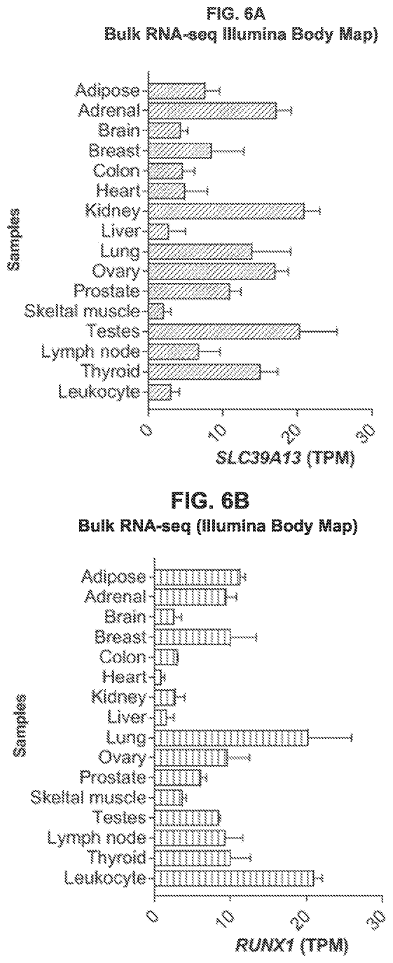

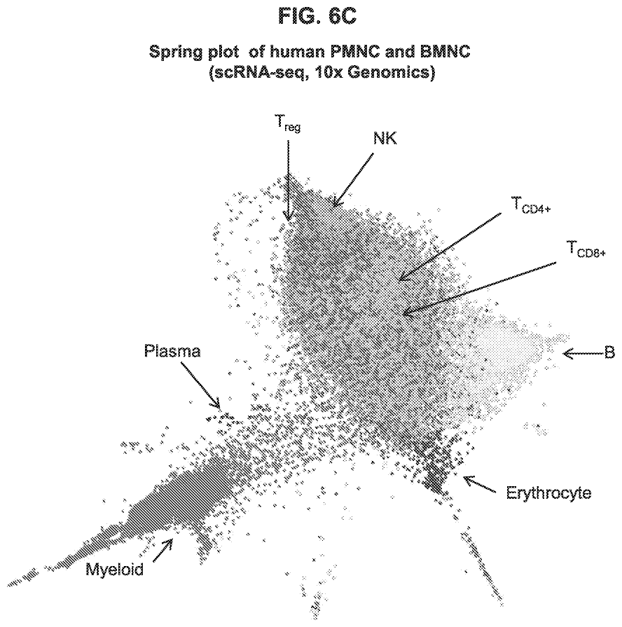

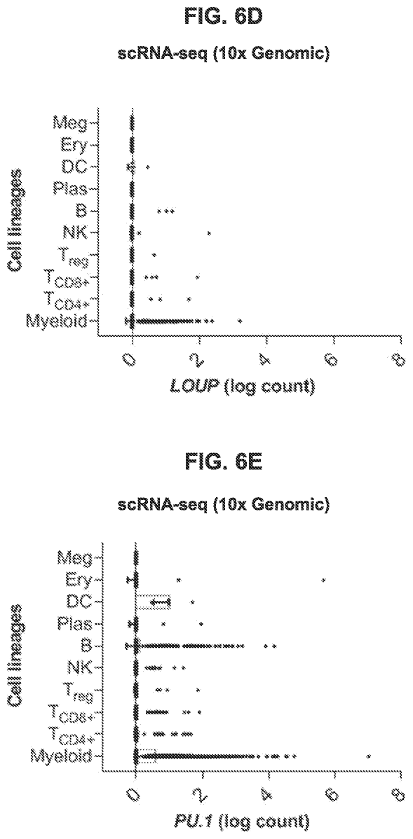

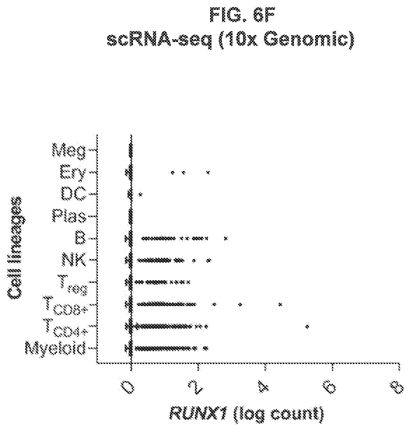

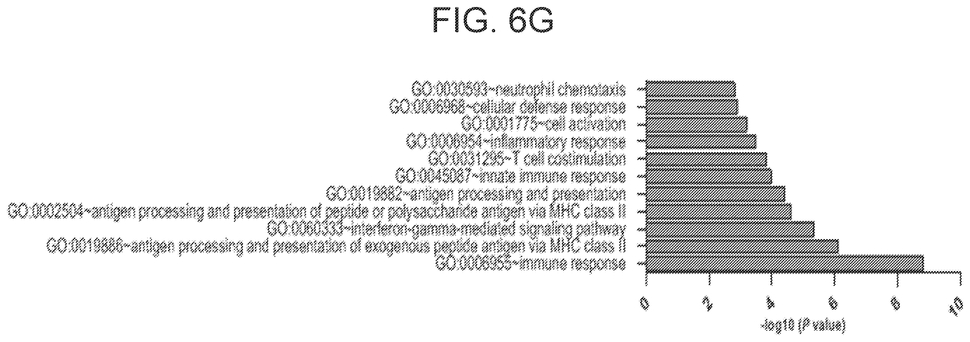

We sought to explore the LOUP expression landscape in normal tissues and cell types. By examining the LOUP transcript profile in different human tissue types from the Illumina Body Map dataset (Illumina), we noticed that this lncRNA was barely detectable in most tissues but elevated in leukocytes (FIG. 5A). Remarkably, comparison with two of its closest neighbor genes, PU.1 and SLC39A13 (FIG. 4D), LOUP expression pattern was similar to that of PU.1 (FIGS. 5A-5B) but not of SLC39A13 (FIG. 6A). Additionally, LOUP transcript levels were not correlated with that of its interacting partner, RUNX1 (FIG. 6B). To further delineate the relationship between LOUP and PU.1 transcript levels in individual blood cells and their lineage identity, we employed single-cell RNA-seq analyses (scRNA-seq). scRNA-seq data of human mononuclear cells isolated from peripheral blood (PBMC) and bone marrow (BMMC) were retrieved from the 10× Genomic Project (Zheng et al., Nat. Commun. 8: 14049, 2017) and pooled together to maximize coverage of hematopoietic cell lineages (FIG. 6C). Notably, LOUP and PU.1 were both enriched in the myeloid cells comprising mono, macrophage and granulocyte (FIGS. 6D-6E). Expectedly, RUNX1 was ubiquitously expressed in myeloid as well as lymphoid cells including T, B, and Natural Killer (NK) (FIG. 6F). By stratifying PBMC and BMMC population into LOUP^{high}/PU.1^{high} and LOUP^{low}/PU.1^{low} groups based on LOUP and PU.1 expression levels (see methods for details), we noted that LOUP^{low}/PU.1^{low} cells were associated with T, B and NK cells. Remarkably, 99.3% of LOUP^{high}/PU.1^{high} cells were associated with myeloid identity (FIG. 5C). Consistent with this observation, top biological processes associated with LOUP and PU.1 expression were mono/macrophage and granulocyte functions (FIG. 5G and Table 5). We further examined LOUP and PU.1 expression pattern during myeloid differentiation. RT-qPCR analyses of purified murine hematopoietic cell populations showed low LOUP levels in long-term hematopoietic stem cells (LT-HSC), short-term hematopoietic stem cells (ST-HSC), common myeloid progenitors (CMP) and megakaryocyte-erythroid progenitors (MEP). Remarkably, the transcript level was elevated in myeloid progenitor cells (granulocyte-macrophage progenitors, GMP) and was highest in definitive myeloid cells (FIG. 5D). A similar expression pattern was seen with PU.1 (FIG. 5E). Taken together, our data indicate that LOUP and PU.1 levels are correlated and associate with myeloid identity, warranting further investigation regarding molecular relationship between LOUP and PU.1 in myeloid cells.

TABLE 5
List of enriched genes in LOUP^{high}/PU.1^{high} cells

| Gene symbol | Gene Name |
|---|---|
| LYPD2 | LY6/PLAUR domain containing 2(LYPD2) |
| SAT1 | spermidine/spermine N1-acetyltransferase 1(SAT1) |
| NEAT1 | nuclear paraspeckle assembly transcript 1 (non-protein coding)(NEAT1) |
| AIF1 | allograft inflammatory factor 1(AIF1) |
| S100A9 | S100 calcium binding protein A9(S100A9) |
| SPI1 | PU.1, Spi-1 proto-oncogene(SPI1) |
| SLC7A7 | solute carrier family 7 member 7(SLC7A7) |
| CFP | complement factor properdin(CFP) |
| WARS | tryptophanyl-tRNA synthetase(WARS) |
| APOBEC3A | apolipoprotein B mRNA editing enzyme catalytic subunit 3A(APOBEC3A) |
| SERPINA1 | serpin family A member 1 (SERPINA1) |
| FCGR3A | Fc fragment of IgG receptor IIIa(FCGR3A) |
| CFD | complement factor D(CFD) |
| PILRA | paired immunoglobin like type 2 receptor alpha(PILRA) |
| FTL | ferritin light chain(FTL) |
| MS4A7 | membrane spanning 4-domains A7(MS4A7) |
| C5AR1 | complement C5a receptor 1(C5AR1) |
| NCF2 | neutrophil cytosolic factor 2(NCF2) |
| LYZ | lysozyme(LYZ) |
| CST3 | cystatin C(CST3) |
| STXBP2 | syntaxin binding protein 2(STXBP2) |
| CTSS | cathepsin S(CTSS) |
| LRRC25 | leucine rich repeat containing 25(LRRC25) |
| IGSF6 | immunoglobulin superfamily member 6(IGSF6) |
| C1QA | complement C1q A chain(C1QA) |
| NPC2 | NPC intracellular cholesterol transporter 2(NPC2) |
| GPBAR1 | G protein-coupled bile acid receptor 1(GPBAR1) |
| HES4 | hes family bHLH transcription factor 4(HES4) |
| GRN | granulin precursor(GRN) |
| MNDA | myeloid cell nuclear differentiation antigen(MNDA) |
| VMO1 | vitelline membrane outer layer 1 homolog(VMO1) |
| LST1 | leukocyte specific transcript 1(LST1) |
| IFITM3 | interferon induced transmembrane protein 3(IFITM3) |
| IFI30 | IF130, lysosomal thiol reductase(IFI30) |
| TYMP | thymidine phosphorylase(TYMP) |
| CD68 | CD68 molecule(CD68) |
| FCN1 | ficolin 1(FCN1) |
| FCER1G | Fc fragment of IgE receptor Ig(FCER1G) |
| FGL2 | fibrinogen like 2(FGL2) |
| SLC31A2 | solute carrier family 31 member 2(SLC31A2) |
| TYROBP | TYRO protein tyrosine kinase binding protein(TYROBP) |
| CEBPB | CCAAT/enhancer binding protein beta(CEBPB) |
| LGALS3 | galectin 3(LGALS3) |
| PSAP | prosaposin(PSAP) |
| LGALS1 | galectin 1(LGALS1) |
| HCK | HCK proto-oncogene, Src family tyrosine kinase(HCK) |
| S100A11 | S100 calcium binding protein A11(S100A11) |
| ANXA5 | annexin A5(ANXA5) |
| COTL1 | coactosin like F-actin binding protein 1(COTL1) |
| CPVL | carboxypeptidase, vitellogenic like(CPVL) |
| ANXA2 | annexin A2(ANXA2) |
| CYBB | cytochrome b-245 beta chain(CYBB) |
| KLF4 | Kruppel like factor 4(KLF4) |

### Example 5. LOUP Acts as a lncRNA Regulator of PU.1 Induction

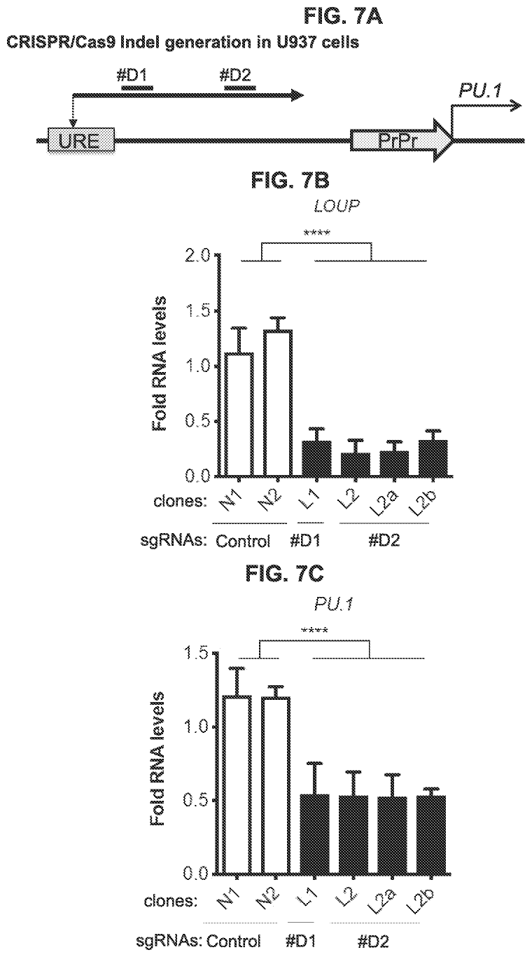

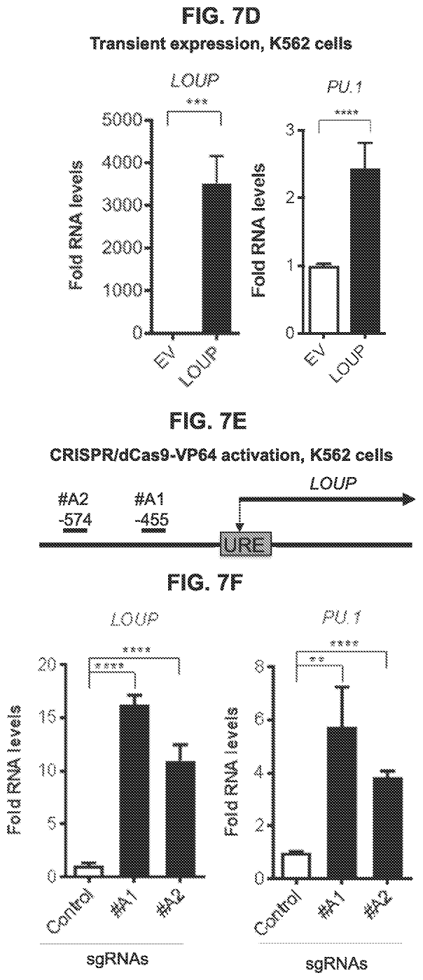

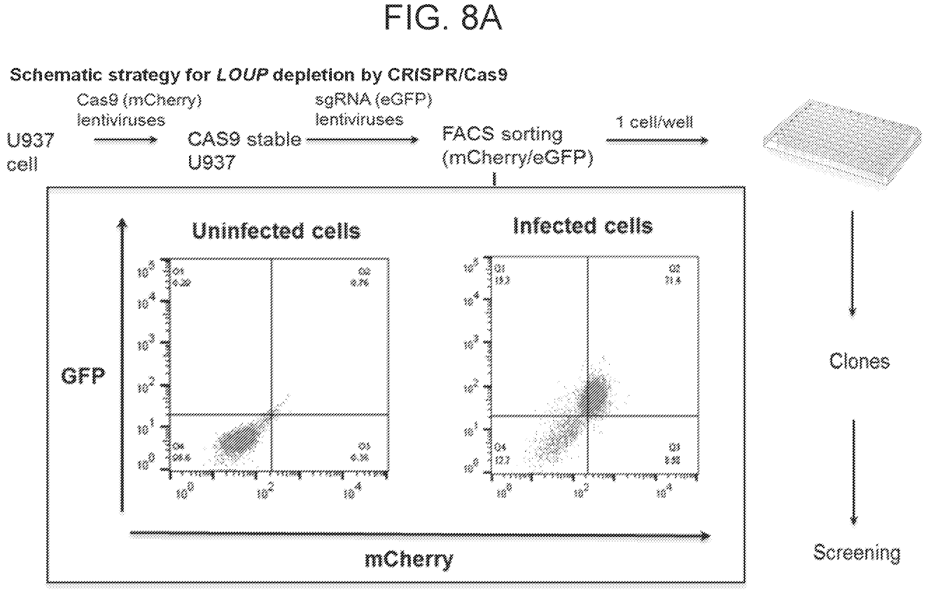

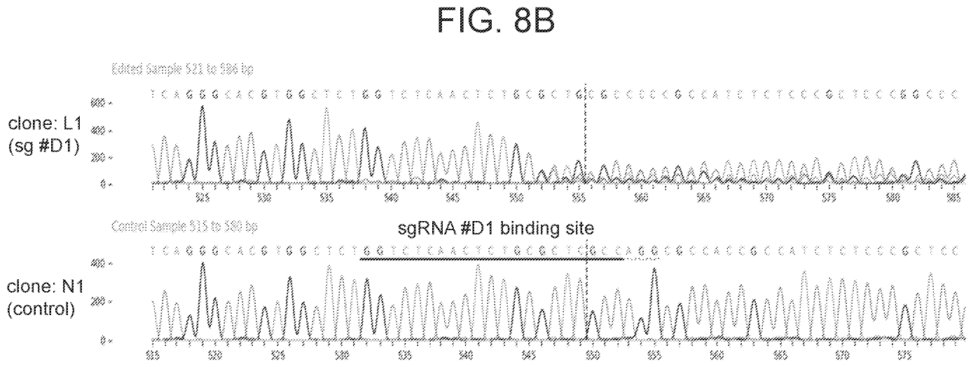

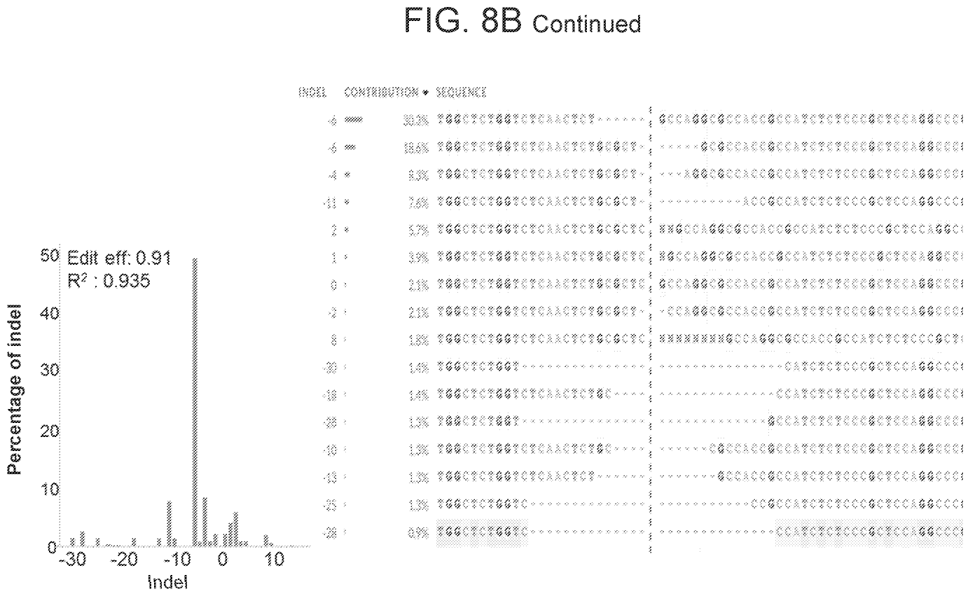

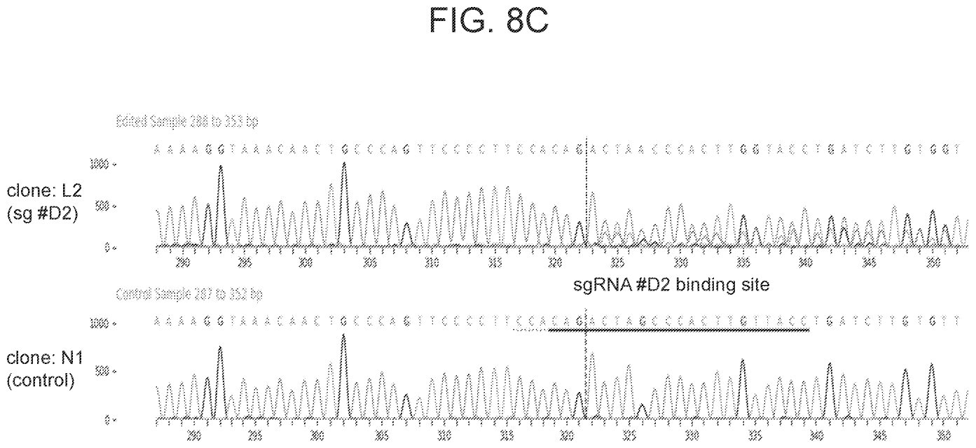

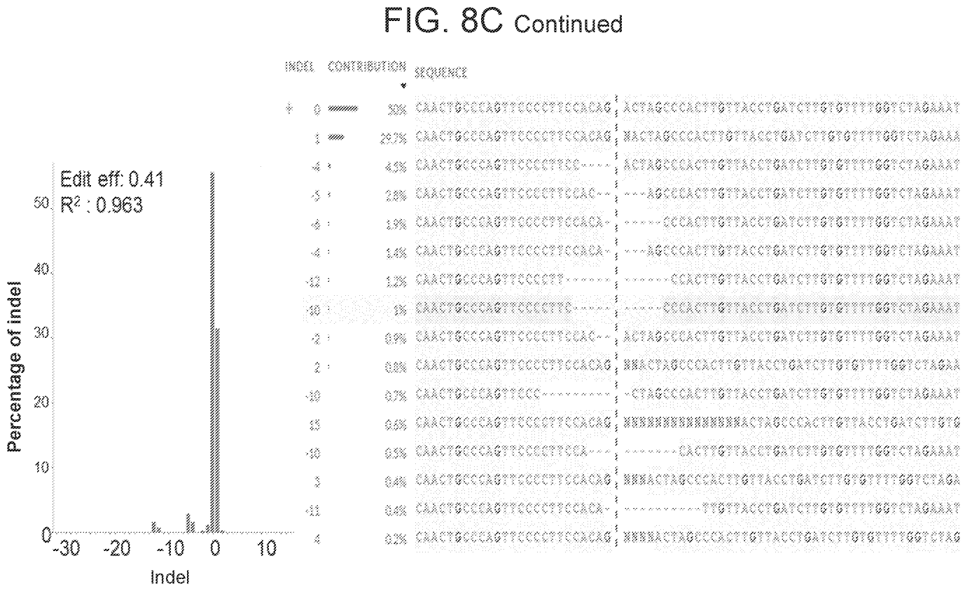

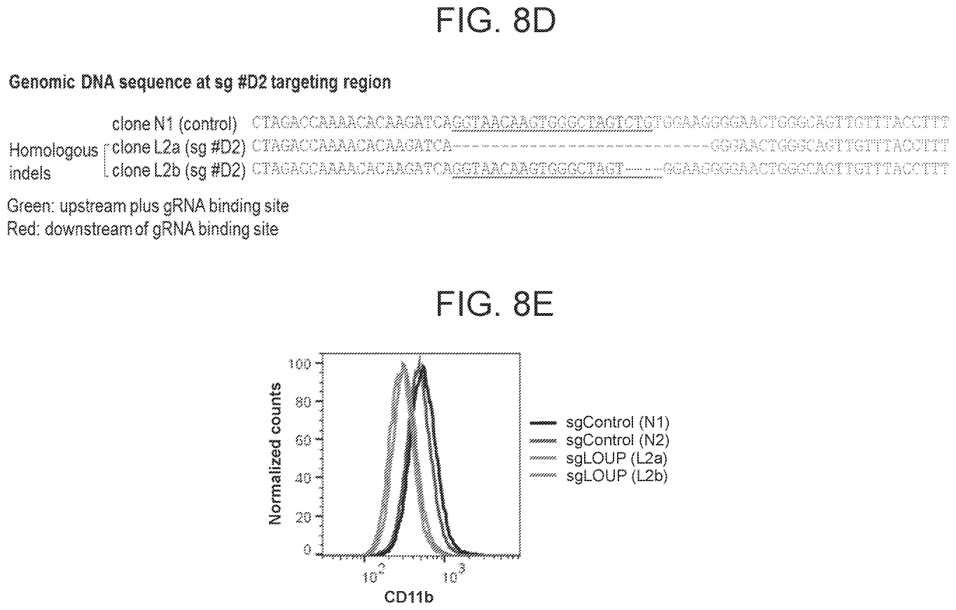

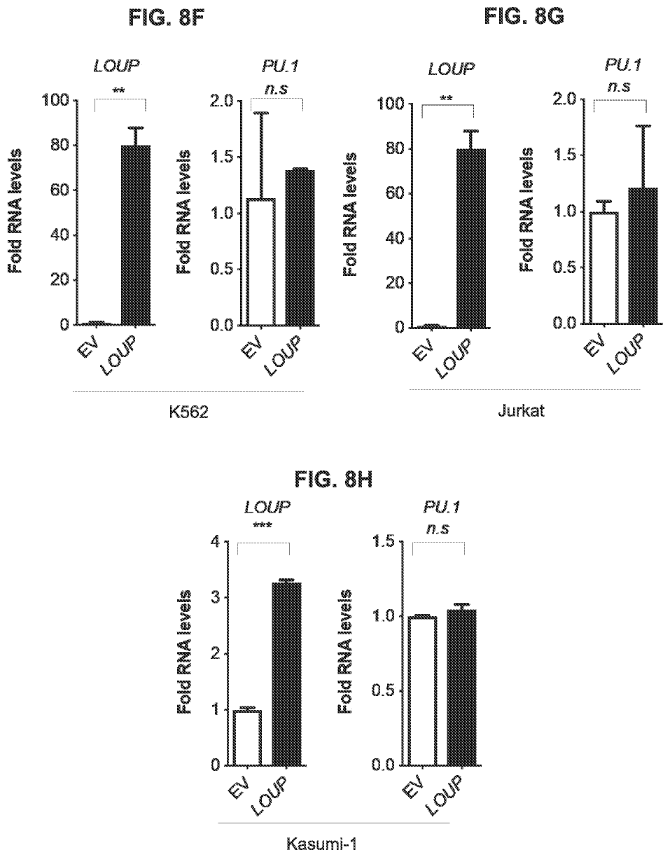

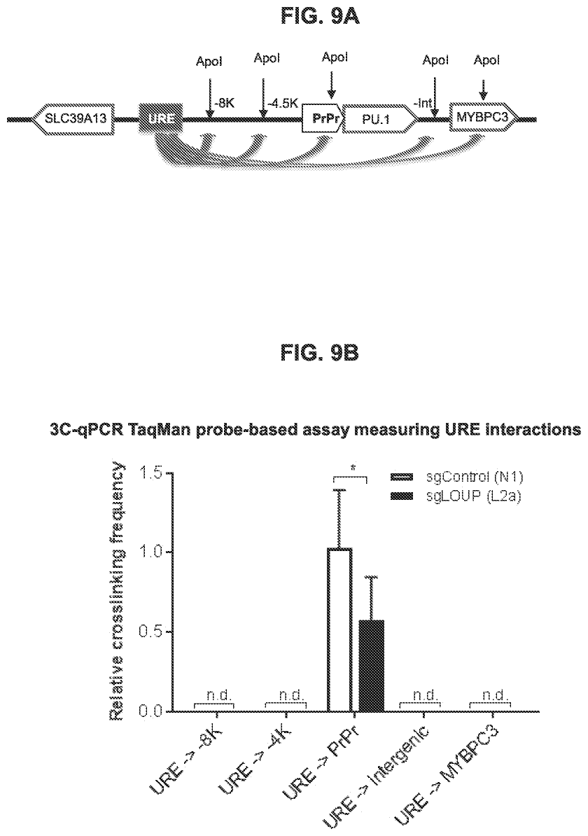

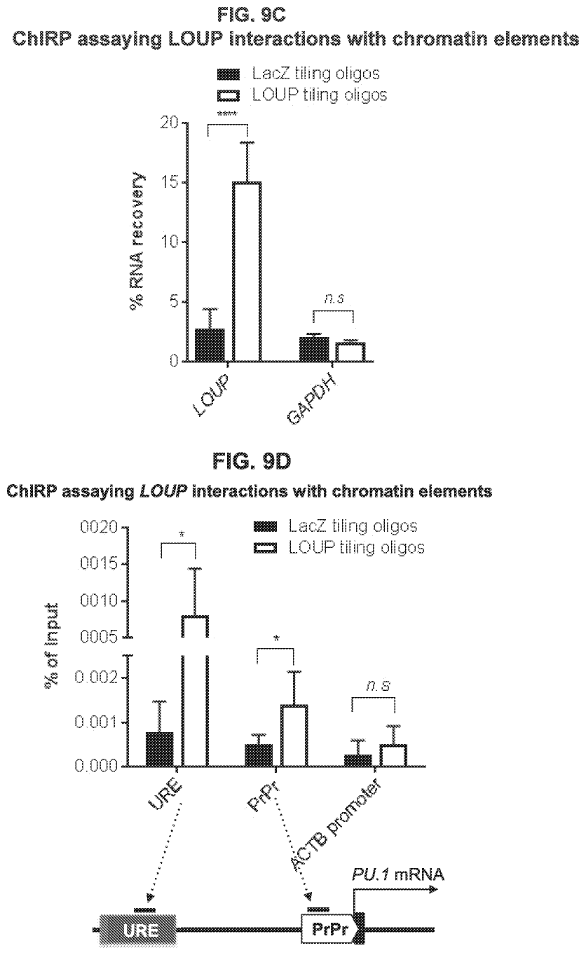

To test our hypothesis that LOUP induces PU.1 expression, we investigated the impact of LOUP's loss-of-expression on PU.1 cellular levels. In order to deplete LOUP RNA transcripts, we employed CRISPR/Cas9 genome-editing technology to introduce small insertion and deletion (indel) mutations in LOUP gene via the non-homologous end-joining (NHEJ) DNA repair mechanism (Jiang et al., Nat. Biotechnol. 31: 233-239 2013; Jinek et al., Science 337: 816-821, 2012). The macrophage cell line U937 that expresses the high level of LOUP (FIG. 3E) was stably transduced with lentiviruses carrying Cas9 and LOUP-targeting or non-targeting sgRNAs. Double-positive mCherry (CAS9) and eGFP (sgRNA) cells were selected by fluorescence-activated cell sorting (FACS) (FIGS. 7A and 8A) and derived cell clones were analyzed by Sanger DNA sequencing and Inference of CRISPR edits (ICE) analysis (Hsiau, et al. BioRxiv251082 2018). LOUP-targeted U937 clones having indels at targeted genomic locations (FIGS. 8B-8D) displayed >80% depletion of LOUP levels which were paralleled by a significant reduction in PU.1 levels (FIGS. 7B-7C). Consistent with the important role of PU.1 in myeloid differentiation (Cook et al., Blood 104: 3437-3444, 2004; Rosenbauer et al., Nat. Genet. 36: 624-630, 2004; Tenen, Nat. Rev. Cancer 3: 89-101, 2003; Walter et al., PNAS 102: 12513-12518, 2005), LOUP depletion associated with a reduction in expression of the myeloid marker CD11b (FIG. 8E).

In converse experiments, transient in trans-overexpression of LOUP in K562 cells resulted in significant induction of PU.1 (FIG. 7D). Remarkably, in cis locus-specific induction of endogenous LOUP via CRISPR/dCas9-VP64 activation system yielded a comparable increase in PU.1 expression as the ectopic in trans-expression, despite producing lower LOUP levels (FIGS. 7E-7F). Inversely, stable ectopic expression of LOUP in K562 and several other cell lines via lentiviral transduction, which integrates randomly into the genome, did not increase PU.1 expression (FIGS. 8F-8H). Together, these results demonstrate that LOUP is a lncRNA regulator of PU.1 and that LOUP exerts its regulatory effect in a cis manner.

### Example 6. LOUP Induces URE-PrPr Communication by Interacting with Chromatin at the PU.1 Locus

We have previously reported that the formation of a chromatin loop mediated by URE-PrPr interaction is crucial for PU.1 induction (Ebralidze et al., 2008, supra; Staber et al., 2013, supra). Because LOUP arises from the URE and extends toward the PrPr, we reasoned that LOUP drives long-range transcription of PU.1 by promoting URE-PrPr interaction. To elucidate this, we quantified the strength of URE interactions with the PrPr and surrounding viewpoints by chromosome conformation capture (3C) followed by qPCR (FIG. 9A). Consistent with previous reports (Ebralidze et al., 2008, supra; Staber et al., 2013, supra), we detected strong interaction of the URE with the PrPr but not with other genomic regions, including the upstream PU.1 promoter, intergenic sequences, and the MYBPC3gene body. Interestingly, reduction in the crosslinking frequency between the URE and the PrPr was observed in LOUP-depleted U937 cells as compared to non-targeting control cells (FIG. 9B). To provide evidence supporting our prediction that LOUP recruits the URE to the PrPr by physically interacting with the two elements, we employed Chromatin Isolation by RNA Purification (ChIRP) assay (Chu et al., 2012, supra). Biotinylated LOUP-tiling oligos were able to capture endogenous LOUP RNA in U937 cells (FIG. 9C). Enrichment of the URE and the PrPr co-captured with LOUP RNA was observed in ChIRPed samples with LOUP-tiling probes but not LacZ-tiling controls, suggesting that LOUP occupies both the URE and the PrPr (FIG. 9D). Taken together, these data indicate that by interacting and bringing to close proximity two regulatory elements, the URE and the PrPr, LOUP promotes the formation of a functional chromatin loop within the PU.1 locus that is critical in inducing PU.1 expression.

### Example 7. LOUP Coordinates Recruitment of RUNX1 to Both the URE and the PrPr

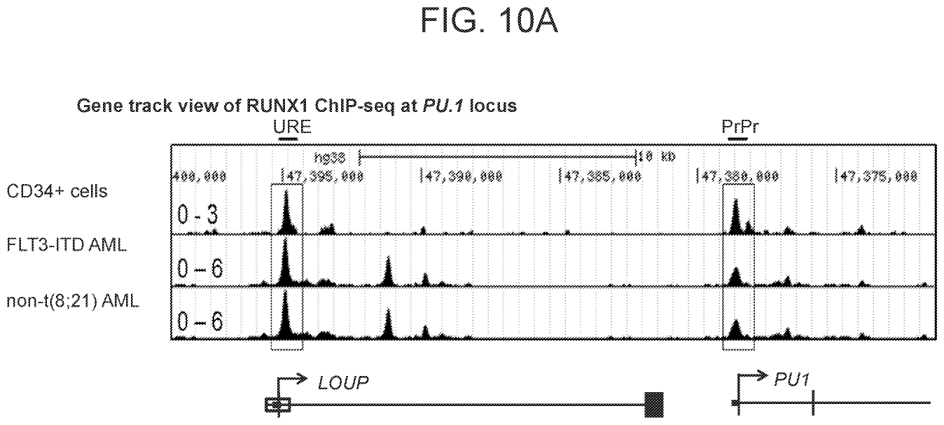

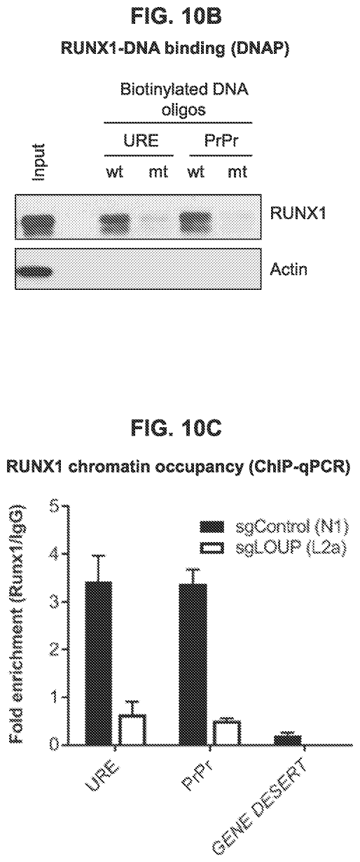

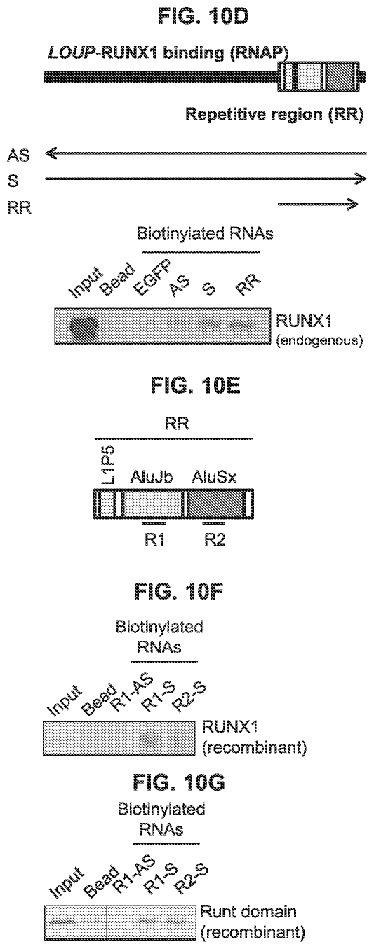

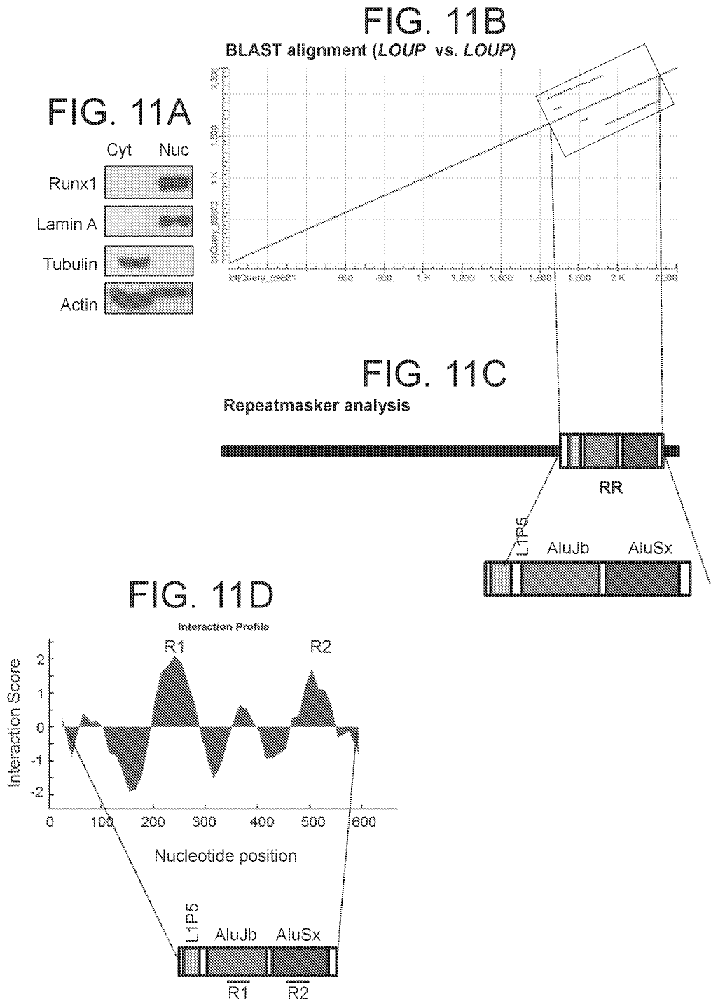

We next sought to gain a deeper mechanistic understanding of how LOUP modulates the chromatin structure in a gene specific manner. Point mutations abrogating the Runx binding sites in the URE are known to disrupt chromatin loop formation (Staber et al., 2014, supra). Additionally, we showed that LOUP interacts with RUNX1 at the PU.1 locus (FIG. 1). Therefore, we asked whether LOUP mediates the URE-PrPr interaction by cooperating with RUNX1. In line with previous finding in murine cells (Staber et al., 2014, id), we observed RUNX1 occupancy at the URE in primary CD34⁺ cells isolated from healthy donor and patients with AML. Importantly, we also noticed a peak at the PrPr, indicating that RUNX1 also occupies the PrPr (FIG. 10A). We further performed biotinylated DNA pull-down (DNAP) assay. Wild-type probes, containing the RUNX consensus motifs embedded in the URE and the PrPr, efficiently captured endogenous RUNX1 from U937 nuclear extract. In contrast, mutant probes lacking the RUNX1 binding sequence, displayed drastic reductions in RUNX1 occupancy (FIG. 10B and FIG. 11A). These results suggest that RUNX1 binds its DNA consensus motif at both the URE and the PrPr. RUNX1 is known to form homodimers to modulate transcription (Bowers et al., Nucleic Acids Res. 38: 6124-6134, 2010; Li et al., J. Biol. Chem. 282: 13542-13551, 2007). Thus, we reasoned that LOUP promotes looping formation by conferring occupancy of RUNX1 dimers concurrently at their binding motifs within the URE and the PrPr. Indeed, LOUP depletion reduced RUNX1 occupancy at both the URE and the PrPr (FIG. 10C), indicating that LOUP promotes placement of RUNX1 dimers at the URE and the PrPr.

### Example 8. LOUP Possesses Embedded TEs that Bind the Runt Domain of RUNX1

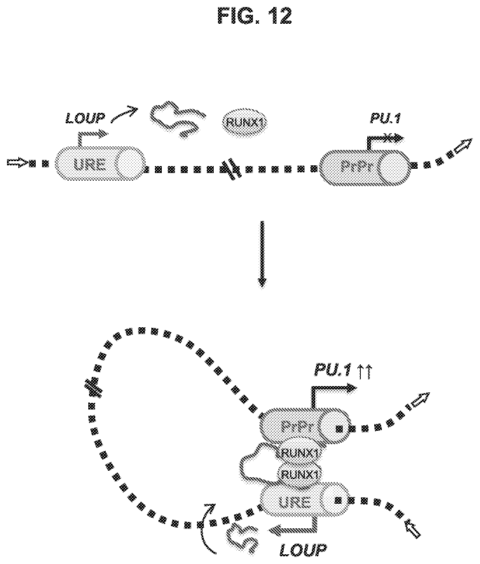

By aligning LOUP sequence with itself using the Basic Local Alignment Search Tool (BLAST), we unexpectedly uncovered a highly repetitive region (RR) of 670 bp near the 3′ end of LOUP (FIG. 11B). We identified, using Repeatmasker analysis, three TE variants clustered in the RR. These include a 3′ end of a LINE-1 retrotransposon variant (L1 PB4) (Howell and Usdin, Mol. Biol. Evol. 14:144-155, 1997; Khan et al., Genome Res. 16: 78-87, 2006) and two Alu SINE variants (AluJb and AluSx) (Price et al., Genome Res. 14: 2245-2252, 2004) (FIG. 11C). Embedded TEs are implicated to serve as functional domains of lncRNAs (Johnson and Guigo, RNA 20: 959-976 2014; Kannan et al., Front. Bioeng. Biotechnol. 3: 71, 2015; Kim et al., RNA 22: 254-264, 2016; Podbevsek et al., Sci. Rep. 8: 3189, 2018). To explore the possibility that these TEs function as a RUNX1-interacting platform for LOUP in the nucleus, we performed RNA pull-down assay (RNAP). Biotinylated LOUP RR was able to capture endogenous RUNX1 proteins in U937 nuclear extract at a level that is comparable to biotinylated full-length LOUP, indicating that the RR contains RUNX1-binding region (FIG. 10D). To locate the region, we first computed potential interaction strength of putative elements within the RR to RUNX1 protein by using catRAPID algorithm (Bellucci et al., Nat. Methods 8: 444-445, 2011). By doing so, we identified two ˜100 bp candidate regions, termed region 1 (R1) and R2, within two Alu variants with high interaction scores (FIG. 11D and FIG. 10E). RNAP analysis confirmed that R1 and R2 bind to recombinant RUNX1 (FIG. 10F). Additionally, recombinant Runt domain of RUNX1 was able to bind R1 and R2 (FIG. 10G) suggesting that the domain is responsible for LOUP binding. These data, together, demonstrate that LOUP binds RUNX1 and coordinates deposition of RUNX1 dimers to the URE and the PrPr (FIG. 12).

### Example 9. Diagnosis of a Disease or Disorder in a Subject

A subject can be diagnosed as having a disease or disorder associated with PU.1 expression (e.g., a cancer (e.g., AML, liver cancer, or myeloma), Alzheimer's disease, or asthma) as described herein. The diagnostic method can be performed by determining a level of the transcription factor PU. 1 in a subject or a level of LOUP expression in a subject as described herein.

For example, a sample (e.g., a tissue sample, a blood sample, a cell sample, or a fluidic sample) can be obtained from a subject (e.g., a subject suspected of having a disease or disorder) and analyzed for LOUP and/or PU.1 expression. The level of LOUP and/or PU.1 expression can be compared to a standard or reference level (e.g., a control sample, in which a known expression level of LOUP and/or PU.1 has been linked to the presence or absence of the disease or disorder) or to a sample from a reference subject (e.g., a subject known to be healthy (e.g., to lack the disease or disorder) or a subject known to have the disease or disorder). Comparison of the LOUP and/or PU.1 level to the standard or reference level can confirm the presence or absence of the disease or disorder in the subject being tested.

For example, a subject determined to have decreased expression of PU.1, as compared to a standard or reference, can be identified as having or at risk of developing a cancer (e.g., AML, liver cancer, or myeloma). Alternatively, a subject determined to have increased expression of PU.1, as compared to a standard or reference, can be identified as having or at risk of developing Alzheimer's disease or asthma.

For example, a subject determined to have decreased expression of LOUP, as compared to a standard or reference, can be identified as having or at risk of developing a cancer (e.g., AML, liver cancer, or myeloma). Alternatively, a subject determined to have increased expression of LOUP, as compared to a standard or reference, can be identified as having or at risk of developing Alzheimer's disease or asthma.

Gene sequencing methods (e.g., next-generation gene sequencing methods, e.g., high-throughput sequencing, including but not limited to, Illumina sequencing, Roche 454 sequencing, Ion torrent: Proton/PGM sequencing, and SOLiD sequencing) can be used to analyze PU.1 and/or LOUP expression for the diagnosis of a disease or disorder.

### Example 10. Diagnosing a subject as susceptible to ATRA treatment

Also provided are methods of diagnosing a subject as having a cancer (e.g., AML) that is susceptible to differentiation therapy with all-trans retinoic acid (ATRA) based on LOUP expression. A sample (e.g., a tissue sample, a blood sample, a cell sample, or a fluidic sample) from a subject (e.g., a subject suspected of having a cancer) can be analyzed for LOUP expression and compared to a standard or reference level (e.g., a control sample, in which a known expression level of LOUP has been linked to the presence or absence of the disease or disorder) or to a sample from a reference subject (e.g., a subject known to be healthy (e.g., to lack the disease or disorder) or a subject known to have the disease or disorder). Comparison of the LOUP level to the standard or reference level can be used to determine if the subject is likely to be sensitive to differentiation therapy with ATRA. For example, low levels of LOUP (relative to a standard or reference) would indicate resistance of the cancer to ATRA therapy.

Gene sequencing methods (e.g., next-generation gene sequencing methods, e.g., high-throughput sequencing, including but not limited to, Illumina sequencing, Roche 454 sequencing, Ion torrent: Proton/PGM sequencing, and SOLiD sequencing) can be used to analyze PU.1 and/or LOUP expression for the diagnosis of a disease or disorder.

### Example 11. Gene Editing Systems for Targeting LOUP Expression

A gene editing system, as described herein, can be used to target LOUP expression in a subject (e.g., a subject in need thereof) for the treatment of a PU.1 associated medical condition. As an example, a gene editing system can be designed to be directed to a target genomic site associated with LOUP (e.g., a LOUP transcription start site or the LOUP gene).

After identifying a target genomic site, deep gene sequencing methods can be used to identify suitable PAM sites to be used for targeting of the gene editing system. Methods of designing the sgRNA are described herein. A delivery vehicle can be developed that includes the CRISPR/Cas nuclease (e.g., an active CRISPR/Cas nuclease or a CRISPRa gene activating system) and the sgRNA that can be used to direct the CRISPR/Cas nuclease to the target genomic site of interest. Non-limiting examples of LOUP targeting are described below.

For treating a disease associated with decreased PU.1 expression (e.g., a cancer (e.g., AML, liver cancer, or myeloma)) a CRISPRa gene activating system can be designed to increase LOUP expression. Briefly, sgRNAs targeting the upstream region of LOUP's transcriptional start site can be designed using Cas-Designer (Park et al., 2015, supra). As described above, the CRISPRa gene activating system (e.g., a dCas9-VP64) can be incorporated into a delivery vehicle (e.g., a vector (e.g., a viral vector (e.g., a lentiviral vector))) along with the sgRNA, and, optionally, one or more promoters to induce expression of the gene editing system. The delivery vehicle can be administered to a subject in need thereof (e.g., a subject having a disease or disorder associated with a decreased PU.1 expression (e.g., a cancer (e.g., AML, liver cancer, or myeloma))) and provide the gene editing system to a target cell for LOUP activation.

Alternatively, for treating a disease associated with increase PU.1 expression (e.g., Alzheimer's disease or Asthma) it may be beneficial to decrease PU.1 expression by decreasing LOUP expression (e.g., “knocking out” LOUP). Briefly, LOUP-targeting sgRNAs can be designed as described herein using Cas-Designer (Park et al., Bioinformatics 31: 4014-4016, 2015). To avoid disruption of the URE, known to be critical for PU.1 induction (Li et al. Blood 98: 2958-2965, 2001), single-guide RNAs (sgRNA) targeting LOUP (e.g., two distinct regions of the LOUP gene: (1) the LOUP intronic area downstream of the URE, and (2) the intronic area right upstream of the second exon of the LOUP gene (˜15 kb downstream from the URE)) can be designed and cloned into a delivery vehicle (e.g., a vector (e.g., a lentiviral vector) also incorporating the CRISPR/Cas system. The delivery vehicle can be formulated for administration to a subject in need thereof (e.g., a subject having a disease or disorder associated with an increased PU.1 expression (e.g., Alzheimer's or asthma)) and provide the gene editing system to a target cell for LOUP knock out.

### Example 12. Treating a Disease or Disorder Associated with Decreased PU.1 Expression

A subject in need of treatment for a disease or disorder associated identified as having reduced expression of the transcription factor PU.1 (e.g., a cancer, such as AML, liver cancer, or myeloma), as described herein, can be administered a composition including a featured polynucleotide that increases expression of PU.1.

For treatment of a disease or disorder associated with reduced expression of PU.1, generally, a composition containing the featured polynucleotide (e.g., a polynucleotide including at least 20 nucleotides of SEQ ID NO: 1) can be administered (e.g., intravenously) to a subject (e.g., a subject in need thereof, such as a human) as a medicament (e.g., for treating a medical condition (e.g., a cancer (e.g., a PU.1 associated cancer (e.g., AML, liver cancer, or myeloma)))). The featured polynucleotide described herein can be used to induce the expression of tumor suppressor gene PU.1, thereby treating the disease or disorder. The featured polynucleotide can be delivered as a vector (e.g., a viral vector or non-viral vector) described herein. In certain embodiments, the featured polynucleotide can be delivered as a vector including a nucleic acid encoding the featured polynucleotide (e.g., a polynucleotide including at least 20 nucleotides of SEQ ID NO: 1) as described herein. In some embodiments, the vector is a viral vector (e.g., a lentiviral vector or an AAV vector). Gene sequencing methods (e.g., next-generation gene sequencing methods, e.g., high-throughput sequencing, including but not limited to, Illumina sequencing, Roche 454 sequencing, Ion torrent: Proton/PGM sequencing, and SOLiD sequencing) can be used to identify a subject in need thereof (e.g., a subject with a PU.1 associated cancer (e.g., AML, liver cancer, or myeloma)).

### Example 13. Altering PU.1 Expression in a Subject in Need Thereof

The featured long non-coding RNA (e.g., LOUP RNA), polynucleotides encoding the lncRNA (e.g., a polynucleotide having at least 20 nucleotides of SEQ ID NO: 1), vectors (e.g., viral vectors) including polynucleotides encoding the lncRNA, constructs including the lncRNA (e.g., constructs including a protein linked to a LOUP polynucleotide), gene editing system (e.g., a CRISPR/Cas system or CRISPRa) for regulating PU.1 expression, polynucleotides encoding the gene editing systems, and vectors (e.g., viral vectors) including polynucleotides encoding the gene editing system can be administered to a subject in need thereof (e.g., a human) to alter (e.g., increase or decrease) the expression of tumor associated gene PU.1. Compositions and methods for delivering the featured polynucleotides (e.g., a polynucleotide having at least 20 nucleotides of SEQ ID NO: 1) and/or CRISPR/Cas system components include, e.g., a vector (e.g., a viral vector, such as a lentiviral vector particle), and non-vector delivery vehicles (e.g., nanoparticles), as discussed above.

Generally, the methods can include administering a composition containing the polynucleotide (e.g., a polynucleotide including at least 20 nucleotides of SEQ ID NO: 1), a construct thereof, or the gene editing system (e.g., a CRISPR/Cas system CRISPRa), either incorporated as a nucleic acid molecule (e.g., in a vector, such as a viral vector) encoding the polynucleotide, construct, or the components of the gene editing system (e.g., Cas protein and guide polynucleotides (e.g., guide RNA)) to a subject in need thereof. Alternatively, the methods can include administering the gene editing system in protein form (e.g., as a composition containing a Cas protein in combination with one or more guide polynucleotide(s) (e.g., gRNA(s))). The compositions can be administered (e.g., intravenously or intracranially) to a subject (e.g., a subject in need thereof) as a medicament for the treatment of a medical condition associated with PU.1 expression.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. All publications, patents, and patent applications mentioned in the above specification are hereby incorporated by reference to the same extent as if each individual publication, patent or patent application was specifically and individually indicated to be incorporated by reference in its entirety.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Other embodiments are within the claims.

1. A polynucleotide comprising a sequence with at least 20 nucleotides of SEQ ID NO: 1, and variants thereof with at least 85% sequence identity thereto, wherein the polynucleotide has fewer than 2,381 nucleotides of SEQ ID NO: 1.

2. The polynucleotide of claim 1, wherein the variant of the polynucleotide has at least 90%, 95%, 97%, or 100% sequence identity to SEQ ID NO: 1.

3. The polynucleotide of claim 1 or 2, wherein the polynucleotide comprises a binding region for a Runt-related transcription factor 1 (RUNX1) protein or fragment thereof.

4. The polynucleotide of claim 3, wherein the binding region comprises all or at least 20 nucleotides of one or more transposable elements (TEs).

5. The polynucleotide of claim 4, wherein the one or more TEs comprise a nucleotide sequence with at least 85% sequence identity to at least 20 or more nucleotides of any one of SEQ ID NOs: 2-4.

6. The polynucleotide of claim 5, wherein the polynucleotide comprises two said TEs or three said TEs.

7. The polynucleotide of claim 6, wherein the polynucleotide comprises three said TEs, and wherein a first said TE comprises at least 20 nucleotides of SEQ ID NO: 2, a second said TE comprises at least 20 nucleotides of SEQ ID NO: 3, and a third said TE comprises at least 20 nucleotides of SEQ ID NO: 4.

8. The polynucleotide of claim 7, wherein the three said TEs comprise SEQ ID NOs: 2-4.

9. The polynucleotide of claim 7 or 8, wherein the first, second, and third TEs are present in the polynucleotide in order, 5′ to 3′, and wherein the TEs are linked directly or through a linker.

10. The polynucleotide of any one of claims 1-9, wherein the polynucleotide comprises at least 30 nucleotides of SEQ ID NO: 1.

11. The polynucleotide of any one of claims 1-10, wherein the polynucleotide comprises at least 40 nucleotides of SEQ ID NO: 1.

12. The polynucleotide of any one of claims 1-11, wherein the polynucleotide comprises at least 100 nucleotides of SEQ ID NO: 1.

13. The polynucleotide of any one of claims 1-12, wherein the polynucleotide comprises at least 500 nucleotides of SEQ ID NO: 1.

14. The polynucleotide of any one of claims 1-13, wherein the polynucleotide comprises at least 1700 nucleotides of SEQ ID NO: 1.

15. The polynucleotide of any one of claims 1-14, wherein the polynucleotide comprises at least 2000 nucleotides of SEQ ID NO: 1.

16. The polynucleotide of any one of claims 1-15, wherein the polynucleotide comprises at least 2300 nucleotides of SEQ ID NO: 1.

17. The polynucleotide of any one of claims 1-16, wherein the polynucleotide comprises at least 2350 nucleotides of SEQ ID NO: 1.

18. The polynucleotide of any one of claims 1-17, wherein the polynucleotide comprises at least 2375 nucleotides of SEQ ID NO: 1.

19. A construct comprising a RUNX1 protein, or fragment thereof, conjugated to at least one polynucleotide of any one of claims 1-18.

20. The construct of claim 19, wherein the construct comprises at least one said RUNX1 protein, or fragment thereof, bound to at least one said polynucleotide.

21. The construct of claim 19 or 20, wherein the RUNX1 protein, or fragment thereof, and the polynucleotide are bound through a covalent bond.

22. The construct of any one of claims 19-21, comprising the structure:
R-L-P (I) or P-L-R (II),
wherein R is the RUNX1 protein or fragment thereof;
P is the polynucleotide; and
L is a linker.

23. The construct of claim 22, where the construct comprises the structure of R-L-P (I).

24. The construct of claim 22, wherein the construct comprises the structure of P-L-R (II).

25. The construct of any one of claims 22-24, wherein R comprises at least 100 amino acids of SEQ ID NO: 5, and variants thereof with at least 85% sequence identity thereto.

26. The construct of claim 25, wherein R has at least 90%, 95%, 97%, or 100% sequence identity to the sequence of SEQ ID NO: 5.

27. The construct of claim 26, wherein R polypeptide has the sequence of SEQ ID NO: 5.

28. The construct of any one of claims 22-27, wherein R polypeptide comprises at least one binding site for at least one polynucleotide regulatory element of PU.1.

29. The construct of claim 28, wherein the at least one PU.1 regulatory element has at least 85% sequence identity to the sequence of SEQ ID NO: 6.

30. The construct of claim 29, wherein the at least one PU.1 regulatory element has at least 90%, 95%, 97%, or 100% sequence identity to the sequence of SEQ ID NO: 6.

31. The construct of claim 30, wherein the at least one PU.1 regulatory element has the sequence of SEQ ID NO: 6.

32. The construct of claim 28, wherein the at least one PU.1 regulatory element is an upstream regulatory element (URE) and/or a proximal promoter region (PrPr).

33. The construct of claim 32, wherein the PrPr has at least 85% sequence identity to the sequence of SEQ ID NO: 7.

34. The construct of claim 33, wherein the PrPr has at least 90%, 95%, 97%, or 100% sequence identity to the sequence of SEQ ID NO: 7.

35. The construct of claim 34, wherein the PrPr has the sequence of SEQ ID NO: 7.

36. A polynucleotide encoding the construct of any one of claims 19-35.

37. A vector comprising the polynucleotide of any one of claims 1-18 or the polynucleotide of claim 36.

38. A composition comprising the polynucleotide of any one of claims 1-18, the construct of any one of claims 19-35, the polynucleotide of claim 36, or the vector of claim 37.

39. The composition of claim 38, further comprising a pharmaceutically acceptable carrier, excipient, or diluent.

40. A kit comprising the polynucleotide of any one of claims 1-18, the construct of any one of claims 19-35, the polynucleotide of claim 36, the vector of claim 37, or the composition of claim 38 or 39, and a package insert comprising instructions for using the polynucleotide, construct, vector, or composition for treating a medical condition in a subject.

41. A method of treating a medical condition in a subject in need thereof comprising administering the polynucleotide of any one of claims 1-18.

42. The method of claim 41, wherein the medical condition is a cancer.

43. The method of claim 42, wherein the cancer is a blood cancer.

44. The method of claim 43, wherein the blood cancer is acute myeloid leukemia (AML).

45. The method of claim 43, wherein the blood cancer is myeloma.

46. The method of claim 42, wherein the cancer is liver cancer.

47. The method of claim 46, wherein the liver cancer is metastatic hepatocellular carcinoma (HCC).

48. A method of treating a medical condition in a subject in need thereof comprising administering the construct of any one of claims 19-35.

49. The method of claim 48, wherein the medical condition is a cancer.

50. The method of claim 49, wherein the cancer is a blood cancer.

51. The method of claim 50, wherein the blood cancer is acute myeloid leukemia (AML).

52. The method of claim 50, wherein the blood cancer is myeloma.

53. The method of claim 49, wherein the cancer is liver cancer.

54. The method of claim 53, wherein the liver cancer is metastatic hepatocellular carcinoma (HCC).

55. Use of the construct of any one of claims 19-35 in the preparation of a medicament for the treatment of a medical condition in a subject in need thereof.

56. A method of treating a medical condition in a subject, wherein the method comprises:
a) delivering to a target cell a dCas activator system comprising:
i) a plurality of first guide ribonucleic acids (gRNAs) directed to a first genomic site of an endogenous DNA molecule of the cell; and
ii) a plurality of dCas fusion proteins;
wherein the first gRNA forms a first complex with a first said dCas fusion protein at the first genomic site, and wherein the first complex promotes the expression of LOUP.

57. The method of claim 56, wherein the first guide gRNA specifically hybridizes to the first genomic site.

58. The method of claim 56 or 57, wherein the first genomic site and the target gene of interest are between 10-100,000 nucleotide base pairs apart.

59. The method of any one of claims 56-58, wherein the first genomic site comprises a protospacer adjacent motif (PAM) recognition sequence positioned upstream from said first genomic site.

60. The method of any one of claims 56-59, wherein the first guide RNA is a single guide RNA (sgRNA).

61. The method of any one of claims 56-60, wherein the dCas fusion protein is selected from a group comprising dCas9-VP64, dCas9-VPR, dCas9-SAM, dCas9-Scaffold, dCas9-Suntag, dCas9-P300, dCas9-VP160, and VP64-dCas9-BFP-VP64.

62. The method of claim 61, wherein the dCas fusion protein is dCas9-VP64.

63. The method of any one of claims 56-62, wherein the first target genomic site is associated with the medical condition.

64. The method of any one of claims 56-63, wherein the medical condition is a cancer.

65. The method of claim 64, wherein the cancer is a cancer associated with tumor suppressor gene PU.1.

66. The method of claim 65, wherein the cancer associated with tumor suppressor gene PU.1 is acute myeloid leukemia (AML), liver cancer, or myeloma.

67. The method of any one of claims 56-66, wherein the target gene of interest is tumor suppressor gene PU.1.

68. A nucleic acid comprising a polynucleotide comprising a nucleic acid sequence encoding dCas activator system.

69. The nucleic acid of claim 68, wherein the dCas activator system comprises a dCas fusion protein.

70. The nucleic acid of claim 68 or 69, further comprising a nucleic acid sequence encoding a first gRNA.

71. The nucleic acid of claim 70, wherein the first gRNA is directed to a first genomic site of an endogenous DNA molecule of a cell.

72. The nucleic acid of any one of claims 68-71, further comprising a promoter.

73. The nucleic acid of any one of claims 69-72, wherein the dCas fusion protein is selected from a group comprising dCas9-VP64, dCas9-VPR, dCas9-SAM, dCas9-Scaffold, dCas9-Suntag, dCas9-P300, dCas9-VP160, and VP64-dCas9-BFP-VP64.

74. A vector comprising the nucleic acid of any one of claims 68-73.

75. The vector of claim 74, wherein the vector is an expression vector or a viral vector.

76. The vector of claim 75, wherein the viral vector is a lentiviral vector.

77. A composition comprising:
a) a plurality of first guide ribonucleic acids (gRNAs) directed to a first genomic site of an endogenous DNA molecule of the cell; and
b) a plurality of dCas fusion proteins.

78. The composition of claim 77, wherein the first gRNA is in a first complex with a first said dCas fusion protein,
wherein the first complex is configured to promote the expression of a target gene of interest.

79. The composition of claim 77 or 78, the dCas fusion protein is selected from a group comprising dCas9-VP64, dCas9-VPR, dCas9-SAM, dCas9-Scaffold, dCas9-Suntag, dCas9-P300, dCas9-VP160, and VP64-dCas9-BFP-VP64.

80. The composition of claim 79, wherein the dCas fusion protein is dCas9-VP64.

81. A pharmaceutical composition comprising the nucleic acid of any one of claims 68-76, or the composition of any one of claims 77-79, and a pharmaceutically acceptable carrier, excipient, or diluent.

82. A kit comprising the nucleic acid of any one of claims 68-76, the composition of any one of claims 77-79, or the pharmaceutical composition of claim 81, and a package insert comprising instructions for using the nucleic acid, composition, or pharmaceutical composition for treating a medical condition in a subject.

83. A method of treating a medical condition in a subject, wherein the method comprises:
a) delivering to a target cell a gene editing system comprising:
i) a plurality of first guide ribonucleic acids (gRNAs) directed to a first genomic site of an endogenous DNA molecule of the cell; and
ii) a plurality of RNA programmable nucleases;
wherein the first guide RNA forms a first complex with a first said RNA programmable nuclease at the first genomic site, and wherein the first complex promotes the inhibition of expression of LOUP.

84. The method of claim 83, wherein the first guide gRNA specifically hybridizes to the first genomic site.

85. The method of claim 83 or 84, wherein the first genomic site and the target gene of interest are between 10-100,000 nucleotide base pairs apart.

86. The method of any one of claims 83-85, wherein the first genomic site comprises a protospacer adjacent motif (PAM) recognition sequence positioned upstream from said first genomic site.

87. The method of any one of claims 83-86, wherein the first guide RNA is a single guide RNA (sgRNA).

88. The method of any one of claims 83-87, wherein the inhibition of expression of the target gene of interest is caused by non-homologous end-joining (NHEJ).

89. The method of any one of claims 83-88, wherein the first target genomic site is associated with the medical condition.

90. The method of any one of claims 83-89, wherein the medical condition is associated with tumor suppressor gene PU.1.

91. The method of claim 90, wherein the medical condition associated with PU.1 is Alzheimer's disease or asthma.

92. The method of any one of claims 83-91, wherein the target gene of interest is tumor suppressor gene PU.1.

93. The method of any one of claims 83-92, wherein the RNA program nuclease is a Cas RNA programmable nuclease.

94. The method of claim 93, wherein the Cas RNA programmable nuclease is a Cas9 RNA programmable nuclease.

95. A nucleic acid comprising a polynucleotide comprising a nucleic acid sequence encoding:
a) a first gRNA directed to a first genomic site of an endogenous DNA molecule of a target cell; and
b) an RNA-programmable nuclease;
wherein the first genomic site is between 10-100,000 nucleotide base pairs from a target gene of interest comprising tumor suppressor gene PU.1.

96. The nucleic acid of claim 95, further comprising a promoter.

97. The nucleic acid molecule of claim 95 or 96, wherein the RNA programmable nuclease is a Cas RNA programmable nuclease.

98. The nucleic acid of claim 97, wherein the Cas RNA programmable nuclease is a Cas9 RNA programmable nuclease.

99. A vector comprising the nucleic acid of any one of claims 95-98.

100. The vector of claim 99, wherein the vector is an expression vector or a viral vector.

101. The vector of claim 100, wherein the viral vector is a lentiviral vector.

102. The polynucleotide of claim 1, wherein the polynucleotide comprises a binding region for a RUNX1 protein or fragment thereof.

103. The polynucleotide of claim 102, wherein the binding region comprises all or at least 20 nucleotides of one or more TEs.

104. The polynucleotide of claim 103, wherein the one or more TEs comprise a nucleotide sequence with at least 85% sequence identity to at least 20 or more nucleotides of any one of SEQ ID NOs: 2-4.

105. The polynucleotide of claim 104, wherein the polynucleotide comprises two said TEs or three said TEs.

106. The polynucleotide of claim 105, wherein the polynucleotide comprises three said TEs, and wherein a first said TE comprises at least 20 nucleotides of SEQ ID NO: 2, a second said TE comprises at least 20 nucleotides of SEQ ID NO: 3, and a third said TE comprises at least 20 nucleotides of SEQ ID NO: 4.

107. The polynucleotide of claim 106, wherein the three said TEs comprise SEQ ID NOs: 2-4.

108. The polynucleotide of claim 106, wherein the first, second, and third TEs are present in the polynucleotide in order, 5′ to 3′, and wherein the TEs are linked directly or through a linker.

109. The polynucleotide of claim 1, wherein the polynucleotide comprises at least 30 nucleotides of SEQ ID NO: 1.

110. The polynucleotide of claim 1, wherein the polynucleotide comprises at least 40 nucleotides of SEQ ID NO: 1.

111. The polynucleotide of claim 1, wherein the polynucleotide comprises at least 100 nucleotides of SEQ ID NO: 1.

112. The polynucleotide of claim 1, wherein the polynucleotide comprises at least 500 nucleotides of SEQ ID NO: 1.

113. The polynucleotide of claim 1, wherein the polynucleotide comprises at least 1700 nucleotides of SEQ ID NO: 1.

114. The polynucleotide of claim 1, wherein the polynucleotide comprises at least 2000 nucleotides of SEQ ID NO: 1.

115. The polynucleotide of claim 1, wherein the polynucleotide comprises at least 2300 nucleotides of SEQ ID NO: 1.

116. The polynucleotide of claim 1, wherein the polynucleotide comprises at least 2350 nucleotides of SEQ ID NO: 1.

117. The polynucleotide of claim 1, wherein the polynucleotide comprises at least 2375 nucleotides of SEQ ID NO: 1.

118. A construct comprising a RUNX1 protein, or fragment thereof, conjugated to at least one polynucleotide of claim 1.

119. The construct of claim 118, wherein the construct comprises at least one said RUNX1 protein, or fragment thereof, bound to at least one said polynucleotide.

120. The construct of claim 118, wherein the RUNX1 protein, or fragment thereof, and the polynucleotide are bound through a covalent bond.

121. The construct of claim 118, comprising the structure:
R-L-P (I) or P-L-R (II),
wherein R is the RUNX1 protein or fragment thereof;
P is the polynucleotide; and
L is a linker.

122. The construct of claim 121, where the construct comprises the structure of R-L-P (I).

123. The construct of claim 121, wherein the construct comprises the structure of P-L-R (II).

124. The construct of claim 121, wherein R comprises at least 100 amino acids of SEQ ID NO: 5, and variants thereof with at least 85% sequence identity thereto.

125. The construct of claim 124, wherein R has at least 90%, 95%, 97%, or 100% sequence identity to the sequence of SEQ ID NO: 5.

126. The construct of claim 125, wherein R polypeptide has the sequence of SEQ ID NO: 5.

127. The construct of claim 121, wherein R polypeptide comprises at least one binding site for at least one polynucleotide regulatory element of PU.1.

128. The construct of claim 127, wherein the at least one PU.1 regulatory element has at least 85% sequence identity to the sequence of SEQ ID NO: 6.

129. The construct of claim 128, wherein the at least one PU.1 regulatory element has at least 90%, 95%, 97%, or 100% sequence identity to the sequence of SEQ ID NO: 6.

130. The construct of claim 129, wherein the at least one PU.1 regulatory element has the sequence of SEQ ID NO: 6.

131. The construct of claim 127, wherein the at least one PU.1 regulatory element is an upstream regulatory element (URE) and/or a proximal promoter region (PrPr).

132. The construct of claim 131, wherein the PrPr has at least 85% sequence identity to the sequence of SEQ ID NO: 7.

133. The construct of claim 132, wherein the PrPr has at least 90%, 95%, 97%, or 100% sequence identity to the sequence of SEQ ID NO: 7.

134. The construct of claim 133, wherein the PrPr has the sequence of SEQ ID NO: 7.

135. A polynucleotide encoding the construct of claim 118.

136. A vector comprising the polynucleotide of claim 1.

137. A composition comprising the polynucleotide of claim 1, a construct comprising a RUNX1 protein, or fragment thereof, conjugated to the polynucleotide, a polynucleotide encoding the construct, or a vector comprising the polynucleotide of claim 1.

138. The composition of claim 137, further comprising a pharmaceutically acceptable carrier, excipient, or diluent.

139. A kit comprising the polynucleotide of claim 1, a construct comprising a RUNX1 protein, or fragment thereof, conjugated to the polynucleotide, a polynucleotide encoding the construct, a vector comprising the polynucleotide of claim 1, or a composition comprising the polynucleotide of claim 1, and a package insert comprising instructions for using the polynucleotide, construct, vector, or composition for treating a medical condition in a subject.

140. A method of treating a medical condition in a subject in need thereof comprising administering the polynucleotide of claim 1.

141. The method of claim 140, wherein the medical condition is a cancer.

142. The method of claim 141, wherein the cancer is a blood cancer.

143. The method of claim 142, wherein the blood cancer is acute myeloid leukemia (AML).

144. The method of claim 142, wherein the blood cancer is myeloma.

145. The method of claim 141, wherein the cancer is liver cancer.

146. The method of claim 145, wherein the liver cancer is metastatic hepatocellular carcinoma (HCC).

147. A method of treating a medical condition in a subject in need thereof comprising administering the construct of claim 118.

148. The method of claim 147, wherein the medical condition is a cancer.

149. The method of claim 148, wherein the cancer is a blood cancer.

150. The method of claim 149, wherein the blood cancer is acute myeloid leukemia (AML).

151. The method of claim 149, wherein the blood cancer is myeloma.

152. The method of claim 148, wherein the cancer is liver cancer.

153. The method of claim 152, wherein the liver cancer is metastatic hepatocellular carcinoma (HCC).

154. Use of the construct of claim 118 in the preparation of a medicament for the treatment of a medical condition in a subject in need thereof.

155. The method of claim 56, wherein the first genomic site and the target gene of interest are between 10-100,000 nucleotide base pairs apart.

156. The method of claim 56, wherein the first genomic site comprises a protospacer adjacent motif (PAM) recognition sequence positioned upstream from said first genomic site.

157. The method of claim 56, wherein the first guide RNA is a single guide RNA (sgRNA).

158. The method of claim 56, wherein the dCas fusion protein is selected from a group comprising dCas9-VP64, dCas9-VPR, dCas9-SAM, dCas9-Scaffold, dCas9-Suntag, dCas9-P300, dCas9-VP160, and VP64-dCas9-BFP-VP64.

159. The method of claim 158, wherein the dCas fusion protein is dCas9-VP64.

160. The method of claim 56, wherein the first target genomic site is associated with the medical condition.

161. The method of claim 56, wherein the medical condition is a cancer.

162. The method of claim 161, wherein the cancer is a cancer associated with tumor suppressor gene PU.1.

163. The method of claim 162, wherein the cancer associated with tumor suppressor gene PU.1 is acute myeloid leukemia (AML), liver cancer, or myeloma.

164. The method of claim 56, wherein the target gene of interest is tumor suppressor gene PU.1.

165. The nucleic acid of claim 68, further comprising a nucleic acid sequence encoding a first gRNA.

166. The nucleic acid of claim 165, wherein the first gRNA is directed to a first genomic site of an endogenous DNA molecule of a cell.

167. The nucleic acid of claim 68, further comprising a promoter.

168. The nucleic acid of claim 69, wherein the dCas fusion protein is selected from a group comprising dCas9-VP64, dCas9-VPR, dCas9-SAM, dCas9-Scaffold, dCas9-Suntag, dCas9-P300, dCas9-VP160, and VP64-dCas9-BFP-VP64.

169. A vector comprising the nucleic acid of claim 68.

170. The vector of claim 169, wherein the vector is an expression vector or a viral vector.

171. The vector of claim 170, wherein the viral vector is a lentiviral vector.

172. The composition of claim 77, the dCas fusion protein is selected from a group comprising dCas9-VP64, dCas9-VPR, dCas9-SAM, dCas9-Scaffold, dCas9-Suntag, dCas9-P300, dCas9-VP160, and VP64-dCas9-BFP-VP64.

173. The composition of claim 79, wherein the dCas fusion protein is dCas9-VP64.

174. A pharmaceutical composition comprising the nucleic acid of claim 68, or a composition comprising (a) a plurality of first gRNAs directed to a first genomic site of an endogenous DNA molecule of the cell and (b) a plurality of dCas fusion proteins, and a pharmaceutically acceptable carrier, excipient, or diluent.

175. A kit comprising the nucleic acid of claim 68, a composition comprising (a) a plurality of first gRNAs directed to a first genomic site of an endogenous DNA molecule of the cell and (b) a plurality of dCas fusion proteins, or a pharmaceutical composition comprising the nucleic acid, and a package insert comprising instructions for using the nucleic acid, composition, or pharmaceutical composition for treating a medical condition in a subject.

176. The method of claim 83, wherein the first genomic site and the target gene of interest are between 10-100,000 nucleotide base pairs apart.

177. The method of claim 83, wherein the first genomic site comprises a protospacer adjacent motif (PAM) recognition sequence positioned upstream from said first genomic site.

178. The method of claim 83, wherein the first guide RNA is a single guide RNA (sgRNA).

179. The method of claim 83, wherein the inhibition of expression of the target gene of interest is caused by non-homologous end-joining (NHEJ).

180. The method of claim 83, wherein the first target genomic site is associated with the medical condition.

181. The method of claim 83, wherein the medical condition is associated with tumor suppressor gene PU.1.

182. The method of claim 181, wherein the medical condition associated with PU.1 is Alzheimer's disease or asthma.

183. The method of claim 83, wherein the target gene of interest is tumor suppressor gene PU.1.

184. The method of claim 83, wherein the RNA program nuclease is a Cas RNA programmable nuclease.

185. The method of claim 184, wherein the Cas RNA programmable nuclease is a Cas9 RNA programmable nuclease.

186. The nucleic acid of claim 95, wherein the RNA programmable nuclease is a Cas RNA programmable nuclease.

187. The nucleic acid of claim 186, wherein the Cas RNA programmable nuclease is a Cas9 RNA programmable nuclease.

188. A vector comprising the nucleic acid of claim 95.

189. The vector of claim 188, wherein the vector is an expression vector or a viral vector.

190. The vector of claim 189, wherein the viral vector is a lentiviral vector.


SEQUENCE LISTING (132 sequences)

SEQ ID NO: 1 - DNA, 2381 bp, Homo sapiens
tggaggagcacgggcctccccgggagtgggcttcagcctccctagactcctgtctccttccaagggctaggcctgggggaccagaagcaagagtccctagagcgtccaagatttttggtttcaaaccaatttcctgctgaccagaatgaaatggagccacattccagcacgatgcacctgctgacctcctgcaagtcagggaccttctctgcccctcttttgctctgctgcaggagggagaagctgggaatgtgttcctctggggtcccatcacctccagccagccactgtccctgtctccccaaaacctcctcgttgtccctcctctgtcctcctcctcctccatctgccttcgccctgctctcctctttttcgcctgaggttcgtgctgcctcctcacctcttgtcctgtttctcccttctctctgctccctcctttgatcttgctattttgcttctctttcccttcctctcttctcctccctccccttcctccctgctccctgcctcagcttctcagtctctctccgttcttcctctagcttccttccctccccagaagacccagcccttcctagagccccagaagccagggttcatgccccagctctctacccacacatgctgtgcaaccctgggccagtcactggtctcttgggcctcagttcaacagatggctgtcttggtgatgtgagatcatagggtggaagcacccagaatggtgccaggcactcaaaaatttttttcttttcctttttaaggaactactaaccgtgaggttatggattaccaaaccgttcctgttcttggaatctgggtcctactattcccaagaatttgaattaccacgggtccctggggatgtaaggggatgaggcttctgatgtactttgggagggggaggagtggaaagaggagttgggaggacactctagcattgcccagagggcagaggacattctgcctatttccagaggttattgaccctggatctcttttatggagttcctcacccaggccccttgagcttggccatacttgaggcaatgggatatcttccttctcctggagccccttgaatcttaggcctcgggaacacacagtctactgtgtttcttggctgcacccacggaggacagcccccatactccttgcttacaaccagcaaagactccacagccatccctgcacacgactcaggctccttggcttggcactcaaggctctttgctttcctgtcctgctttgcttccagctgcaaagatggtggcttcctgttttcctcttcgtttcctttgcttaagttgattctcagccagacatgctttccccctgcacgcttccctaagccatcctatctgggaccctgacccaatcaatggttccctaatgcacctttgagaccaagtgtacctggcacctcctctcttgccccactatctgctcacatgaccctctgcaggcctgtaagctgctggaggctccgagtatgcctcattcatgtctatcccccttactaggcccagaccatggaagacactcaataaaaagtaagtgattaaatgacaacctctacggaaaagagtatggagatttctcaaagaactaaaaacagaactaccttttggccagatgcagtggctcacacctataatcccaacatttgggaaggctgaggcaggtggattgcttgagcccaggagttcaagatcagcctgggcaacatggtgaaaccctatctctacaaaaaatacagaaaattagatgagactacaggtggcacacatccatagtaccagctactccagaggctgaggcgggaggatcgcttgagcccaggaggtccaggctacagtgggccaagatcacgccattgcactccagcctcagtgataaagtggcattctgtctcaaaagaaaaagaactggccaagcatggtggctcacgtctgtaatcccagcactttgggaggccgaggcaggtggatcacatgagatcaggagtttgagactagcctggccaacaatgatgaaaccccatctctactaaaaatacaataattagctgggcgtggtggcgggcgcctgtaatcccagctactcaggaggctgaggcaggagaatcatttgaacccgggaggcaaagtgagccaagattgtgccactgcactccagcctgggcaacaagagcgaaactcagtctcaaaagaaaaagaaaaagagagaaaagaagaaaggaaaagaaatatcatttgatccagcaatctcactgagtatctaaccaaaggaaaataaatcactataccaaaaagaaaaaaaaaaaaaaaaaaaaaaaaaaaaaaaaaaaaaaaaaaaaaaaaaaaaaaaaaaaaaaaaaaaaa

SEQ ID NO: 2 - DNA, 204 bp, Homo sapiens
acaacctctacggaaaagagtatggagatttctcaaagaactaaaaacagaactaccttttggccagatgcagtggctcacacctataatcccaacatttgggaaggctgaggcaggtggattgcttgagcccaggagttcaagatcagcctgggcaacatggtgaaaccctatctctacaaaaaatacagaaaattagatgag

SEQ ID NO: 3 - DNA, 171 bp, Homo sapiens
ctcagtgataaagtggcattctgtctcaaaagaaaaagaactggccaagcatggtggctcacgtctgtaatcccagcactttgggaggccgaggcaggtggatcacatgagatcaggagtttgagactagcctggccaacaatgatgaaaccccatctctactaaaaatac

SEQ ID NO: 4 - DNA, 35 bp, Homo sapiens
gagccaagattgtgccactgcactccagcctgggc

SEQ ID NO: 5 - protein, 480 aa, Homo sapiens
MASDSIFESFPSYPQCFMRECILGMNPSRDVHDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 6 - DNA, 986 bp, Homo sapiens
agagatctgggtactggccagggaggcaggaggaaggagggaaggccgcggggcacccgggggctgctgcctggcaggtccccatgcccaggcaagggaagtttgttatttctcttgcttcgacttccccctttgatttattatagccatgaaatgctctgctctcttctcttttccttgctgtccctggggctggaggagcacgggcctccccgggagtgggcttcagcctccctagactcctgtctccttccaagggctaggcctgggggaccagaagcaagaggtgagtgacggccggcagggtgggaggaggagggcccatgcctccccaaggcaaggtggggcaggacccccagggaccagcacggtcccggttgggaggggctggggcccggcagagggtgtggcaggtgtggacgtggcaacaggcggctcccgggggtctcgggggatggcgggctctgggccaatggcctcagtgaggactgccagggtctgaaggttgggcacagagttcccggacgccaaacactaggtcagagaaatggcttttctgtgacccctgaccccacattctgatttaagggtggccaaagtaggcctggccctggctggcctggggattgagctgagagcccagaagaaggctgaggcctgaggcctgggggactctgggctcctccaggccgcggctggacatccccctgaggcctggcccaggctggcgagggccggaggctgtgtccggctcctcggcaggcctggtggccggagcgtttctctgggccgctgtgcggtgcctgtggtaatgggctgttggcgttttgcaatgggccgggggtggggaggcggcgcacacatgcttcctgtggtgactgggcgcttcctgttttctcaggcgccggccttgctgctgccgatgtggaaacaggggcagctgcagcccgggcggctccaggctgggcgctgtgaccctgcccaga

SEQ ID NO: 7 - DNA, 257 bp, Homo sapiens
cctccccagcttagcccccaaagtcatccctctcagtcccagcttcctctgggcagggggcctggaccccatggcttcccaggccagccctttgagcaccacctgccccagccggccagagacttcctgtatgtagcgcaagagatttatgcaaacgggttggggcggtgatgtcaccccaaggggactatctcccagcggcaggcccttcgataaaatcaggaacttgtgctggccctgcaatgtcaagggagggg

SEQ ID NO: 8 - protein, 122 aa, Homo sapiens
HPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRH

SEQ ID NO: 9 - DNA, 116 bp, Homo sapiens
actacaggtggcacacatccatagtaccagctactccagaggctgaggcgggaggatcgcttgagcccaggaggtccaggctacagtgggccaagatcacgccattgcactccagc

SEQ ID NO: 10 - DNA, 91 bp, Homo sapiens
aataattagctgggcgtggtggcgggcgcctgtaatcccagctactcaggaggctgaggcaggagaatcatttgaacccgggaggcaaagt

SEQ ID NO: 11 - RNA, 3 nt, Artificial Sequence
  Synthetic Construct
  misc_feature (1)..(1): n is a, c, g, or u
ngg

SEQ ID NO: 12 - RNA, 3 nt, Artificial Sequence
  Synthetic Construct
  misc_feature (1)..(1): n is a, c, g, or u
nag

SEQ ID NO: 13 - RNA, 4 nt, Artificial Sequence
  Synthetic Construct
  misc_feature (1)..(1): n is a, c, g, or u
ngcg

SEQ ID NO: 14 - RNA, 4 nt, Artificial Sequence
  Synthetic Construct
  misc_feature (1)..(1): n is a, c, g, or u
ngag

SEQ ID NO: 15 - RNA, 4 nt, Artificial Sequence
  Synthetic Construct
  misc_feature (1)..(1): n is a, c, g, or u
  misc_feature (4)..(4): n is a, c, g, or u
ngan

SEQ ID NO: 16 - RNA, 4 nt, Artificial Sequence
  Synthetic Construct
  misc_feature (1)..(1): n is a, c, g, or u
  misc_feature (3)..(3): n is a, c, g, or u
ngng

SEQ ID NO: 17 - RNA, 6 nt, Artificial Sequence
  Synthetic Construct
  misc_feature (1)..(2): n is a, c, g, or t
nngrrt

SEQ ID NO: 18 - RNA, 5 nt, Artificial Sequence
  Synthetic Construct
  misc_feature (1)..(2): n is a, c, g, or u
nngrr

SEQ ID NO: 19 - RNA, 6 nt, Artificial Sequence
  Synthetic Construct
  misc_feature (1)..(3): n is a, c, g, or t
nnnrrt

SEQ ID NO: 20 - RNA, 4 nt, Artificial Sequence
  Synthetic Construct
tttv

SEQ ID NO: 21 - RNA, 4 nt, Artificial Sequence
  Synthetic Construct
tycv

SEQ ID NO: 22 - RNA, 4 nt, Artificial Sequence
  Synthetic Construct
tatv

SEQ ID NO: 23 - RNA, 8 nt, Artificial Sequence
  Synthetic Construct
  misc_feature (1)..(4): n is a, c, g, or t
nnnngatt

SEQ ID NO: 24 - RNA, 7 nt, Artificial Sequence
  Synthetic Construct
  misc_feature (1)..(2): n is a, c, g, or u
nnagaaw

SEQ ID NO: 25 - RNA, 5 nt, Artificial Sequence
  Synthetic Construct
  misc_feature (1)..(1): n is a, c, g, or u
  misc_feature (4)..(4): n is a, c, g, or u
nggng

SEQ ID NO: 26 - RNA, 6 nt, Artificial Sequence
  Synthetic Construct
  misc_feature (1)..(1): n is a, c, g, or u
naaaac

SEQ ID NO: 27 - RNA, 4 nt, Artificial Sequence
  Synthetic Construct
  misc_feature (1)..(1): n is a, c, g, or u
naar

SEQ ID NO: 28 - DNA, 20 bp, Artificial Sequence
  Synthetic Construct
ggcttcagcctccctagact

SEQ ID NO: 29 - DNA, 20 bp, Artificial Sequence
  Synthetic Construct
ctggtcagcaggaaattggt

SEQ ID NO: 30 - DNA, 20 bp, Artificial Sequence
  Synthetic Construct
gaaggaacacaggcctctcc

SEQ ID NO: 31 - DNA, 20 bp, Artificial Sequence
  Synthetic Construct
gagaccatgccagtctggtt

SEQ ID NO: 32 - DNA, 20 bp, Artificial Sequence
  Synthetic Construct
tcaccacaggaagcatgtgt

SEQ ID NO: 33 - DNA, 19 bp, Artificial Sequence
  Synthetic Construct
ggcagagttctccctgtgc

SEQ ID NO: 34 - DNA, 18 bp, Artificial Sequence
  Synthetic Construct
gtggagtcgccgattttt

SEQ ID NO: 35 - DNA, 20 bp, Artificial Sequence
  Synthetic Construct
gtgggctagtctgtggaagg

SEQ ID NO: 36 - DNA, 20 bp, Artificial Sequence
  Synthetic Construct
ccacgagacactatccagca

SEQ ID NO: 37 - DNA, 20 bp, Artificial Sequence
  Synthetic Construct
ggtctttggtgggttgaact

SEQ ID NO: 38 - DNA, 20 bp, Artificial Sequence
  Synthetic Construct
ttcccacccagcattacagt

SEQ ID NO: 39 - DNA, 20 bp, Artificial Sequence
  Synthetic Construct
ctaacagggctctccctgag

SEQ ID NO: 40 - DNA, 20 bp, Artificial Sequence
  Synthetic Construct
cagccattgaactcacttcg

SEQ ID NO: 41 - DNA, 23 bp, Artificial Sequence
  Synthetic Construct
cggaaaatagccttcgccatcac

SEQ ID NO: 42 - DNA, 22 bp, Artificial Sequence
  Synthetic Construct
atcactcgctccacctcatcct

SEQ ID NO: 43 - DNA, 21 bp, Artificial Sequence
  Synthetic Construct
tgttacaggcgtgcaaaatgg

SEQ ID NO: 44 - DNA, 21 bp, Artificial Sequence
  Synthetic Construct
tgcgtttggcgttggtataga

SEQ ID NO: 45 - DNA, 22 bp, Artificial Sequence
  Synthetic Construct
gtctcctctgacttcaacagcg

SEQ ID NO: 46 - DNA, 22 bp, Artificial Sequence
  Synthetic Construct
accaccctgttgctgtagccaa

SEQ ID NO: 47 - DNA, 20 bp, Artificial Sequence
  Synthetic Construct
gtgtctgctccctagctcca

SEQ ID NO: 48 - DNA, 20 bp, Artificial Sequence
  Synthetic Construct
atggcgtgtggtcacccaga

SEQ ID NO: 49 - DNA, 21 bp, Artificial Sequence
  Synthetic Construct
gacagtgctacatgggtgtga

SEQ ID NO: 50 - DNA, 20 bp, Artificial Sequence
  Synthetic Construct
ctttggagagtcccaagtgc

SEQ ID NO: 51 - DNA, 20 bp, Artificial Sequence
  Synthetic Construct
gagccatagcggtgagtacg

SEQ ID NO: 52 - DNA, 20 bp, Artificial Sequence
  Synthetic Construct
ttctccctggagagacctca

SEQ ID NO: 53 - DNA, 20 bp, Artificial Sequence
  Synthetic Construct
ggtgtgcaccaccatacttg

SEQ ID NO: 54 - DNA, 20 bp, Artificial Sequence
  Synthetic Construct
gccatgaaatgctctgctct

SEQ ID NO: 55 - DNA, 20 bp, Artificial Sequence
  Synthetic Construct
cctagcccttggaaggagac

SEQ ID NO: 56 - DNA, 18 bp, Artificial Sequence
  Synthetic Construct
cagccctttgagcaccac

SEQ ID NO: 57 - DNA, 24 bp, Artificial Sequence
  Synthetic Construct
gaagggcctgccgctgggagatag

SEQ ID NO: 58 - DNA, 20 bp, Artificial Sequence
  Synthetic Construct
aaaggcaactttcggaacgg

SEQ ID NO: 59 - DNA, 20 bp, Artificial Sequence
  Synthetic Construct
ttcctcaatctcgctctcgc

SEQ ID NO: 60 - DNA, 20 bp, Artificial Sequence
  Synthetic Construct
ggagccccttgaatcttagg

SEQ ID NO: 61 - DNA, 20 bp, Artificial Sequence
  Synthetic Construct
aaagcaggacaggaaagcaa

SEQ ID NO: 62 - DNA, 20 bp, Artificial Sequence
  Synthetic Construct
caggtggcacacatccatag

SEQ ID NO: 63 - DNA, 20 bp, Artificial Sequence
  Synthetic Construct
catgcttggccagttctttt

SEQ ID NO: 64 - DNA, 20 bp, Artificial Sequence
  Synthetic Construct
tcaacagatggctgtcttgg

SEQ ID NO: 65 - DNA, 20 bp, Artificial Sequence
  Synthetic Construct
tcagaagcctcatcccctta

SEQ ID NO: 66 - DNA, 20 bp, Artificial Sequence
  Synthetic Construct
ctgtggtaatgggctgttgg

SEQ ID NO: 67 - DNA, 18 bp, Artificial Sequence
  Synthetic Construct
ctctgggcagggtcacag

SEQ ID NO: 68 - DNA, 20 bp, Artificial Sequence
  Synthetic Construct
ggctgactccagaaagtgga

SEQ ID NO: 69 - DNA, 20 bp, Artificial Sequence
  Synthetic Construct
gggagaacgtgtagctctgc

SEQ ID NO: 70 - DNA, 25 bp, Artificial Sequence
  Synthetic Construct
ggctaatcctctatgggagtctgtc

SEQ ID NO: 71 - DNA, 22 bp, Artificial Sequence
  Synthetic Construct
ccaggtgctcaaggtcaacatc

SEQ ID NO: 72 - DNA, 58 bp, Artificial Sequence
  Synthetic Construct
aatgatacggcgaccaccgagatctacactctttccctacacgacgctcttccgatct

SEQ ID NO: 73 - DNA, 25 bp, Artificial Sequence
  Synthetic Construct
aatgatacggcgaccaccgagatct

SEQ ID NO: 74 - DNA, 39 bp, Artificial Sequence
  Synthetic Construct
gaccacgcgtatcgatgtcgactttttttttttttttta

SEQ ID NO: 75 - DNA, 39 bp, Artificial Sequence
  Synthetic Construct
gaccacgcgtatcgatgtcgacttttttttttttttttc

SEQ ID NO: 76 - DNA, 39 bp, Artificial Sequence
  Synthetic Construct
gaccacgcgtatcgatgtcgacttttttttttttttttg

SEQ ID NO: 77 - DNA, 20 bp, Artificial Sequence
  Synthetic Construct
ctgtctccttccaagggcta

SEQ ID NO: 78 - DNA, 22 bp, Artificial Sequence
  Synthetic Construct
gaccacgcgtatcgatgtcgac

SEQ ID NO: 79 - DNA, 20 bp, Artificial Sequence
  Synthetic Construct
  modified_base (20)..(20): 3 Biotin-TriEthylene glycol
aaggagacaggagtctaggg

SEQ ID NO: 80 - DNA, 19 bp, Artificial Sequence
  Synthetic Construct
  modified_base (19)..(19): 3 Biotin-TriEthylene glycol
tctggtcagcaggaaattg

SEQ ID NO: 81 - DNA, 20 bp, Artificial Sequence
  Synthetic Construct
  modified_base (20)..(20): 3 Biotin-TriEthylene glycol
cagagcaaaagaggggcaga

SEQ ID NO: 82 - DNA, 20 bp, Artificial Sequence
  Synthetic Construct
  modified_base (20)..(20): 3 Biotin-TriEthylene glycol
agaggagggacaacgaggag

SEQ ID NO: 83 - DNA, 20 bp, Artificial Sequence
  Synthetic Construct
  modified_base (20)..(20): 3 Biotin-TriEthylene glycol
caggacaagaggtgaggagg

SEQ ID NO: 84 - DNA, 20 bp, Artificial Sequence
  Synthetic Construct
  modified_base (20)..(20): 3 Biotin-TriEthylene glycol
gatctcacatcaccaagaca

SEQ ID NO: 85 - DNA, 20 bp, Artificial Sequence
  Synthetic Construct
  modified_base (20)..(20): 3 Biotin-TriEthylene glycol
cggtttggtaatccataacc

SEQ ID NO: 86 - DNA, 20 bp, Artificial Sequence
  Synthetic Construct
  modified_base (20)..(20): 3 Biotin-TriEthylene glycol
agtacatcagaagcctcatc

SEQ ID NO: 87 - DNA, 19 bp, Artificial Sequence
  Synthetic Construct
  modified_base (19)..(19): 3 Biotin-TriEthylene glycol
agggtcaataacctctgga

SEQ ID NO: 88 - DNA, 20 bp, Artificial Sequence
  Synthetic Construct
  modified_base (20)..(20): 3 Biotin-TriEthylene glycol
gctccaggagaaggaagata

SEQ ID NO: 89 - DNA, 19 bp, Artificial Sequence
  Synthetic Construct
  modified_base (19)..(19): 3 Biotin-TriEthylene glycol
tgctggttgtaagcaagga

SEQ ID NO: 90 - DNA, 20 bp, Artificial Sequence
  Synthetic Construct
  modified_base (20)..(20): 3 Biotin-TriEthylene glycol
gcaaagcaggacaggaaagc

SEQ ID NO: 91 - DNA, 19 bp, Artificial Sequence
  Synthetic Construct
  modified_base (19)..(19): 3 Biotin-TriEthylene glycol
gaaagcatgtctggctgag

SEQ ID NO: 92 - DNA, 19 bp, Artificial Sequence
  Synthetic Construct
  modified_base (19)..(19): 3 Biotin-TriEthylene glycol
ggtacacttggtctcaaag

SEQ ID NO: 93 - DNA, 20 bp, Artificial Sequence
  Synthetic Construct
  modified_base (20)..(20): 3 Biotin-TriEthylene glycol
ccagtgaatccgtaatcatg

SEQ ID NO: 94 - DNA, 20 bp, Artificial Sequence
  Synthetic Construct
  modified_base (20)..(20): 3 Biotin-TriEthylene glycol
gtagccagctttcatcaaca

SEQ ID NO: 95 - DNA, 20 bp, Artificial Sequence
  Synthetic Construct
  modified_base (20)..(20): 3 Biotin-TriEthylene glycol
atcttccagataactgccgt

SEQ ID NO: 96 - DNA, 20 bp, Artificial Sequence
  Synthetic Construct
  modified_base (20)..(20): 3 Biotin-TriEthylene glycol
ataatttcaccgccgaaagg

SEQ ID NO: 97 - DNA, 20 bp, Artificial Sequence
  Synthetic Construct
  modified_base (20)..(20): 3 Biotin-TriEthylene glycol
ttcatcagcaggatatcctg

SEQ ID NO: 98 - DNA, 20 bp, Artificial Sequence
  Synthetic Construct
  modified_base (20)..(20): 3 Biotin-TriEthylene glycol
tgatcacactcgggtgatta

SEQ ID NO: 99 - DNA, 20 bp, Artificial Sequence
  Synthetic Construct
  modified_base (20)..(20): 3 Biotin-TriEthylene glycol
aaacggggatactgacgaaa

SEQ ID NO: 100 - DNA, 20 bp, Artificial Sequence
  Synthetic Construct
  modified_base (20)..(20): 3 Biotin-TriEthylene glycol
gttatcgctatgacggaaca

SEQ ID NO: 101 - DNA, 20 bp, Artificial Sequence
  Synthetic Construct
  modified_base (20)..(20): 3 Biotin-TriEthylene glycol
tgtgaaagaaagcctgactg

SEQ ID NO: 102 - DNA, 20 bp, Artificial Sequence
  Synthetic Construct
  modified_base (20)..(20): 3 Biotin-TriEthylene glycol
gtaatcgccatttgaccact

SEQ ID NO: 103 - DNA, 22 bp, Artificial Sequence
  Synthetic Construct
  modified_base (1)..(1): Biotin
agggtgtggcaggtgtggacgt

SEQ ID NO: 104 - DNA, 22 bp, Artificial Sequence
  Synthetic Construct
acgtccacacctgccacaccct

SEQ ID NO: 105 - DNA, 22 bp, Artificial Sequence
  Synthetic Construct
  modified_base (1)..(1): Biotin
aggctctcacagctctcaacgt

SEQ ID NO: 106 - DNA, 22 bp, Artificial Sequence
  Synthetic Construct
acgttgagagctgtgagagcct

SEQ ID NO: 107 - DNA, 20 bp, Artificial Sequence
  Synthetic Construct
  modified_base (1)..(1): Biotin
cagtggtgtggcagagctac

SEQ ID NO: 108 - DNA, 20 bp, Artificial Sequence
  Synthetic Construct
gtagctctgccacaccactg

SEQ ID NO: 109 - DNA, 20 bp, Artificial Sequence
  Synthetic Construct
  modified_base (1)..(1): Biotin
cagtgctctcacagagctac

SEQ ID NO: 110 - DNA, 20 bp, Artificial Sequence
  Synthetic Construct
gtagctctgtgagagcactg

SEQ ID NO: 111 - DNA, 25 bp, Artificial Sequence
  Synthetic Construct
caccgcaggtggtctcagaggtcgg

SEQ ID NO: 112 - DNA, 25 bp, Artificial Sequence
  Synthetic Construct
aaacccgacctctgagaccacctgc

SEQ ID NO: 113 - DNA, 25 bp, Artificial Sequence
  Synthetic Construct
caccgcacaagatcaggtaacaagt

SEQ ID NO: 114 - DNA, 25 bp, Artificial Sequence
  Synthetic Construct
aaacacttgttacctgatcttgtgc

SEQ ID NO: 115 - DNA, 25 bp, Artificial Sequence
  Synthetic Construct
aaaccccaccaatatcagtaatacc

SEQ ID NO: 116 - DNA, 25 bp, Artificial Sequence
  Synthetic Construct
aaaccccaccaatatcagtaatacc

SEQ ID NO: 117 - DNA, 20 bp, Artificial Sequence
  Synthetic Construct
gagctgagagcccagaagaa

SEQ ID NO: 118 - DNA, 19 bp, Artificial Sequence
  Synthetic Construct
ctcggccttctcgcaaaga

SEQ ID NO: 119 - DNA, 21 bp, Artificial Sequence
  Synthetic Construct
gacagtgctacatgggtgtga

SEQ ID NO: 120 - DNA, 20 bp, Artificial Sequence
  Synthetic Construct
agggacaacgaggaggtttt

SEQ ID NO: 121 - DNA, 25 bp, Artificial Sequence
  Synthetic Construct
caccgagaactcctagcgggacact

SEQ ID NO: 122 - DNA, 25 bp, Artificial Sequence
  Synthetic Construct
aaacagtgtcccgctaggagttctc

SEQ ID NO: 123 - DNA, 25 bp, Artificial Sequence
  Synthetic Construct
caccgatggctgaggttgatggttg

SEQ ID NO: 124 - DNA, 25 bp, Artificial Sequence
  Synthetic Construct
aaaccaaccatcaacctcagccatc

SEQ ID NO: 125 - DNA, 41 bp, Artificial Sequence
  Synthetic Construct
aatttaggtgacactatagaactacaggtggcacacatcca

SEQ ID NO: 126 - DNA, 22 bp, Artificial Sequence
  Synthetic Construct
gctggagtgcaatggcgtgatc

SEQ ID NO: 127 - DNA, 39 bp, Artificial Sequence
  Synthetic Construct
aatttaggtgacactatagatcttggcccactgtagcct

SEQ ID NO: 128 - DNA, 22 bp, Artificial Sequence
  Synthetic Construct
actacaggtggcacacatccat

SEQ ID NO: 129 - DNA, 42 bp, Artificial Sequence
  Synthetic Construct
aatttaggtgacactatagaatacaataattagctgggcgtg

SEQ ID NO: 130 - DNA, 25 bp, Artificial Sequence
  Synthetic Construct
gtttcgctcttgttgcccaggctgg

SEQ ID NO: 131 - DNA, 44 bp, Artificial Sequence
  Synthetic Construct
aatttaggtgacactatagaacaacctctacggaaaagagtatg

SEQ ID NO: 132 - DNA, 30 bp, Artificial Sequence
  Synthetic Construct
cctttcttcttttctctctttttctttttc